(19) United States
(12) Patent Application Publication
(10) Pub. No.: US 2023/0263742 A1
(43) Pub. Date: Aug. 24, 2023

(54) A LIPID

(71) Applicant: Stemirna Therapeutics Co., Ltd., Shanghai (CN)

(72) Inventors: Yunsong CAI, Shanghai (CN); Lei HUANG, Shanghai (CN); Na LIU, Shanghai (CN); Yu HANG, Shanghai (CN); Weiguo YAO, Shanghai (CN); Yujian ZHANG, Shanghai (CN); Hangwen LI, Shanghai (CN); Haifa SHEN, Shanghai (CN)

(21) Appl. No.: 18/041,061

(22) PCT Filed: May 5, 2022
(86) PCT No.: PCT/CN2022/090878

§ 371 Date: Feb. 8, 2023

(30) Foreign Application Priority Data
May 6, 2021 (CN) 202110489039.4
Jan. 19, 2022 (CN) 2021110059560.9

(51) Int. Cl. A61K 9/51; C07C 229/12; A61K 31/7105

(52) U.S. Cl. CPC A61K 9/5123; C07C 229/12; A61K 31/7105

(57) ABSTRACT

The invention provides a lipid of Formula (I) and nanoparticle compositions containing the same. Nanoparticle compositions including therapeutic and/or prophylactics such as RNA are useful in the delivery of therapeutic and/or prophylactics to mammalian cells or organs to, for example, regulate polypeptide, protein, or gene expression.

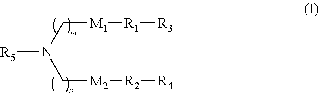

### RELATED APPLICATION

This application claims priorities to, and the benefits of, Chinese Application No. 202110489039.4, filed May 6, 2021; and Chinese Application No. 202210059560.9 filed Jan. 19, 2022; the entire contents of which are incorporated herein by reference in their entireties.

### TECHNICAL FIELD

The present invention relates to drug delivery systems, and particularly to a novel cationic and/or ionizable lipid, nanoparticle compositions comprising the same, and relevant product and method/application.

### BACKGROUND ART

Lipid-containing nanoparticle compositions, liposomes, and lipoplexes have proven effective as transport vehicles into cells and/or intracellular compartments for biologically active substances such as small molecule drugs, proteins, and nucleic acids. Such compositions generally include one or more “cationic” and/or amino (ionizable) lipids, phospholipids including polyunsaturated lipids, structural lipids (e.g., sterols), and/or lipids containing polyethylene glycol (PEG lipids). Cationic and/or ionizable lipids include, for example, amine-containing lipids that can be readily protonated.

Several amino lipid series have been developed for oligonucleotide delivery over the past couple of decades. (See Stanton M. G., Murphy-Benenato, K. E. RNA Therapeutics. Topics in Medicinal Chemistry, 2017, Vol 27., A. Garner eds., (Springer, Cham) p. 237-253, the content of which is incorporated by reference herein in its entirety). The first siRNA drug (Onpattro) approved in 2018 and two mRNA Coronavirus (COVID-19) vaccines approved in 2020 by FDA are all involved in the nanoparticle delivery system containing the cationic and/or ionizable lipids.

Although prominent impartments have been achieved, the cationic lipids containing nanoparticle delivery systems with increased efficiency are still desirable.

### SUMMARY

In an aspect, the present invention provides a lipid compound having the structure as shown in Formula (I):

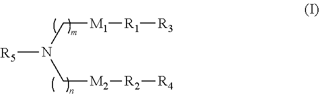

or a pharmaceutically acceptable salt thereof, wherein
R₁ and R₂ are each independently selected from the group consisting of C₁-C₁₂ alkyl and C₂-C₁₂ alkenyl;
R₃ and R₄ are each independently selected from the group consisting of C₁-C₁₂ alkyl, C₂-C₁₂ alkenyl, C₆-C₁₀ aryl and 5-10 membered heteroaryl;
provided that at least one of R₃ and R₄ is C₆-C₁₀ aryl or 5-10 membered heteroaryl, and optionally, R₃ and R₄ are each independently substituted by t R₆, where t is an integer selected from 1-5;
R₆ is each independently selected from the group consisting of C₁-C₁₂ alkyl and C₂-C₁₂ alkenyl;
M₁ and M₂ are each independently selected from the group consisting of —OC(O)—, —C(O)O—, —SC(S)—, and —C(S)S—;
R₅ is selected from the group consisting of —C₁₋₁₂ alkylene-Q, Q is selected from the group consisting of —OR₇ and —SR₇, R₇ is independently selected from the group consisting of H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamide, C₆-C₁₀ aryl and 5-10 membered heteroaryl;
m and n are each independently an integer selected from 1-12.

In another aspect, the invention provides a nanoparticle composition, comprising the lipid compound as described herein or a pharmaceutically acceptable salt thereof, and a therapeutic or prophylactic agent.

In yet another aspect, the invention provides a pharmaceutical composition comprising the lipid compound or a pharmaceutically acceptable salt or the nanoparticle composition as described herein and an optional pharmaceutically acceptable excipient.

In another aspect, the invention provides a method of delivering a therapeutic or prophylactic (e.g., an mRNA) agent to a cell of a subject. This method includes administering to a subject (e.g., a mammal, such as a human) a nanoparticle or pharmaceutical composition including a therapeutic or prophylactic agent, in which administering involves contacting the cell with the nanoparticle or pharmaceutical composition, whereby the therapeutic or prophylactic agent is delivered to the cell. The lipid compound or a pharmaceutically acceptable salt, nanoparticle or pharmaceutical composition as described herein can be useful in delivering a therapeutic or prophylactic agent (e.g., an mRNA) to a subject. The invention provides use of the lipid compound or a pharmaceutically acceptable salt as described herein for the manufacture of a nanoparticle or pharmaceutical composition for delivering a therapeutic or prophylactic agent (e.g., an mRNA) to a subject.

In another aspect, the invention provides a method of producing a polypeptide of interest in a cell of a subject (e.g., a mammalian cell). The method includes contacting the cell with a nanoparticle or pharmaceutical composition including a mRNA encoding the polypeptide of interest, whereby the mRNA is capable of being translated in the cell to produce the polypeptide. The lipid compound or a pharmaceutically acceptable salt, nanoparticle or pharmaceutical composition can be useful in producing a polypeptide of interest in a cell of a subject (e.g., a mammalian cell), wherein the nanoparticle or pharmaceutical composition including a mRNA encoding the polypeptide of interest. The invention also provides use of the lipid compound or a pharmaceutically acceptable salt as described herein for the manufacture of a nanoparticle or pharmaceutical composition for producing a polypeptide of interest in a cell of a subject (e.g., a mammalian cell), wherein the nanoparticle or pharmaceutical composition including a mRNA encoding the polypeptide of interest.

In another aspect, the invention provides a method of treating or preventing a disease or disorder in a subject (e.g., mammal, particularly a human) in need thereof. The method includes administering to the subject a therapeutically or prophylactically effective amount of a nanoparticle or pharmaceutical composition as described herein. The nanoparticle or pharmaceutical composition as described herein can be useful for treating or preventing a disease or disorder in a subject (e.g., mammal, particularly a human) in need thereof. The invention also provides use of the nanoparticle or pharmaceutical composition as described herein for the manufacture of a medicament for treating or preventing a disease or disorder in a subject (e.g., mammal, particularly a human) in need thereof.

In some embodiments, the disease or disorder is characterized by dysfunctional or aberrant protein or polypeptide activity. For example, the disease or disorder is selected from the group consisting of rare diseases, infectious diseases, cancer and proliferative diseases, genetic diseases (e.g., cystic fibrosis), autoimmune diseases, diabetes, neurodegenerative diseases, cardio- and reno-vascular diseases, and metabolic diseases.

In yet another aspect, the invention provides a method of delivering (e.g., specifically delivering) a therapeutic or prophylactic agent to an organ (e.g., a liver) of a subject. This method includes administering to a subject (e.g., a mammal, particularly a human) a nanoparticle or pharmaceutical composition as described herein, in which administering involves contacting the cell with the nanoparticle or pharmaceutical composition, whereby the therapeutic or prophylactic agent is delivered to the target organ (e.g., a liver). The lipid compound or a pharmaceutically acceptable salt, nanoparticle or pharmaceutical composition as described herein can be useful in delivering (e.g., specifically delivering) a therapeutic or prophylactic agent to an organ (e.g., a liver) of a subject. The invention also provides use of the lipid compound or a pharmaceutically acceptable salt as described herein for the manufacture of a nanoparticle or pharmaceutical composition for delivering a therapeutic or prophylactic agent to an organ (e.g., a liver) of a subject.

### DESCRIPTION OF THE DRAWINGS

FIG. 1 is a photograph of fluorescence observed under an in vivo imaging instrument upon intravenous administration of mRNA-LNP of MC3, SW-II-118, SW-II-120, and SW-II-121 of Example 3 to mice.

FIG. 2 is a schematic diagram of Luciferase expression in injection site and liver site upon intramuscular administration of mRNA-LNP of MC3, SW-II-118, SW-II-120, and SW-II-121 of Example 4 to mice overserved by an in vivo imager.

### DETAILED DESCRIPTION

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise. For example, the range “i-j” encompasses the range bounded by i and j and the two endpoints i and j, and also encompasses each point value therein and the subranges formed by those point values. For example, the range “1-12” may encompass, for example, 1-10, 1-8, 1-6, 1-5, 2-8, 5-7, and the like, as well as 1, 2, 3, 4, 5, 6, 7, 8, 9, 10, 11, 12, and the like.

Articles such as “a,” “an,” and “the” may mean one or more than one unless indicated to the contrary or otherwise evident from the context. Descriptions that include “or” between one or more members of a group are considered satisfied if one, more than one, or all of the group members are present in, employed in, or otherwise relevant to a given product or process unless indicated to the contrary or otherwise evident from the context. The present invention includes embodiments in which exactly one member of the group is present in, employed in, or otherwise relevant to a given product or process. The present invention includes embodiments in which more than one, or all, of the group members are present in, employed in, or otherwise relevant to a given product or process. As used herein, “one or more” and “at least one” refer to, for example, 1, 2, 3, 4, 5, 6, 7, 8, 9, 10, 11, 12, 13, 14, 15, 16, 17, 18, 19, 20 or more.

The term “comprising” is intended to be open and permits but does not require the inclusion of additional elements or steps. When the term “comprising” is used herein, the terms “consisting essentially of” and “consisting of” are thus also encompassed and disclosed. Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

As used herein, the term “alkyl” means a linear or branched, saturated hydrocarbon including one or more carbon atoms, which is optionally substituted. The term “C₁-C₁₂ alkyl” or “C₁₋₁₂ alkyl” means an optionally substituted linear or branched, saturated hydrocarbon including 1-12 carbon atoms. As used herein, the term “alkoxyl” means the alkyl as described herein, which is linked to the rest of the molecule via an oxygen atom. The “alkylene” refers to a divalent group formed from the corresponding alkyl group losing a hydrogen atom. The term “C₁-C₁₂ alkylene” or “C₁₋₁₂ alkylene” means an optionally substituted linear or branched, alkylene including 1-12 carbon atoms.

As used herein, the term “alkenyl” means a linear or branched hydrocarbon including two or more carbon atoms and at least one double bond, which is optionally substituted. The term “C₂-C₁₂ alkenyl” or “C₂₋₁₂ alkenyl” means an optionally substituted linear or branched hydrocarbon including 2-12 carbon atoms and at least one carbon-carbon double bond. An alkenyl group may include one, two, three, four, or more carbon-carbon double bonds.

As used herein, the term “aryl” refers to an all-carbon monocyclic or fused polycyclic aromatic ring group having a conjugated electron system. For example, a C₆-C₁₀ aryl group may have 6-10 carbon atoms, e.g., 6, 7, 8, 9, 10 carbon atoms. Examples of aryl groups include, but are not limited to, phenyl, naphthyl, and the like.

As used herein, the term “heteroaryl” refers to a monocyclic or fused polycyclic ring system containing at least one ring atom selected from N, O, S, the remaining ring atoms being C, and having at least one aromatic ring. A heteroaryl group may have 5-10 ring atoms (5-10 membered heteroaryl groups) including 5, 6, 7, 8, 9 or 10 membered, especially 5 or 6 membered heteroaryl groups. Examples of heteroaryl groups include, but are not limited to, pyrrolyl, furyl, thienyl, imidazolyl, oxazolyl, pyrazolyl, pyridyl, pyrimidinyl, pyrazinyl, quinolinyl, isoquinolinyl, tetrazolyl, triazolyl, triazinyl, benzofuranyl, benzothienyl, indolyl, isoindolyl and the like.

The groups/substituents as described herein (e.g., any one of R₁-R₇, such as alkyl, alkenyl, aryl, amino etc.) may be optionally substituted unless otherwise specified. Optional substituents may be selected from the group consisting of, but are not limited to, a halogen atom (e.g., a chloride, bromide fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g. —C(O)X, in which X is a halide selected from bromide, fluoride, chloride, and iodide), a carbonate (e.g., —OC(O)OR), an alkoxy (e.g., —OR), an acetal (e.g. —C(OR)₂R″″, in which each OR are alkoxy groups that can be the same or different and R″″ is an alkyl or alkenyl group), a phosphate (e.g., P(O)₄³⁻), a thiol (e.g., —SH), a sulfoxide (e.g., —S(O)R), a sulfinic acid (e.g., —S(O)OH), a sulfonic acid (e.g., —S(O)₂OH), a thial (e.g., —C(S)H), a sulfate (e.g., S(O)₄²⁻), a sulfonyl (e.g., —S(O)₂—), an amide (e.g., —C(O)NR₂ or —N(R)C(O)R), an azido (e.g., —N₃), a nitro (e.g., —NO₂), a cyano (e.g., —CN), an isocyano (e.g., —NC), an acyloxy (e.g., —OC(O)R), an amino (e.g., —NR₂, —NRH, or —NH₂), a carbamoyl (e.g., —OC(O)NR₂, —OC(O)NRH, —OC(O)NH₂), a sulfonamide, (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H). In any of the preceding, R is an alkyl, alkoxyl, aryl, heteroaryl or alkenyl group, as defined herein. In some embodiments, the substituent groups themselves may be further substituted with, for example, one, two, three, four, five, or six substituents as defined herein. For example, an alkyl group may be further substituted with one, two, three, four, five, or six substituents as described herein.

As used herein, the terms “approximately” and “about” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

As used herein, the term “compound” is meant to include all isotopes of the structure depicted. “Isotopes” refers to atoms having the same atomic number but different mass numbers resulting from a different number of neutrons in the nuclei, e.g., deuterium (D) isotope. For example, isotopes of hydrogen include tritium and deuterium. Further, a compound, salt, or complex of the present invention can be prepared in combination with solvent or water molecules to form solvates and hydrates by routine methods.

In certain aspects, the invention also includes methods of synthesizing a compound of any of Formulae (I), (II), (III), or (IV) and intermediate(s) for synthesizing the compound.

As used herein, the term “contacting” refers to establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” refers to providing an entity to a destination. For example, delivering a therapeutic or prophylactic agent to a subject may involve administering a nanoparticle composition including the therapeutic or prophylactic agent to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the ratio of the amount of a therapeutic or prophylactic agent that becomes part of a nanoparticle composition, relative to the initial total amount of therapeutic or prophylactic agent used in the preparation of a nanoparticle composition. For example, if 97 mg of therapeutic or prophylactic agent are encapsulated in a nanoparticle composition out of a total 100 mg of therapeutic or prophylactic agent initially provided to the nanoparticle composition, the encapsulation efficiency may be given as 97%. As used herein, “encapsulation” may refer to complete, substantial, or partial enclosure, confinement, surrounding, or encasement.

As used herein, “expression” of a nucleic acid sequence refers to translation of an mRNA into a polypeptide or protein and/or post-translational modification of a polypeptide or protein.

As used herein, a “lipid component” is that component of a nanoparticle composition that includes one or more lipids. For example, the lipid component may include one or more cationic/ionizable, PEGylated, structural, or other lipids, such as phospholipids.

As used herein, “modified” refers to non-natural. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

As used herein, “subject” refers to a target subject to whom some treatment is intended. Subject which is expected to be administered these compositions include, but are not limited to, a human, other primate, and other mammal, such as a cow, a pig, a horse, a sheep, a cat, a dog, a mouse, or a rat. Preferably, the subject may be a mammal, especially a human.

As used herein, “patient” refers to a subject who may seek or be in need of treatment, requires treatment, is receiving treatment, will receive treatment, or a subject who is under care by a trained professional for a particular disease or condition.

The phrase “pharmaceutically acceptable” is used herein to refer to those compounds, salts, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammomum, tetraethylammomum, methylamine, dimethylamine, trimethylamine, triethylamine, ethylamine, and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

As used herein, the “polydispersity index” or “PDI” is a ratio that describes the homogeneity of the particle size distribution of a system. A small value, e.g., less than 0.3, indicates a narrow particle size distribution.

As used herein, the term “polypeptide” or “polypeptide of interest” refers to a polymer of amino acid residues typically joined by peptide bonds that can be produced naturally (e.g., isolated or purified) or synthetically.

As used herein an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a poly A sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting small interfering RNA (siRNA), asymmetrical interfering RNA (aiRNA), microRNA (miRNA), Dicer-substrate RNA (dsRNA), small hairpin RNA (shRNA), mRNA, single-guide RNA (sgRNA), cas9 mRNA, and mixtures thereof.

As used herein, “size” or “mean size” in the context of nanoparticle compositions refers to the mean diameter of a nanoparticle composition.

As used herein, “targeted cells” refers to any one or more cells of interest. The cells may be found in vitro, in vivo, in situ, or in the tissue or organ of an organism. The organism may be an animal, preferably a mammal, more preferably a human and most preferably a patient.

As used herein “target tissue” refers to any one or more tissue types of interest in which the delivery of a therapeutic or prophylactic would result in a desired biological and/or pharmacological effect. Examples of target tissues include specific tissues, organs, and systems or groups thereof. In particular applications, a target tissue may be a kidney, a lung, a spleen, vascular endothelium in vessels (e.g., intra-coronary or intra-femoral), or tumor tissue (e.g., via intratumoral injection). An “off-target tissue” refers to any one or more tissue types in which the expression of the encoded protein does not result in a desired biological and/or pharmacological effect. In particular applications, off-target tissues may include the liver and the spleen.

As used herein, the term “therapeutically effective amount” refers to an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, or condition, to treat, improve symptoms of, diagnose, prevent, or delay the onset of the infection, disease, disorder, or condition.

As used herein, the term “treating” refers to partially or completely alleviating, ameliorating, improving, relieving, delaying onset of, inhibiting progression of, reducing severity of, or reducing incidence of one or more symptoms or features of a particular infection, disease, disorder, or condition. “Prevention” refers to preventing a potential disease or preventing the deterioration of symptoms or the development of a disease.

As used herein, the “ζ (zeta) potential” is e.g., the electrokinetic potential of a lipid in a particle composition.

### Lipid Compounds

In an aspect, the present invention provides a lipid compound having the structure as shown in Formula (I):

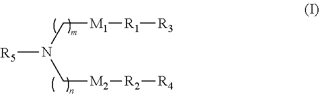

or a pharmaceutically acceptable salt thereof, wherein
R₁ and R₂ are each independently selected from the group consisting of C₁-C₁₂ alkyl and C₂-C₁₂ alkenyl;
R₃ and R₄ are each independently selected from the group consisting of C₁-C₁₂ alkyl, C₂-C₁₂ alkenyl, C₆-C₁₀ aryl and 5-10 membered heteroaryl;
provided that at least one of R₃ and R₄ is C₆-C₁₀ aryl or 5-10 membered heteroaryl, and optionally, R₃ and R₄ are each independently substituted by t R₆, where t is an integer selected from 1-5;
R₆ is each independently selected from the group consisting of C₁-C₁₂ alkyl and C₂-C₁₂ alkenyl;
M₁ and M₂ are each independently selected from the group consisting of —OC(O)—, —C(O)O—, —SC(S)—, and —C(S)S—;
R₅ is selected from the group consisting of —C₁₋₁₂ alkylene-Q, Q is selected from the group consisting of —OR₇ and —SR₇, R₇ is independently selected from the group consisting of H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamide, C₆-C₁₀ aryl and 5-10 membered heteroaryl;
m and n are each independently an integer selected from 1-12.

In an embodiment, R₁ is selected from the group consisting of C₁-C₁₂ alkyl. In another embodiment, R₁ is selected from the group consisting of C₁-C₆ alkyl.

In an embodiment, R₂ is selected from the group consisting of C₁-C₁₂ alkyl. In another embodiment, R₂ is selected from the group consisting of C₁-C₆ alkyl.

In an embodiment, one of R₃ and R₄ is C₆-C₁₀ aryl or 5-10 membered heteroaryl, and the other is C₁-C₁₂ alkyl or C₂-C₁₂ alkenyl.

In a specific embodiment, R₃ and R₄ are each independently selected from the group consisting of C₁-C₁₂ alkyl and phenyl, provided that at least one of R₃ and R₄ is phenyl. In another embodiment, one of R₃ and R₄ is phenyl, and the other is C₁-C₁₂ alkyl.

In yet another embodiment, R₃ and R₄ are each independently substituted by t R₆, where t is an integer selected from 1-5; for example, 1, 2, 3, 4, or 5. Preferably, t is an integer of 1-3, for example, 1, 2, or 3, particularly 1 or 2.

In an embodiment, R₆ is each independently selected from the group consisting of C₁-C₁₂ alkyl, for example, C₁-C₁₀ alkyl.

In an embodiment, t is 1, the benzene ring is substituted by R₆ at the meta- or para-position on the relative to R₁ or R₂.

In another embodiment, t is 2, the benzene ring is substituted by R₆ at the meta- and para-position on relative to R₁ or R₂.

In an embodiment, R₂ is substituted by R₄ at the 1 or end position. The 1-position refers to the position of the C atom in R₂ which is directly connected to M₂. The end position refers to the position of the C atom in R₂ which is farthest from M₂. In a specific embodiment, R₄ is selected from the group consisting of C₁-C₁₂ alkyl and R₃ is phenyl.

In an embodiment, R₁ is substituted by R₃ at the 1 or end position. The 1-position refers to the position of the C atom in R₁ which is directly connected to M₁. The end position refers to the position of the C atom in R₁ which is farthest from M₁. In a specific embodiment, R₃ is selected from the group consisting of C₁-C₁₂ alkyl and R₄ is phenyl.

In an embodiment, M₁ and M₂ are each independently selected from the group consisting of —OC(O)— and —C(O)O—.

In an embodiment, R₅ is selected from the group consisting of —C₁₋₅ alkylene-Q, for example, C₁, C₂, C₃, C₄, or C₅ alkylene-Q. In an exemplary embodiment, R₅ is selected from the group consisting of —C₁₋₃ alkylene-Q, for example, C₁, C₂, or C₃ alkylene-Q.

In another embodiment, Q is selected from the group consisting of —OH and —SH, particularly —OH.

In some embodiments, m and n are each independently an integer selected from 2-9, for example, 2, 3, 4, 5, 6, 7, 8, or 9. Preferably, m and n are each independently an integer selected from 2-7, for example, 2, 3, 4, 5, 6, or 7, more preferably, m and n are each independently an integer selected from 5-7, for example, 5, 6, or 7.

In some embodiments, the compound of Formula (I) comprises the compound of Formula (II):

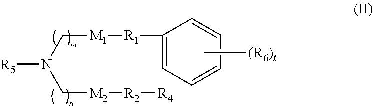

or a pharmaceutically acceptable salt thereof, wherein each of the groups is as defined herein.

In an embodiment,
R₁ is selected from the group consisting of C₁-C₆ alkyl;
R₂ is selected from the group consisting of C₁-C₁₀ alkyl;
R₄ is selected from the group consisting of C₁-C₁₀ alkyl;
M₁ and M₂ are each independently selected from the group consisting of —OC(O)— and —C(O)O—;
R₅ is selected from the group consisting of —C₁₋₅ alkylene-Q, Q is selected from the group consisting of —OR₇ and —SR₇, R₇ is independently selected from the group consisting of H, C₁-C₁₂ alkyl, C₂-C₁₂ alkenyl;
R₆ is each independently selected from the group consisting of C₁-C₁₂ alkyl and C₂-C₁₂ alkenyl, particularly C₁-C₁₂ alkyl;
m and n are each independently an integer selected from 2-9, for example, 2, 3, 4, 5, 6, 7, 8, or 9;
t is an integer selected from 1-3.

In an embodiment, R₅ is selected from the group consisting of —C₁₋₃ alkylene-Q, Q is selected from the group consisting of —OH and —SH, particularly —OH.

In an embodiment, m and n are each independently an integer selected from 2-7, for example, 2, 3, 4, 5, 6, or 7.

In some embodiments, t is 1 or 2.

In an embodiment, R₂ is substituted by R₄ at the 1 or end position. The 1-position refers to the position of the C atom in R₂ which is directly connected to M₂. The end position refers to the position of the C atom in R₂ which is farthest from M₂.

In an embodiment, t is 1, the benzene ring is substituted by R₆ at the meta- or para-position on relative to R₁.

In another embodiment, t is 2, the benzene ring is substituted by R₆ at the meta- and para-position on relative to R₁.

In some embodiments, the compound of Formula (I) comprises the compound of Formula (III):

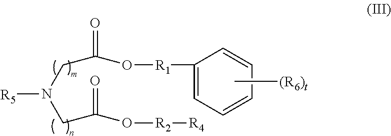

or a pharmaceutically acceptable salt thereof, wherein each of the groups is as defined herein.

In an embodiment,
R₁ is selected from the group consisting of C₁-C₆ alkyl;
R₂ is selected from the group consisting of C₁-C₁₀ alkyl;
R₄ is selected from the group consisting of C₁-C₁₀ alkyl;
R₅ is selected from the group consisting of —C₁₋₃ alkylene-Q, Q is selected from the group consisting of —OH and —SH, particularly —OH;
t is 1 or 2;
R₆ is selected from the group consisting of C₁-C₁₂ alkyl and C₂-C₁₂ alkenyl, particularly C₁-C₁₂ alkyl;
m and n are each independently an integer selected from 2-7, for example, 2, 3, 4, 5, 6, or 7.

In an embodiment, R₂ is substituted by R₄ at the 1 or end position. The 1-position refers to the position of the C atom in R₂ which is directly connected to

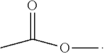

The end position refers to the position of the C atom in R₂ which is farthest from

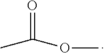

In an embodiment, t is 1, the benzene ring is substituted by R₆ at the meta- or para-position on relative to R₁.

In another embodiment, t is 2, the benzene ring is substituted by R₆ at the meta- and para-position on relative to R₁.

In some embodiments, the compound of Formula (I) comprises the compound of Formula (IV):

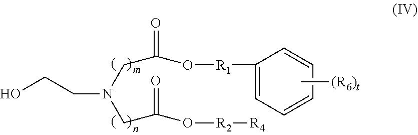

or a pharmaceutically acceptable salt thereof, wherein each of the groups is as defined herein.

In an embodiment,
R₁ is selected from the group consisting of C₁-C₆ alkyl;
R₂ is selected from the group consisting of C₁-C₁₀ alkyl;
R₄ is selected from the group consisting of C₁-C₁₀ alkyl;
t is 1 or 2;
R₆ is each independently selected from the group consisting of C₁-C₁₂ alkyl and C₂-C₁₂ alkenyl, particularly C₁-C₁₂ alkyl;
m and n are each independently an integer selected from 2-7, for example, 2, 3, 4, 5, 6, or 7.

In an embodiment, R₂ is substituted by R₄ at the 1 or end position. The 1-position refers to the position of the C atom in R₂ which is directly connected to

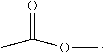

The end position refers to the position of the C atom in R₂ which is farthest from

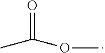

In an embodiment, t is 1, the benzene ring is substituted by R₆ at the meta- or para-position on relative to R₁.

In another embodiment, t is 2, the benzene ring is substituted by R₆ at the meta- and para-position on relative to R₁.

In a particular embodiment, the substituents (e.g., R₁-R₇) in the lipid compound of the present invention do not contain an alkenyl.

In a specific embodiment, the lipid compound of the present invention is selected from the group consisting of:

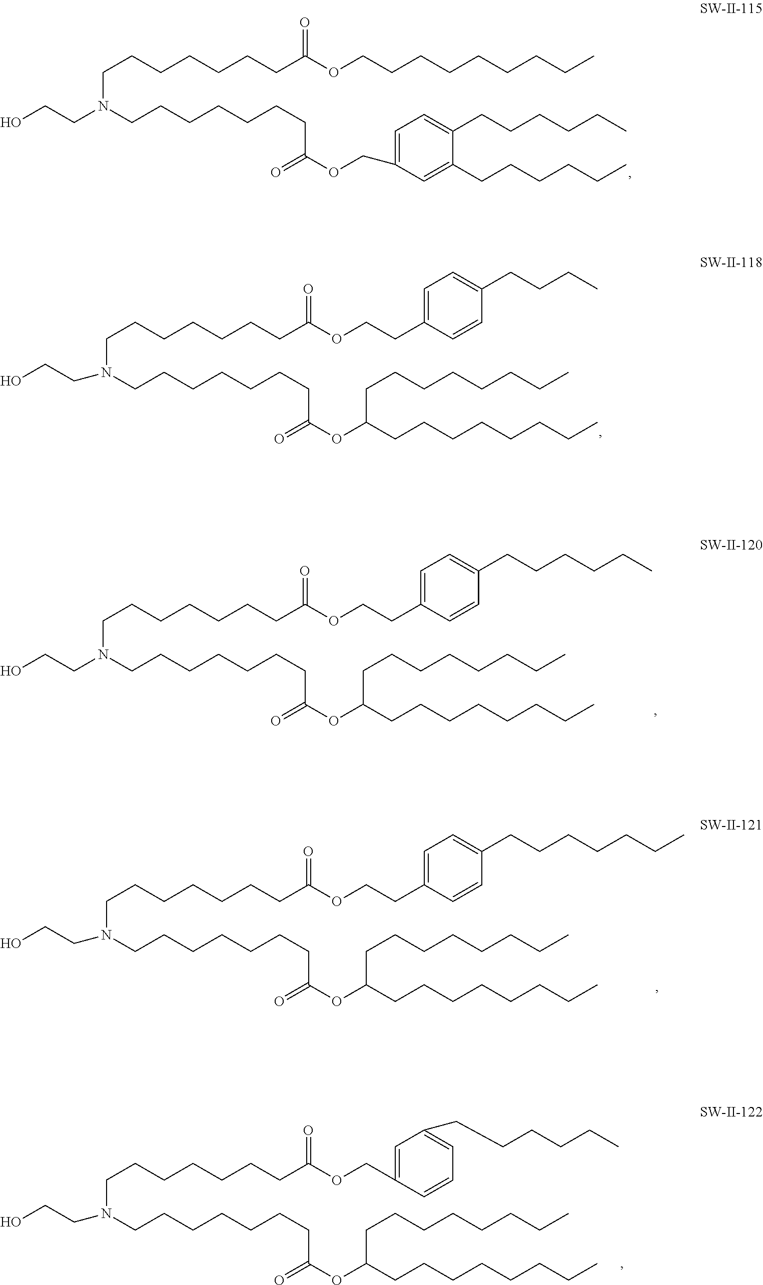 [SMILES: CCCCCCCCCOC(=O)CCCCCCCN(CCO)CCCCCCCC(=O)OCc1ccc(CCCCCC)c(CCCCCC)c1 ; CCCCCCCCC(CCCCCCCC)OC(=O)CCCCCCCN(CCO)CCCCCCCC(=O)OCCc1ccc(CCCC)cc1 ; CCCCCCCCC(CCCCCCCC)OC(=O)CCCCCCCN(CCO)CCCCCCCC(=O)OCCc1ccc(CCCCCC)cc1 ; CCCCCCCCC(CCCCCCCC)OC(=O)CCCCCCCN(CCO)CCCCCCCC(=O)OCCc1ccc(CCCCCCC)cc1 ; CCCCCCCCC(CCCCCCCC)OC(=O)CCCCCCCN(CCO)CCCCCCCC(=O)OCc1cccc(CCCCCC)c1]

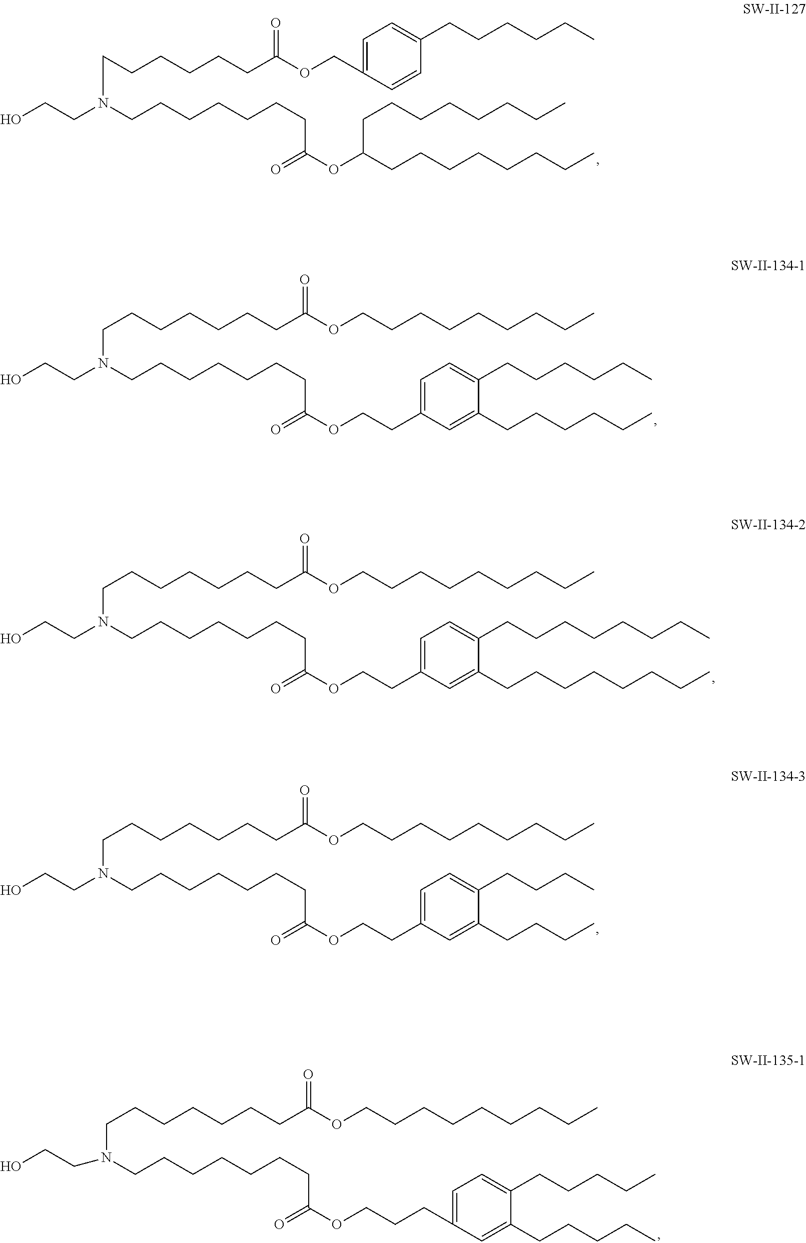 [SMILES: CCCCCCCCC(CCCCCCCC)OC(=O)CCCCCCCN(CCO)CCCCCCC(=O)OCc1ccc(CCCCCC)cc1 ; CCCCCCCCCOC(=O)CCCCCCCN(CCO)CCCCCCCC(=O)OCCc1ccc(CCCCCC)c(CCCCCC)c1 ; CCCCCCCCCOC(=O)CCCCCCCN(CCO)CCCCCCCC(=O)OCCc1ccc(CCCCCCCC)c(CCCCCCCC)c1 ; CCCCCCCCCOC(=O)CCCCCCCN(CCO)CCCCCCCC(=O)OCCc1ccc(CCCC)c(CCCC)c1 ; CCCCCCCCCOC(=O)CCCCCCCN(CCO)CCCCCCCC(=O)OCCCc1ccc(CCCCC)c(CCCCC)c1]

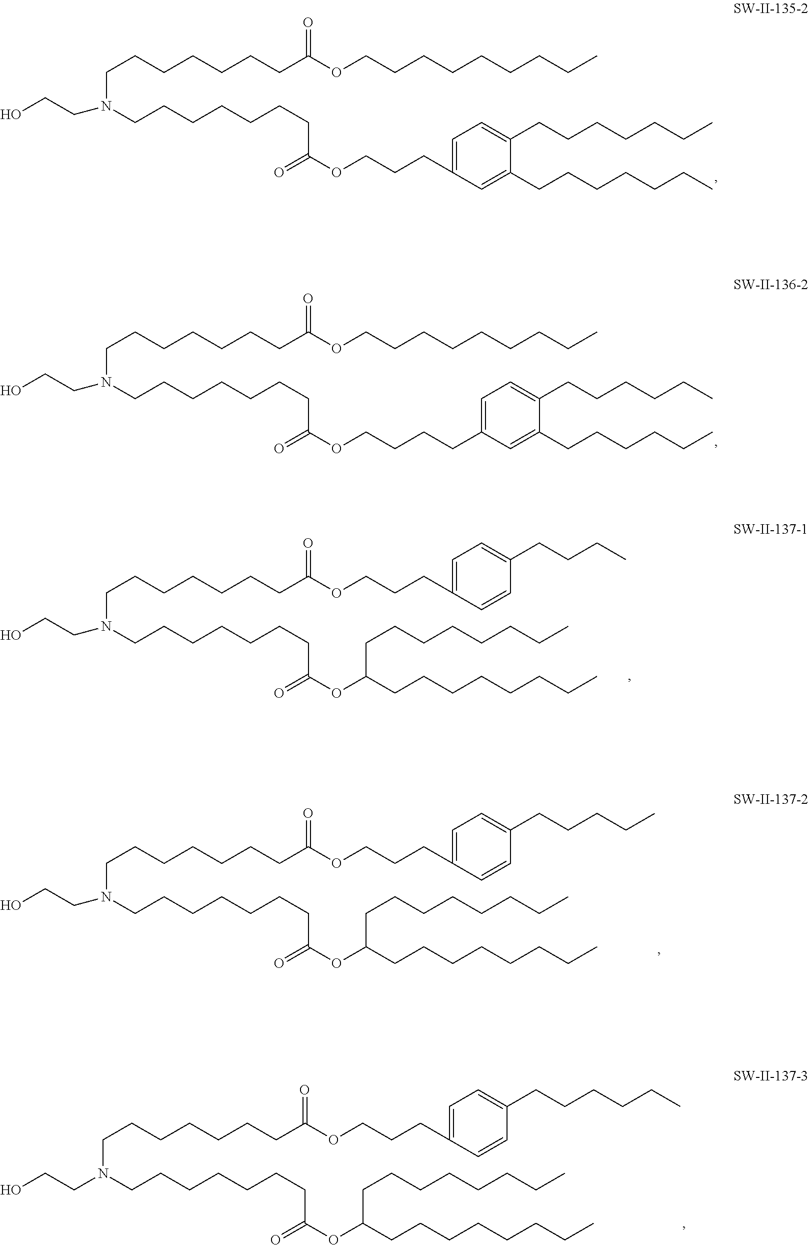 [SMILES: CCCCCCCCCOC(=O)CCCCCCCN(CCO)CCCCCCCC(=O)OCCCc1ccc(CCCCCCC)c(CCCCCCC)c1 ; CCCCCCCCCOC(=O)CCCCCCCN(CCO)CCCCCCCC(=O)OCCCCc1ccc(CCCCCC)c(CCCCCC)c1 ; CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCCC)cc1 ; CCCCCCCCC(CCCCCCCC)OC(=O)CCCCCCCN(CCO)CCCCCCCC(=O)OCCCc1ccc(CCCCCC)cc1]

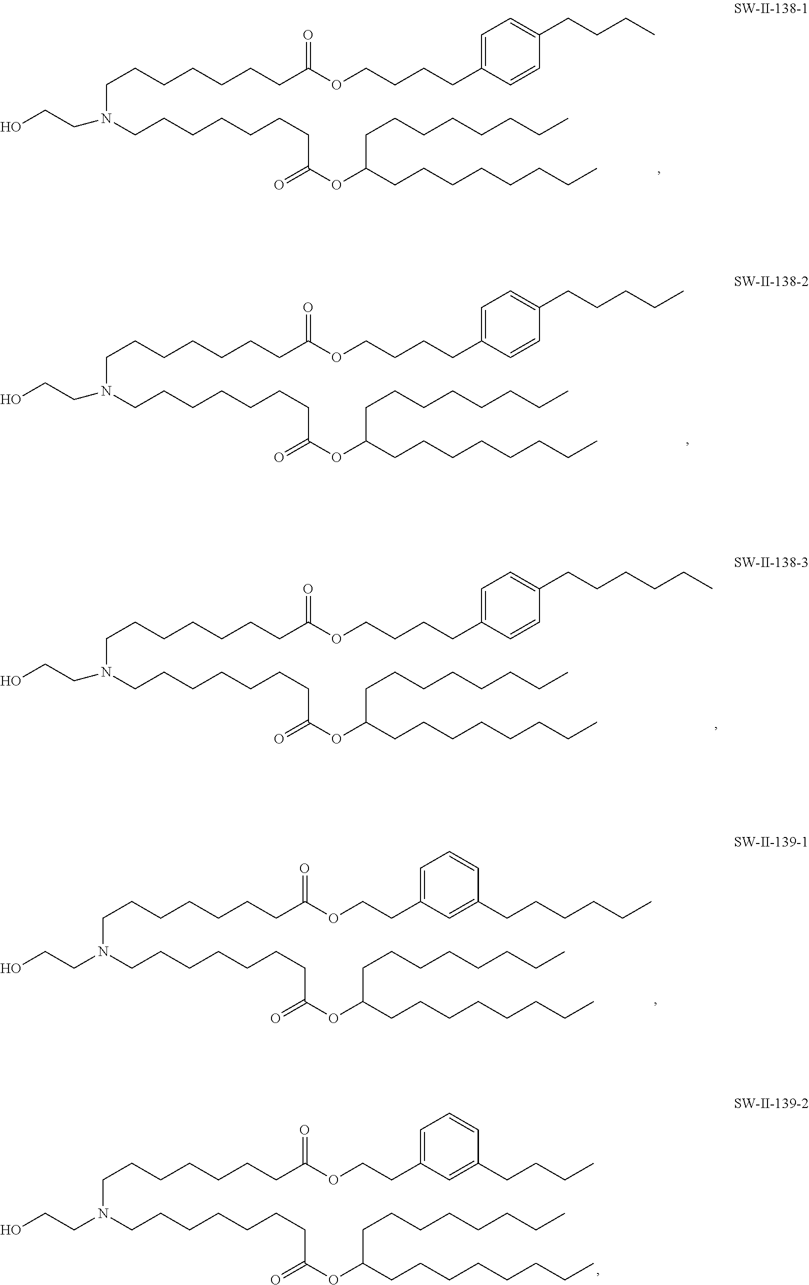 [SMILES: CCCCCCCCC(CCCCCCCC)OC(=O)CCCCCCCN(CCO)CCCCCCCC(=O)OCCCCc1ccc(CCCC)cc1 ; CCCCCCCCC(CCCCCCCC)OC(=O)CCCCCCCN(CCO)CCCCCCCC(=O)OCCCCc1ccc(CCCCC)cc1 ; CCCCCCCCC(CCCCCCCC)OC(=O)CCCCCCCN(CCO)CCCCCCCC(=O)OCCCCc1ccc(CCCCCC)cc1 ; CCCCCCCCC(CCCCCCCC)OC(=O)CCCCCCCN(CCO)CCCCCCCC(=O)OCCc1cccc(CCCCCC)c1 ; CCCCCCCCC(CCCCCCCC)OC(=O)CCCCCCCN(CCO)CCCCCCCC(=O)OCCc1cccc(CCCC)c1]

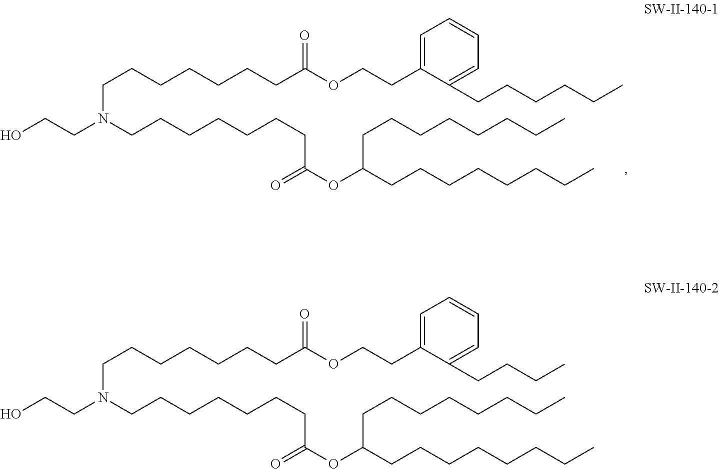 [SMILES: CCCCCCCCC(CCCCCCCC)OC(=O)CCCCCCCN(CCO)CCCCCCCC(=O)OCCc1ccccc1CCCCCC ; CCCCCCCCC(CCCCCCCC)OC(=O)CCCCCCCN(CCO)CCCCCCCC(=O)OCCc1ccccc1CCCC]

or a pharmaceutically acceptable salt thereof.

The lipid compound of the present invention, including those of formula (I), (II), (III) or (IV), may be cationic and/or ionizable, wherein the tertiary amine moiety may be protonated at physiological pH. Therefore, the lipid can be positively or partially positively charged at physiological pH. These lipids may be referred to as cationic or ionizable (amino) lipid. Lipid can also be zwitterionic. Such cationic, ionizable or zwitterionic forms, whether charged or not, are encompassed within the scope of the present invention.

### Nanoparticle Compositions

The invention also refers to nanoparticle compositions comprising a lipid component of the lipid compound of the present invention.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 80 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels.

Nanoparticle compositions may also include a variety of other components. For example, the lipid component of a nanoparticle composition may include one or more other lipids in addition to a lipid compound according to the present invention.

### Cationic/Ionizable Lipids

A nanoparticle composition may also include one or more cationic and/or ionizable lipids (e.g., lipids that may have a positive or partial positive charge at physiological pH) in addition to a lipid of the present invention (e.g., a lipid of Formula (I), (II), (III), or (IV)). Cationic and/or ionizable lipids may be selected from the nonlimiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, a cationic lipid may also be a lipid including a cyclic amine group.

### PEG Lipids

The lipid component of a nanoparticle composition may also include one or more PEG or PEG-modified lipids. Such species may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides (PEG-CER), PEG-modified dialkylamines, PEG-modified diacylglycerols (PEG-DEG), PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

### Structural Lipids

The lipid component of a nanoparticle composition may include one or more structural lipids. Structural lipids can be selected from the group consisting of, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

### Phospholipids

The lipid component of a nanoparticle composition may also include one or more phospholipids, such as one or more (poly)unsaturated lipids. Phospholipids may assemble into one or more lipid bilayers. In general, phospholipids may include a phospholipid moiety and one or more fatty acid moieties. For example, a phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component.

Phospholipids useful in the compositions and methods may be selected from the nonlimiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), and mixtures thereof. In some embodiments, a nanoparticle composition includes DSPC. In certain embodiments, a nanoparticle composition includes DOPE. In some embodiments, a nanoparticle composition includes both DSPC and DOPE.

In some embodiments, the lipid component of a nanoparticle composition includes a lipid of the present invention (e.g., a lipid of Formula (I), (II), (III), or (IV)), a phospholipid, a PEG lipid, and a structural lipid. In certain embodiments, the lipid component of the nanoparticle composition includes about 30 mol % to about 60 mol % compound of the present invention (e.g., compound of Formula (I), (II), (III), or (IV)), about 0 mol % to about mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % compound of the present invention (e.g., compound of Formula (I), (II), (III), or (IV)), about 5 mol % to about 25 mol % phospholipid, about 30 mol % to about mol % structural lipid, and about 0 mol % to about 10 mol % of PEG lipid. In a particular embodiment, the lipid component includes about 50 mol % said compound, about 10 mol % phospholipid, about 38.5 mol % structural lipid, and about 1.5 mol % of PEG lipid. In another particular embodiment, the lipid component includes about 40 mol % said compound, about mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In a specific embodiment, the nanoparticle composition comprises DSPC, DAPC (1,2-arachidonylphosphatidylcholine), DPPC, DOPE, DMPE, DSPE, DPPE or any combination thereof, particularly DSPC, DAPC, DPPC, DPPE or any combination thereof

### Adjuvants

In some embodiments, a nanoparticle composition that includes one or more lipids described herein may further include one or more adjuvants, e.g., Glucopyranosyl Lipid Adjuvant (GLA), CpG oligodeoxynucleotides (e.g., Class A or B), poly(I:C), aluminum hydroxide, and Pam3CSK4.

### Therapeutic Agents/Prophylactic Agents

Nanoparticle compositions may include one or more therapeutic or prophylactics. The invention provides methods of delivering a therapeutic or prophylactic to a mammalian cell or organ, producing a polypeptide of interest in a mammalian cell, and treating a disease or disorder in a mammal in need thereof comprising administering to a mammal or contacting a mammalian cell with a nanoparticle composition including a therapeutic or prophylactic.

Therapeutic or prophylactics include biologically active substances and are alternately referred to as “active agents”. A therapeutic or prophylactic may be a substance that, once delivered to a cell or organ, brings about a desirable change in the cell, organ, or other bodily tissue or system. Such species may be useful in the treatment of one or more diseases, disorders, or conditions. In some embodiments, a therapeutic or prophylactic is a small molecule drug useful in the treatment of a particular disease, disorder, or condition. Examples of drugs useful in the nanoparticle compositions include, but are not limited to, antineoplastic agents (e.g., vincristine, doxorubicin, mitoxantrone, camptothecin, cisplatin, bleomycm, cyclophosphamide, methotrexate, and streptozotocin), antitumor agents (e.g., actinomycin D, vincristine, vinblastine, cystine arabinoside, anthracyclines, alkylating agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetolol), antihypertensive agents (e.g., clonidine and hydralazine), anti-depressants (e.g., imipramine, amitriptyline, and doxepin), anti-conversants (e.g., phenytoin), antihistamines (e.g., diphenhydramine, chlorphenirimme, and promethazine), antibiotic/antibacterial agents (e.g., gentamycin, ciprofloxacin, and cefoxitin), antifungal agents (e.g., mconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some embodiments, a therapeutic or prophylactic is a cytotoxin, a radioactive ion, a chemotherapeutic, a vaccine, a compound that elicits an immune response, or another therapeutic or prophylactic. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol, rachelmycin (CC-1065), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium. Vaccines include compounds and preparations that are capable of providing immunity against one or more conditions related to infectious diseases such as influenza, measles, human papillomavirus (HPV), rabies, meningitis, whooping cough, tetanus, plague, hepatitis, and tuberculosis and can include mRNAs encoding infectious disease derived antigens and/or epitopes. Vaccines also include compounds and preparations that direct an immune response against cancer cells and can include mRNAs encoding tumor cell derived antigens, epitopes, and/or neoepitopes. Compounds eliciting immune responses may include vaccines, corticosteroids (e.g., dexamethasone), and other species. In some embodiments, a vaccine and/or a compound capable of eliciting an immune response is administered intramuscularly via a composition including a compound according to Formula (I), (II), (III), or (IV). Other therapeutic and/or prophylactics include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa, chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In other embodiments, a therapeutic or prophylactic is a protein. Therapeutic proteins useful in the nanoparticles in the invention include, but are not limited to, gentamycin, amikacin, insulin, erythropoietin (EPO), granulocyte-colony stimulating factor (G-CSF), granulocyte-macrophage colony stimulating factor (GM-CSF), Factor VIR, luteinizing hormone-releasing hormone (LHRH) analogs, interferons, heparin, Hepatitis B surface antigen, typhoid vaccine, and cholera vaccine.

### Polynucleotides and Nucleic Acids

In some embodiments, a therapeutic agent or prophylactic agent is a polynucleotide or nucleic acid (e.g., ribonucleic acid or deoxyribonucleic acid). The term “polynucleotide” in its broadest sense, includes any compound and/or substance that is or can be incorporated into an oligonucleotide chain. Exemplary polynucleotides for use in accordance with the present invention include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc.

In some embodiments, a therapeutic or prophylactic is an RNA. RNAs useful in the compositions and methods described herein can be selected from the group consisting of, but are not limited to, shortmers, antagomirs, antisense RNA, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and mixtures thereof.

In certain embodiments, a therapeutic or prophylactic is an mRNA. An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

In other embodiments, a therapeutic or prophylactic is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In certain embodiments, a therapeutic or prophylactic is an sgRNA and/or cas9 mRNA. sgRNA and/or cas9 mRNA can be used as gene editing tools. For example, an sgRNA-cas9 complex can affect mRNA translation of cellular genes.

In some embodiments, a therapeutic or prophylactic is an shRNA or a vector or plasmid encoding the same. An shRNA may be produced inside a target cell upon delivery of an appropriate construct to the nucleus. Constructs and mechanisms relating to shRNA are well known in the relevant arts.

### Other Components

A nanoparticle composition may include one or more components in addition to those described in the preceding sections. For example, a nanoparticle composition may include one or more small hydrophobic molecules such as a vitamin (e.g., vitamin A or vitamin E) or a sterol.

Nanoparticle compositions may also include one or more permeability enhancer molecules, carbohydrates, polymers, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a nanoparticle composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxy alkanoates, polypropylene fumarate, polyoxymethylene, poloxamers, polyoxamines, poly(ortho)esters, poly(butyric acid), poly(valeric acid), poly(lactide-co-caprolactone), trimethylene carbonate, poly(N-acryloylmorpholine) (PAcM), poly(-methyl-2-oxazoline) (PMOX), poly(-ethyl-2-oxazoline) (PEOZ), and polyglycerol.

Surface altering agents may include, but are not limited to, anionic proteins (e.g., bovine serum albumin), surfactants (e.g., cationic surfactants such as dimethyldioctadecylammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin β4, domase alfa, neltenexine, and erdosteine), and DNases (e.g., rhDNase). A surface altering agent may be disposed within a nanoparticle and/or on the surface of a nanoparticle composition (e.g., by coating, adsorption, covalent linkage, or other process).

In addition to these components, nanoparticle compositions may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art.

The amount of a therapeutic or prophylactic in a nanoparticle composition may depend on the size, composition, desired target and/or application, or other properties of the nanoparticle composition as well as on the properties of the therapeutic or prophylactic. For example, the amount of an RNA useful in a nanoparticle composition may depend on the size, sequence, and other characteristics of the RNA. The relative amounts of a therapeutic or prophylactic and other elements (e.g., lipids) in a nanoparticle composition may also vary. In some embodiments, the wt/wt ratio of the lipid component to a therapeutic or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic or prophylactic may be from about 10:1 to about 40:1. In certain embodiments, the wt/wt ratio is about 20:1. The amount of a therapeutic or prophylactic in a nanoparticle composition may, for example, be measured using absorption spectroscopy (e.g., ultraviolet-visible spectroscopy).

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta (ζ) potential.

The mean size of a nanoparticle composition may be between 10S of nm and 100S of nm, e.g., measured by dynamic light scattering (DLS). For example, the mean size may be from about 40 nm to about 250 nm, such as about 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm. In some embodiments, the mean size of a nanoparticle composition may be from about 50 nm to about 300 nm, from about 50 nm to about 290 nm, from about 50 nm to about 280 nm, from about 50 nm to about 270 nm, from about 50 nm to about 260 nm, from about 60 nm to about 300 nm, from about 60 nm to about 290 nm, from about 60 nm to about 280 nm, from about 60 nm to about 270 nm, from about 70 nm to about 300 nm, from about 70 nm to about 290 nm, from about 70 nm to about 280 nm, from about 70 nm to about 270 nm, from about 70 nm to about 260 nm, from about 80 nm to about 280 nm, from about 80 nm to about 270 nm, from about 80 nm to about 260 nm, from about 80 nm to about 250 nm, from about 90 nm to about 280 nm, from about 90 nm to about 270 nm, or from about 90 nm to about 260 nm. In certain embodiments, the mean size of a nanoparticle composition may be from about 90 nm to about 290 nm or from about 100 nm to about 250 nm. In a particular embodiment, the mean size may be about 100 nm. In other embodiments, the mean size may be about 150 nm. In other embodiments, the mean size may be about 200 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic or prophylactic describes the amount of therapeutic or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic or prophylactic in a solution containing the nanoparticle composition before and after breaking up the nanoparticle composition with one or more organic solvents or detergents. Fluorescence may be used to measure the amount of free therapeutic or prophylactic (e.g., RNA) in a solution. For the nanoparticle compositions described herein, the encapsulation efficiency of a therapeutic or prophylactic may be at least 50%, for example 50%, 55% 60%, 65%, 70% 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Alternatively, the nanoparticles can also be particles of a core-shell structure, in which the nucleic acid is comprised within a polyplex or protein core particles and further the polyplex or protein core particles are themselves encapsulated in a biocompatible lipid bilayer shell to form the present invention lipid nanoparticle. In some embodiments, the polyplex or protein core particles comprise positively-charged polymer or protein. In some embodiments, the positively-charged polymer or protein comprises protamine, polyethyleneimine, poly-(β-amino ester), or a combination thereof.

### Pharmaceutical Compositions

Nanoparticle compositions may be formulated in whole or in part as pharmaceutical compositions. Pharmaceutical compositions may include one or more nanoparticle compositions. For example, a pharmaceutical composition may include one or more nanoparticle compositions including one or more different therapeutic or prophylactics. Pharmaceutical compositions may further include one or more pharmaceutically acceptable excipients or accessory ingredients such as those described herein. Conventional excipients and accessory ingredients may be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use.

Relative amounts of the one or more nanoparticle compositions, the one or more pharmaceutically acceptable excipients, and/or any additional ingredients in a pharmaceutical composition in accordance with the present invention will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

mRNA Therapies

mRNA as a drug modality has the potential to deliver transmembrane and intracellular proteins, i.e., targets those standard biologics are unable to access due to the inability to cross the cell membrane. One major challenge to making mRNA based therapies a reality is the identification of an optimal delivery vehicle. Due to its large size, chemical instability and potential immunogenicity, mRNA requires a delivery vehicle that can offer protection from endo- and exo-nucleases, as well as shield the cargo from immune sentinels. Lipid nanoparticles (LNPs) have been identified as a leading option in this regard. This approach has recently been validated by demonstrating safe and effective delivery of an mRNA based vaccine formulated in LNPs.

Key performance criteria for a lipid nanoparticle delivery system are to maximize cellular uptake and enable efficient release of mRNA from the endosome. At the same time the LNP must provide a stable drug product and be able to be dosed safely at therapeutically relevant levels. LNPs are multi-component systems which typically consist of an amino lipid phospholipid, cholesterol, and a PEG-lipid. Each component is required for aspects of efficient delivery of the nucleic acid cargo and stability of the particle. The key component thought to drive cellular uptake, endosomal escape, and tolerability is the amino lipid. Cholesterol and the PEG-lipid contribute to the stability of the drug product both in vivo and on the shelf, while the phospholipid provides additional fusogenicity to the LNP, thus helping to drive endosomal escape and rendering the nucleic acid bioavailable in the cytosol of cells.

### Methods of Producing Polypeptides in Cells

The present invention provides methods of producing a polypeptide of interest in a mammalian cell. Methods of producing polypeptides involve contacting a cell with a nanoparticle composition including an mRNA encoding the polypeptide of interest. Upon contacting the cell with the nanoparticle composition, the mRNA may be taken up and translated in the cell to produce the polypeptide of interest.

In general, the step of contacting a mammalian cell with a nanoparticle composition including an mRNA encoding a polypeptide of interest may be performed in vivo, ex vivo, in culture, or in vitro. The amount of nanoparticle composition contacted with a cell, and/or the amount of mRNA therein, may depend on the type of cell or tissue being contacted, the means of administration, the physiochemical characteristics of the nanoparticle composition and the mRNA (e.g., size, charge, and chemical composition) therein, and other factors. In general, an effective amount of the nanoparticle composition will allow for efficient polypeptide production in the cell. Metrics for efficiency may include polypeptide translation (indicated by polypeptide expression), level of mRNA degradation, and immune response indicators.

The step of contacting a nanoparticle composition including an mRNA with a cell may involve or cause transfection. A phospholipid including in the lipid component of a nanoparticle composition may facilitate transfection and/or increase transfection efficiency, for example, by interacting and/or fusing with a cellular or intracellular membrane. Transfection may allow for the translation of the mRNA within the cell.

In some embodiments, the nanoparticle compositions described herein may be used therapeutically. For example, an mRNA included in a nanoparticle composition may encode a therapeutic polypeptide (e.g., in a translatable region) and produce the therapeutic polypeptide upon contacting and/or entry (e.g., transfection) into a cell. In other embodiments, an mRNA included in a nanoparticle composition may encode a polypeptide that may improve or increase the immunity of a subject. For example, an mRNA may encode a granulocyte-colony stimulating factor or trastuzumab.

In certain embodiments, an mRNA included in a nanoparticle composition may encode a recombinant polypeptide that may replace one or more polypeptides that may be substantially absent in a cell contacted with the nanoparticle composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

### Methods of Delivering Therapeutic Agents/Prophylactic Agents to Cells and Organs

The present invention provides methods of delivering a therapeutic or prophylactic to a mammalian cell or organ. Delivery of a therapeutic or prophylactic to a cell involves administering a nanoparticle composition including the therapeutic or prophylactic to a subject, where administration of the composition involves contacting the cell with the composition. For example, a protein, cytotoxic agent, radioactive ion, chemotherapeutic agent, or nucleic acid (such as an RNA, e.g., mRNA) may be delivered to a cell or organ. In the instance that a therapeutic or prophylactic is an mRNA, upon contacting a cell with the nanoparticle composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a nanoparticle composition may target a particular type or class of cells (e.g., cells of a particular organ or system thereof). For example, a nanoparticle composition including a therapeutic or prophylactic of interest may be specifically delivered to a mammalian liver, kidney, spleen, femur, or lung. Specific delivery to a particular class of cells, an organ, or a system or group thereof implies that a higher proportion of nanoparticle compositions including a therapeutic or prophylactic are delivered to the destination (e.g., tissue) of interest relative to other destinations, e.g., upon administration of a nanoparticle composition to a mammal. In some embodiments, specific delivery may result in a greater than 2 fold, 5 fold, 10 fold, 15 fold, or, 20 fold increase in the amount of therapeutic or prophylactic per 1 g of tissue of the targeted destination (e.g., tissue of interest, such as a liver) as compared to another destination (e.g., the spleen). In some embodiments, the tissue of interest is selected from the group consisting of a liver, kidney, a lung, a spleen, a femur, an ocular tissue (e.g., via intraocular, subretinal, or intravitreal injection), vascular endothelium in vessels (e.g., intra-coronary or intra-femoral) or kidney, and tumor tissue (e.g., via intratumoral injection).

As another example of targeted or specific delivery, an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other therapeutic or prophylactics or elements (e.g., one or more lipids or ligands) of a nanoparticle composition may be selected based on their affinity for particular receptors (e.g., low density lipoprotein receptors) such that a nanoparticle composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tribodies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell. For example, an estrogen receptor ligand, such as tamoxifen, can target cells to estrogen-dependent breast cancer cells that have an increased number of estrogen receptors on the cell surface. Other non-limiting examples of ligand/receptor interactions include CCR1 (e.g., for treatment of inflamed joint tissues or brain in rheumatoid arthritis, and/or multiple sclerosis), CCR7, CCR8 (e.g., targeting to lymph node tissue), CCR6, CCR9, CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), and VLA-4NCAM-1 (e.g., targeting to endothelium). In general, any receptor involved in targeting (e.g., cancer metastasis) can be harnessed for use in the methods and compositions described herein.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In some embodiments, a nanoparticle composition may target hepatocytes. Apolipoprotiens such as apolipoprotein E (apoE) have been shown to associate with neutral or near neutral lipid-containing nanoparticle compositions in the body, and are known to associate with receptors such as low-density lipoprotein receptors (LDLRs) found on the surface of hepatocytes. Thus, a nanoparticle composition including a lipid component with a neutral or near neutral charge that is administered to a subject may acquire apoE in a subject's body and may subsequently deliver a therapeutic or prophylactic (e.g., an RNA) to hepatocytes including LDLRs in a targeted manner.

### Methods of Treating Diseases and Disorders

Nanoparticle compositions may be useful for treating a disease, disorder, or condition. In particular, such compositions may be useful in treating a disease, disorder, or condition characterized by missing or aberrant protein or polypeptide activity. For example, a nanoparticle composition comprising an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Because translation may occur rapidly, the methods and compositions may be useful in the treatment of acute diseases, disorders, or conditions such as sepsis, stroke, and myocardial infarction. A therapeutic or prophylactic included in a nanoparticle composition may also be capable of altering the rate of transcription of a given species, thereby affecting gene expression.

Diseases, disorders, or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present invention provides a method for treating such diseases, disorders, or conditions in a subject by administering a nanoparticle composition including an RNA and a lipid component including a lipid according to Formula (I), (II), (III), or (IV), a phospholipid (optionally unsaturated), a PEG lipid, and a structural lipid, wherein the RNA may be an mRNA encoding a polypeptide that antagonizes or otherwise overcomes an aberrant protein activity present in the cell of the subject.

The invention provides methods involving administering nanoparticle compositions including one or more therapeutic or prophylactic agents and pharmaceutical compositions including the same. The terms therapeutic and prophylactic can be used interchangeably herein with respect to features and embodiments of the present invention. The compositions may be administered to a subject using any reasonable amount and any route of administration effective for preventing, treating, diagnosing diseases, disorders or conditions, or any other purpose. The specific amount administered to a given subject may vary depending on the species, age, and general condition of the subject; the purpose of the administration; the particular composition; the mode of administration; and the like. Compositions in accordance with the present invention may be formulated in dosage unit form for ease of administration and uniformity of dosage. It will be understood, however, that the total daily usage of a composition of the present invention will be decided by an attending physician within the scope of sound medical judgment. The specific therapeutically effective, prophylactically effective, or otherwise appropriate dose level for any particular patient will depend upon a variety of factors including the severity and identify of a disorder being treated, if any; the one or more therapeutic or prophylactics employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A nanoparticle composition may be administered by any route. In some embodiments, compositions are administered by one or more of a variety of routes, including oral, intravenous, intramuscular, intra-arterial, intramedullary, intrathecal, intraparenchymal, subcutaneous, intraventricular, trans- or intra-dermal, interdermal, rectal, intravaginal, intraperitoneal, intraocular, subretinal, intravitreal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. The present invention encompasses the delivery or administration of compositions described herein by any appropriate route taking into consideration likely advances in the sciences of drug delivery. In general, the most appropriate route of administration will depend upon a variety of factors including the nature of the nanoparticle composition including one or more therapeutic or prophylactics (e.g., its stability in various bodily environments such as the bloodstream and gastrointestinal tract), the condition of the patient (e.g., whether the patient is able to tolerate particular routes of administration), etc.

In certain embodiments, compositions in accordance with the present invention may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of a therapeutic or prophylactic (e.g., an mRNA) in a given dose, where a dose of 1 mg/kg (mpk) provides 1 mg of a therapeutic or prophylactic per 1 kg of subject body weight. In some embodiments, a dose of about 0.001 mg/kg to about 10 mg/kg of a therapeutic or prophylactic (e.g., mRNA) of a nanoparticle composition may be administered. In other embodiments, a dose of about 0.005 mg/kg to about 2.5 mg/kg of a therapeutic or prophylactic may be administered. In certain embodiments, a dose of about 0.1 mg/kg to about 1 mg/kg may be administered. In other embodiments, a dose of about 0.05 mg/kg to about 0.25 mg/kg may be administered. A dose may be administered one or more times per day, in the same or a different amount, to obtain a desired level of mRNA expression and/or therapeutic, diagnostic, prophylactic effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more therapeutic or prophylactics may be used in combination with one or more other therapeutic, prophylactic, diagnostic. It will further be appreciated that active agents utilized in combination may be administered together in a single composition or administered separately in different compositions. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects, such as infusion related reactions).

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Beneficial Effect

As described herein and shown in the Examples, the lipid compound of the present invention, and compositions comprising the same, can exhibit excellent delivery efficiency and expression efficiency. Moreover, the lipid compound of the present invention, and the composition containing the same, have no obvious cytotoxicity, and exhibit excellent safety. Furthermore, the effect is significantly better than those of the prior art, showing broad and excellent application prospects.

### EXAMPLES

### Example 1: Synthesis of Compounds

### General Considerations

All solvents and reagents used were obtained commercially and used as such unless noted otherwise. ¹H NMR spectra were recorded in CDCl₃, at 300 K using a Bruker Ultrashield 300 MHz instrument. Chemical shifts are reported as parts per million (ppm) relative to TMS (0.00) for 1H. Silica gel chromatographies were performed on ISCO CombiFlash Rf+ Lumen Instruments using ISCO RediSep Rf Gold Flash Cartridges (particle size: 20-40 microns). The procedures described below are useful in the synthesis of Compounds SW-II-115 to SW-II-140-2.

The following abbreviations are employed herein:
THF: Tetrahydrofuran
MeCN: Acetonitrile
LAH: Lithium Aluminum Hydride
DCM: Dichloromethane
DMAP: 4-Dimethylaminopyridine
LDA: Lithium Diisopropylamide
rt: Room Temperature
DME: 1,2-Dimethoxy ethane
n-BuLi: n-Butyllithium
CPME: Cyclopentyl methyl ether
EDCI: N-(3-dimethylaminopropyl)-N′-ethylcarbodiimide hydrochloride
DIEA: N,N-Diisopropylethylamine
PE: Petroleum ether
EA: Ethyl acetate

### A. Compound SW-II-115

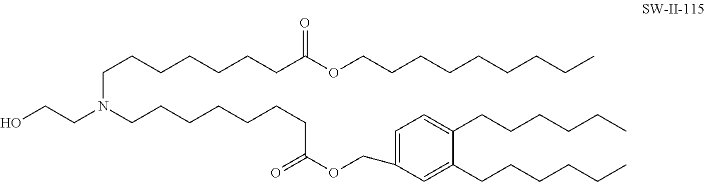 [SMILES: CCCCCCCCCOC(=O)CCCCCCCN(CCO)CCCCCCCC(=O)OCc1ccc(CCCCCC)c(CCCCCC)c1]

### 1. Synthesis of Intermediate 3

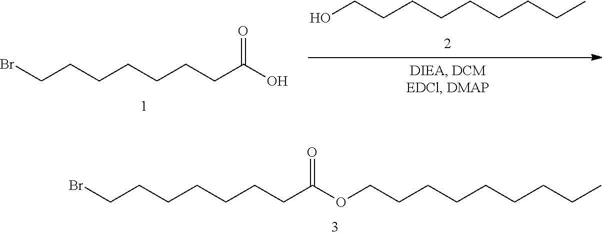

To a solution of compound 1 (10 g, 45 mmol, 1 eq.) and compound 2 (7.8 g, 54 mmol, 1.2 eq.) in DCM (100 mL) was added EDCI (17.3 g, 90 mmol, 2 eq.) and DMAP (2.2 g, 18 mmol, 0.4 eq.). Then DIEA (23.2 g, 180 mmol, 4 eq.) was added. The reaction mixture was stirred at room temperature under N₂ for 16 hours. TLC (petroleum ether:ethyl acetate=30:1) showed that compound 1 was consumed and the desired product was formed. The reaction mixture was diluted with DCM (20 mL) and washed with H₂O (40 mL), dried over anhydrous Na₂SO₄, filtered and concentrated under reduced pressure. The residue was purified by silica gel chromatography, eluting with petroleum ether:ethyl acetate (1:0-20:1), to obtain compound 3 (4.365 g, 28%) as a colorless oil.

### 2. Synthesis of Intermediate 5

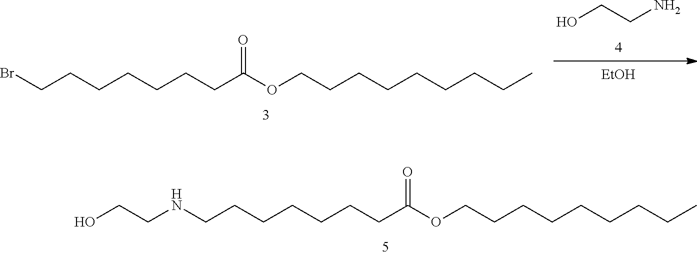

A solution of compound 3 (500 mg, 1.437 mmol, 1 eq.) and compound 4 (2.63 g, 43.103 mmol, 30 eq.) in EtOH was stirred at 60° C. for 16 hours under N₂. TLC (DCM:MeOH=10:1) showed that compound 3 was consumed, and TLC (DCM/MeOH=10/1) showed that new major spots were observed. The reaction mixture was concentrated under reduced pressure. The residue was diluted with EtOAc (50 mL) and washed with H₂O (3×50 mL). The organic layer was dried over anhydrous Na₂SO₄, filtered, and concentrated under reduced pressure. The residue was purified by silica gel chromatography, eluting with DCM/MeOH (1:0-10:1, v/v), to give compound 5 (264 mg, 56%) as a yellow oil.

### 3. Synthesis of Intermediate 8

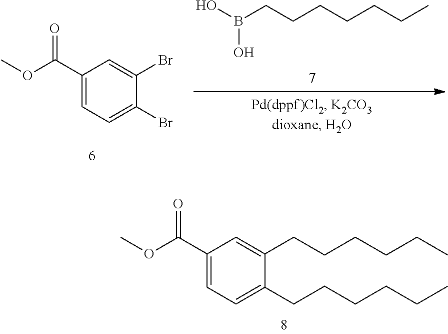

To a solution of compound 6 (500 mg, 1.712 mmol, 1 eq.) and compound 7 (1.113 g, 8.562 mmol, 5 eq) in a mixed solvent of dioxane/water (5 mL/0.5 mL) was added Pd(dppf)Cl₂ (112 mg, 0.171 mmol, 0.1 eq.) and potassium carbonate (709 mg, 5.136 mmol, 3 eq.). The mixture was stirred overnight at 100° C. under N₂. TLC (PE:EA=15:1) showed that the reaction was completed and new major spots were observed. The mixture was extracted with EA and washed with water. The organic layer was dried over anhydrous Na₂SO₄, filtered and concentrated in vacuo. The residue was purified by silica gel chromatography, eluting with PE:EA (1:0-10:1), to give compound 8 (455 mg, 88%) as a colorless oil.

### 4. Synthesis of Intermediate 9

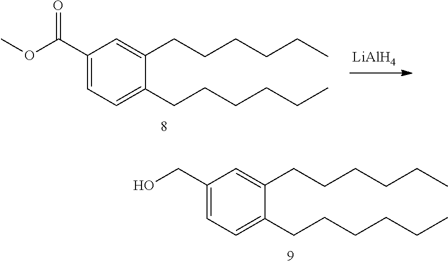

At 0° C. and under N₂, LiAlH₄ (1.5 mL, 1.497 mmol, 1M, in THF, 1 eq.) was added to a solution of compound 8 (455 mg, 1.497 mmol, 1 eq.) in THF (5 mL). The mixture was stirred at room temperature under N₂ for 2 hours. TLC (PE:EtOAc=5:1) showed that the reaction was completed and new major spots were observed. The mixture was quenched with water (1.5 mL) and treated with 2N HCl to adjust the pH between 6 and 7, extracted with EA and washed with brine. The organic layer was dried over anhydrous Na₂SO₄, filtered and concentrated in vacuo to obtain crude compound 9 (419 mg, >100%) as a colorless oil, which was used without further purification.

### 5. Synthesis of Intermediate 10

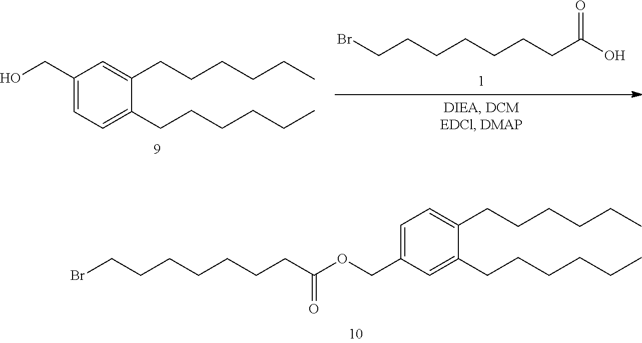 [SMILES: O=C(O)CCCCCCCBr ; CCCCCCc1ccc(CO)cc1CCCCCC ; CCCCCCc1ccc(COC(=O)CCCCCCCBr)cc1CCCCCC]

To a solution compound 1 (339 mg, 1.518 mmol, 1 eq.) and compound 9 (419 mg, 1.518 mmol, 1 eq.) in DCM (4 mL) was added EDCI (583 mg, 3.036 mmol, 2 eq.) and DMAP (74 mg, 0.607 mmol, 0.4 eq.). Then DIEA (783 mg, 6.072 mmol, 4 eq.) was added. The reaction mixture was stirred at room temperature under N₂ for 16 hours. TLC (petroleum ether: ethyl acetate=10:1) showed that the desired product was formed. The reaction mixture was extracted with EA and washed with water. The organic layer was dried over anhydrous Na₂SO₄, filtered, and concentrated in vacuo. The residue was purified by silica gel chromatography, eluting with petroleum ether:ethyl acetate (1:0-10:1), to obtain compound (443 mg, 60.7%) as a colorless oil.

### 6. Synthesis of the Final Product SW-II-115

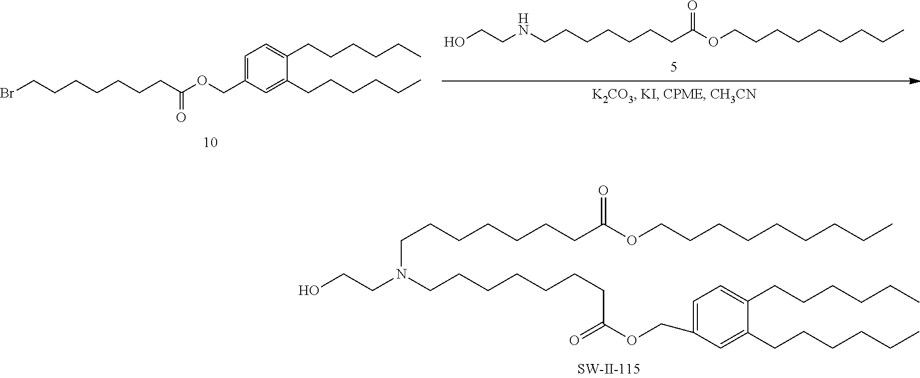 [SMILES: CCCCCCCCCOC(=O)CCCCCCCNCCO ; CCCCCCc1ccc(COC(=O)CCCCCCCBr)cc1CCCCCC ; CCCCCCCCCOC(=O)CCCCCCCN(CCO)CCCCCCCC(=O)OCc1ccc(CCCCCC)c(CCCCCC)c1]

To the mixed solvent CPME/CH₃CN (3 mL/3 mL) containing compound 10 (307 mg, 0.64 mmol, 1 eq.) and compound 5 (210 mg, 0.64 mmol, 1 eq.) was added K₂CO₃ (530 mg, 3.84 mmol, 6 eq.) and KI (212 mg, 1.28 mmol, 2 eq.). After the addition was completed, the mixture was stirred overnight at 90° C. under N₂. TLC (DCM:MeOH=10:1) showed that the reaction was completed and new major spots were observed. The mixture was extracted with EA and washed with water. The organic layer was dried over anhydrous Na₂SO₄, filtered, and concentrated under reduced pressure. The residue was purified by silica gel chromatography, eluting with DCM: MeOH (1:0-10:1, v/v), to give compound SW-II-115 (266 mg, 57%) as a yellow oil.

LCMS: Rt: 1.293 min; MS m/z (ELSD): 730.5 [M+H]⁺;

HPLC: 99.472% purity at ELSD; RT=4.895 min.

¹H NMR (400 MHz, CDCl₃) δ 7.21-6.99 (m, 3H), 5.05 (s, 2H), 4.05 (t, J=6.8 Hz, 2H), 3.58 (t, J=5.3 Hz, 2H), 2.69-2.46 (m, 10H), 2.31 (dt, J=20.0, 7.5 Hz, 4H), 1.69-1.18 (m, 51H), 0.89 (dt, J=12.4, 6.3 Hz, 9H).

¹³C NMR (101 MHz, CDCl₃) δ 173.90 (s), 173.68 (s), 140.80 (d, J=13.0 Hz), 133.31 (s), 129.25 (d, J=16.2 Hz), 128.30 (s), 125.75 (s), 77.30 (d, J=11.5 Hz), 77.04 (s), 76.72 (s), 66.22 (s), 64.43 (s), 58.12 (s), 55.72 (s), 53.90 (s), 34.32 (d, J=1.9 Hz), 32.69 (s), 32.48 (s), 31.81 (d, J=11.2 Hz), 31.25 (s), 29.59-28.91 (m), 28.66 (s), 27.17 (s), 26.64 (s), 25.94 (s), 24.91 (d, J=5.1 Hz), 22.65 (d, J=3.3 Hz), 14.10 (s).

### B. Compound SW-II-118

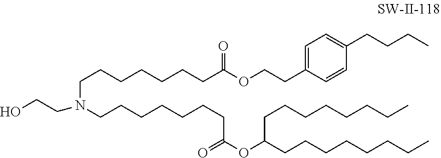 [SMILES: CCCCCCCCC(CCCCCCCC)OC(=O)CCCCCCCN(CCO)CCCCCCCC(=O)OCCc1ccc(CCCC)cc1]

### 1. Synthesis of Intermediate 3

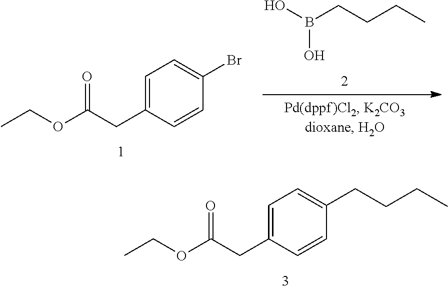

A solution of Compound 1 (1.22 g, 5.0 mmol, 1.0 eq.), Compound 2 (765 mg, 7.5 mmol, 1.5 eq.), Pd(PPh₃)₄ (tetrakistriphenylphosphine palladium, 289 mg, 0.25 mmol, 0.05 eq.) and K₂CO₃ (1.38 g, 10.0 mmol, 2.0 eq.) in toluene (10 ml) and H₂O (1 ml) was stirred at 110° C. under N₂ for 1 hour. TLC (petroleum ether:ethyl acetate=19:1) showed that compound 1 was consumed and a new spot was observed. The reaction mixture was diluted with DCM (50 mL) and washed with H₂O (40 mL), dried over anhydrous Na₂SO₄, filtered and concentrated under reduced pressure. The residue was purified by silica gel chromatography, eluting with petroleum ether:ethyl acetate (1:0-10:1), to obtain compound 3 (0.5 g, 45%) as a colorless oil. ¹H NMR (400 MHz, CDCl₃) δ 7.16 (dd, J=23.5, 8.1 Hz, 4H), 4.14 (q, J=7.1 Hz, 2H), 3.57 (s, 2H), 2.64-2.48 (m, 2H), 1.66-1.51 (m, 2H), 1.35 (dd, J=15.0, 7.4 Hz, 2H), 1.25 (t, J=7.1 Hz, 3H), 0.92 (t, J=7.3 Hz, 3H).

### 2. Synthesis of Intermediate 4

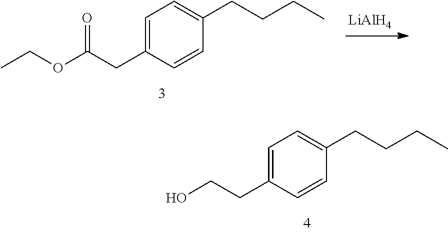

LiAlH₄ (193 mg, 5.09 mmol, 4.0 eq.) was added to a solution of compound 3 (280 mg, 1.27 mmol, 1.0 eq.) in THF (10 mL) at −78° C., and then the reaction was carried out at 10° C. for 3 hours. TLC showed that the reaction went well. The reaction was concentrated, diluted with Na₂SO₄ (20 mL) and extracted with EA (30 mL×2). The organic phase was dried over anhydrous Na₂SO₄, filtered and concentrated under reduced pressure to obtain compound 4 (3.12 g, crude) as a yellow oil.

### 3. Synthesis of Intermediate 6

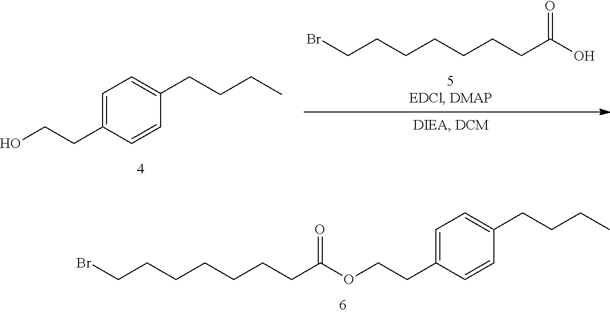

Solution of compound 4 (215 mg, 1.2 mmol, 1.0 eq.), compound 5 (404 mg, 1.8 mmol, 1.5 eq.), EDCI (1.15 g, 6.0 mmol, 5.0 eq.), DMAP (732 mg, 1.8 eq.), DIEA (1.29 g, 12.0 mmol, 10.0 eq.) and DIEA (1.29 g, 12.0 mmol, 10.0 eq.) in DCM (5 mL) were stirred at 10° C. for 16 h under N₂ protection. TLC (DCM:MeOH=10:1) showed that the reaction was completed and new major spots were observed. The mixture was concentrated under reduced pressure, and the residue was purified by silica gel chromatography, eluting with PE:EA (1:0-10:1, v/v), to obtain compound 6 (145 mg, 31%) as a colorless oil.

¹H NMR (400 MHz, CDCl₃) δ 7.12 (s, 4H), 4.27 (t, J=7.1 Hz, 2H), 3.52 (t, J=6.7 Hz, 1H), 3.40 (t, J=6.8 Hz, 1H), 2.90 (t, J=7.1 Hz, 2H), 2.65-2.50 (m, 2H), 2.28 (t, J=7.5 Hz, 2H), 1.93-1.70 (m, 2H), 1.64-1.56 (m, 4H), 1.44-1.27 (m, 8H), 0.92 (t, J=7.3 Hz, 3H).

### 4. Synthesis of the Final Product SW-II-118

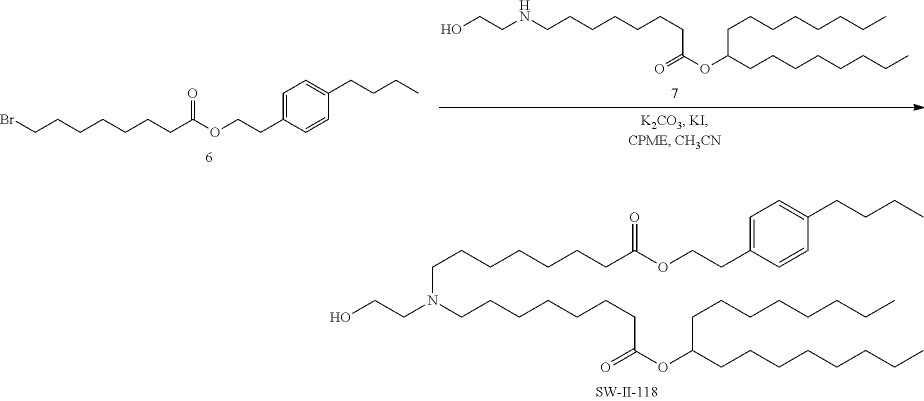

The mixture of compound 6 (140 mg, 0.37 mmol, 1.0 eq.), compound 7 (243 mg, 0.55 mmol, 1.5 eq.), K₂CO₃ (153 mg, 1.11 mmol, 3.0 eq.) and KI (123 mg, 0.74 mmol, 2.0 eq.) in a mixed solvent of CPME (1 mL) and CH₃CN (1 mL) was stirred under N₂ at 90° C. for 16 hours. The reaction mixture was concentrated under reduced pressure, and the residue was diluted with EtOAc (50 mL) and washed with NaHCO₃ (30 mL). The organic layer was dried over anhydrous Na₂SO₄, filtered, and concentrated under reduced pressure. The residue was purified by silica gel chromatography, eluting with DCM: MeOH (1:0-10:1, v/v), to give SW-II-118 (105 mg, 61%) as a yellow oil.

LCMS: Rt: 1.946 min; MS m/z (ELSD): 744.4 [M+H]⁺;

HPLC: 99.64% purity at ELSD; RT=5.875 min.

¹H NMR (400 MHz, CDCl₃) δ 7.11 (s, 4H), 4.91-4.79 (m, 1H), 4.26 (t, J=7.2 Hz, 2H), 3.80-3.68 (m, 2H), 2.90 (t, J=7.1 Hz, 4H), 2.81-2.67 (m, 4H), 2.62-2.52 (m, 2H), 2.28 (td, J=7.5, 2.6 Hz, 4H), 1.64-1.51 (m, 11H), 1.38-1.17 (m, 42H), 0.93-0.82 (m, 9H).

¹³C NMR (101 MHz, CDCl₃) δ 173.61 (d, J=11.7 Hz), 141.11 (s), 134.90 (s), 128.74 (s), 128.51 (s), 77.40 (s), 77.08 (s), 76.77 (s), 74.17 (s), 64.90 (s), 57.48 (s), 56.24 (s), 53.98 (s), 35.25 (s), 34.66 (d, J=14.4 Hz), 34.16 (d, J=5.1 Hz), 33.67 (s), 31.86 (s), 29.52 (d, J=2.4 Hz), 29.24 (s), 29.21-28.74 (m), 26.90 (d, J=4.9 Hz), 25.42-24.92 (m), 24.92-24.88 (m), 24.74 (s), 22.67 (s), 22.37 (s), 14.04 (d, J=15.7 Hz).

### C. Compound SW-II-120

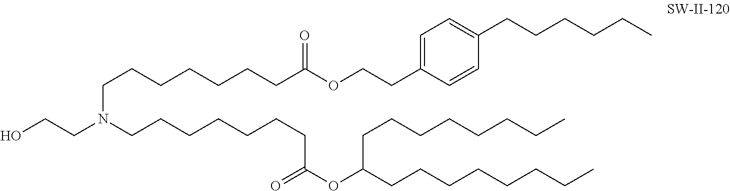 [SMILES: CCCCCCCCC(CCCCCCCC)OC(=O)CCCCCCCN(CCO)CCCCCCCC(=O)OCCc1ccc(CCCCCC)cc1]

### 1. Synthesis of Intermediate 3

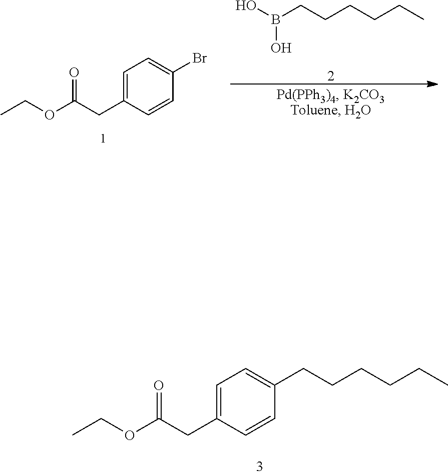

A mixed solution of compound 1 (1.22 g, 5.0 mmol, 1.0 eq.), compound 2 (1.30 mg, 10.0 mmol, 2.0 eq.), Pd(PPh₃)₄ (289 mg, 0.25 mmol, 0.05 eq.) and K₂CO₃ (1.38 g, 10.0 mmol, 2.0 eq.) in toluene (10 ml) and H₂O (1 ml) was stirred at 110° C. under N₂ for 1 hour. TLC (petroleum ether:ethyl acetate=19:1) showed that compound 1 was consumed and a new spot was observed. The reaction mixture was diluted with DCM (50 mL) and washed with H₂O (40 mL), dried over anhydrous Na₂SO₄, filtered and concentrated under reduced pressure. The residue was purified by silica gel chromatography, eluting with petroleum ether: ethyl acetate (1:0-10:1), to obtain compound 3 (0.78 g, 62%) as a colorless oil.

¹H NMR (400 MHz, CDCl₃) δ 7.19 (d, J=8.1 Hz, 2H), 7.13 (d, J=8.1 Hz, 2H), 4.14 (q, J=7.1 Hz, 2H), 3.57 (s, 2H), 2.62-2.51 (m, 2H), 1.58 (d, J=11.1 Hz, 2H), 1.35-1.21 (m, 9H), 0.88 (t, J=6.7 Hz, 3H).

### 2. Synthesis of Intermediate 4

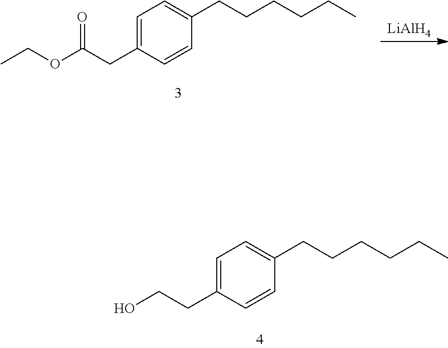 [SMILES: CCCCCCc1ccc(CC(=O)OCC)cc1 ; "LiAlH4" ; CCCCCCc1ccc(CCO)cc1]

LiAlH₄ (477 mg, 12.56 mmol, 4.0 eq.) was added to a solution of compound 3 (780 mg, 3.14 mmol, 1.0 eq.) in THF (10 mL) at −78° C., and then the reaction was stirred at 10° C. for 3 hours. TLC showed that the reaction went well. The reaction was concentrated, diluted with Na₂SO₄ (20 mL) and extracted with EA (30 mL×2). The organic phase was dried over anhydrous Na₂SO₄, filtered and concentrated under reduced pressure to obtain compound 4 (640 mg, crude product) as a colorless oil.

### 3. Synthesis of Intermediate 6

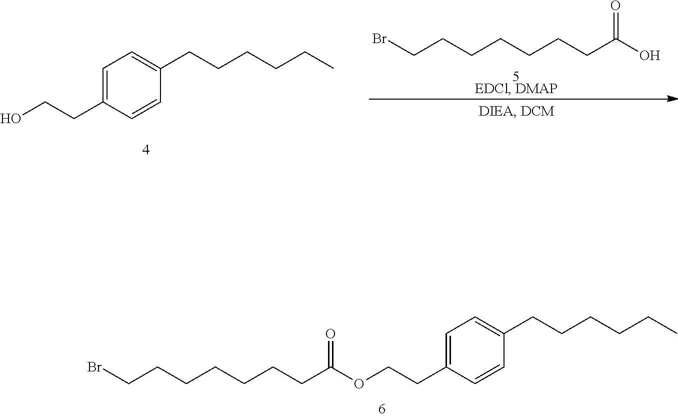

A solution of compound 4 (640 mg, 3.10 mmol, 1.0 eq.), compound 5 (1.06 g, 4.70 mmol, 1.5 eq.), EDCI (2.98 g, 15.5 mmol, 5.0 eq.), DMAP (1.85 g, 15.0 eq.), and DIEA (4.0 g, 31.0 mmol, 10.0 eq.) in DCM (10 mL) was stirred at 10° C. under N₂ for 16 h. TLC (DCM:MeOH=10:1) showed that the reaction was completed and new major spots were observed. The mixture was concentrated under reduced pressure, and the residue was purified by silica gel chromatography, eluting with PE:EA (1:0-10:1, v/v), to give compound 6 (465 mg, 36%) as a colorless oil.

### 4. Synthesis of the Final Product SW-II-120

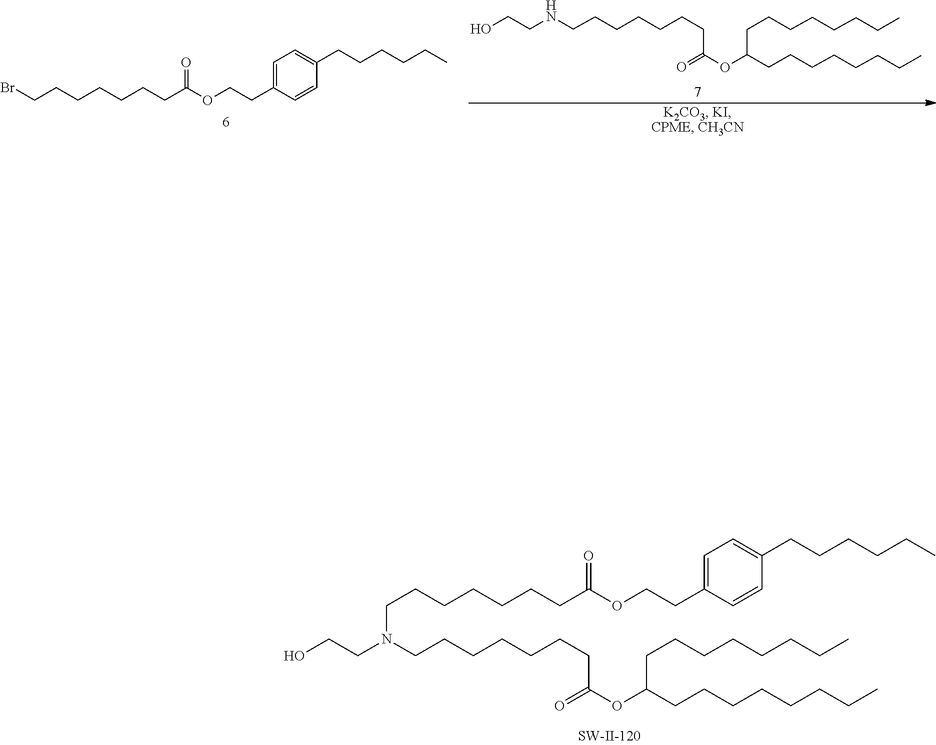

The mixture of compound 6 (100 mg, 0.25 mmol, 1.0 eq.), compound 7 (161 mg, 0.36 mmol, 1.5 eq.), K₂CO₃ (104 mg, 0.75 mmol, 3.0 eq.) and KI (83 mg, 0.50 mmol, 2.0 eq.) in CPME (1 mL) and CH₃CN (1 mL) was stirred at 90° C. under N₂ for 16 hours. The reaction mixture was concentrated under reduced pressure, and the residue was diluted with EtOAc (50 mL) and washed with NaHCO₃ (30 mL). The organic layer was dried over anhydrous Na₂SO₄, filtered, and concentrated under reduced pressure. The residue was purified by silica gel chromatography, eluting with DCM: MeOH (1:0-10:1, v/v), to give SW-II-120 (100 mg, 52%) as a yellow oil.

LCMS: Rt: 2.500 min; MS m/z (ELSD): 772.4 [M+H]⁺;

HPLC: 99.70% purity at ELSD; RT=8.675 min.

¹H NMR (400 MHz, CDCl₃) δ 7.07 (d, J=8.9 Hz, 4H), 4.89-4.73 (m, 1H), 4.23 (t, J=7.2 Hz, 2H), 3.83-3.65 (m, 2H), 2.87 (t, J=7.2 Hz, 4H), 2.82-2.67 (m, 4H), 2.61-2.45 (m, 2H), 2.25 (td, J=7.5, 2.5 Hz, 4H), 1.65-1.44 (m, 15H), 1.27 (dd, J=13.2, 11.3 Hz, 42H), 0.85 (t, J=6.8 Hz, 9H).

¹³C NMR (101 MHz, CDCl₃) δ 173.57 (d, J=11.5 Hz), 141.13 (s), 134.88 (s), 128.73 (s), 128.48 (s), 77.45 (s), 77.13 (s), 76.81 (s), 74.14 (s), 64.89 (s), 57.34 (s), 56.17 (s), 53.92 (s), 35.57 (s), 34.64 (d, J=16.1 Hz), 34.14 (d, J=3.3 Hz), 31.79 (d, J=13.4 Hz), 31.49 (s), 29.50 (d, J=2.2 Hz), 29.23 (s), 29.10-28.71 (m), 26.85 (d, J=5.0 Hz), 25.49-25.38 (m), 25.13 (d, J=35.4 Hz), 24.72 (s), 22.63 (d, J=5.8 Hz), 14.11 (s).

### D. Compound SW-II-121

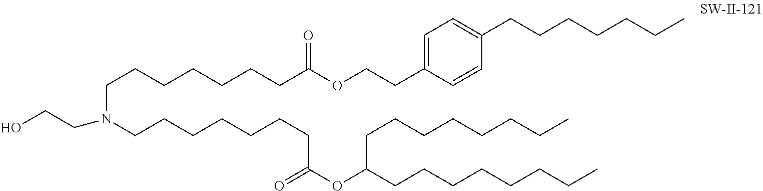 [SMILES: CCCCCCCCC(CCCCCCCC)OC(=O)CCCCCCCN(CCO)CCCCCCCC(=O)OCCc1ccc(CCCCCCC)cc1]

### 1. Synthesis of Intermediate 3

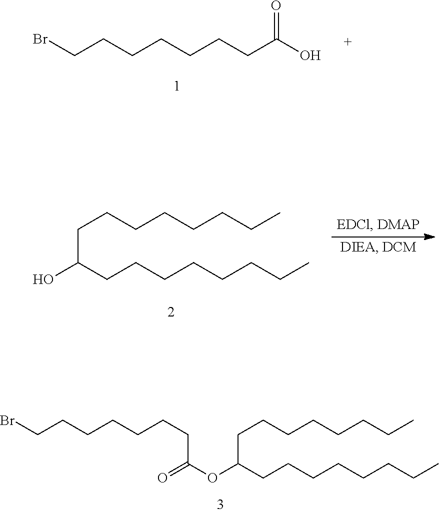 [SMILES: O=C(O)CCCCCCCBr ; CCCCCCCCC(O)CCCCCCCC ; "DIEA, DCM" ; "EDCl, DMAP" ; CCCCCCCCC(CCCCCCCC)OC(=O)CCCCCCCBr]

To a solution of compound 1 (1.3 g, 5.86 mmol, 1.5 eq.) and compound 2 (1 g, 3.9 mmol, 1.0 eq.) in DCM (20 mL) was added EDCI (1.495 g, 7.8 mmol, 2.0 eq.), DMAP (0.19 g, 1.56 mmol, 0.4 eq.), and DIEA (2.57 mL, 15.6 mmol, 4.0 eq.). The reaction mixture was stirred at room temperature under N₂ for 16 hours. TLC (petroleum ether:ethyl acetate=19:1) showed that compound 2 was consumed and the desired product was formed. The reaction mixture was diluted with DCM (20 mL) and washed with H₂O (40 mL), dried over anhydrous Na₂SO₄, filtered and concentrated under reduced pressure. The residue was purified by silica gel chromatography, eluting with petroleum ether:ethyl acetate (1:0-10:1), to give compound 3 (1.2 g, 66.9%) as a yellow oil.

¹H NMR (400 MHz, CDCl₃) δ 4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Intermediate 5

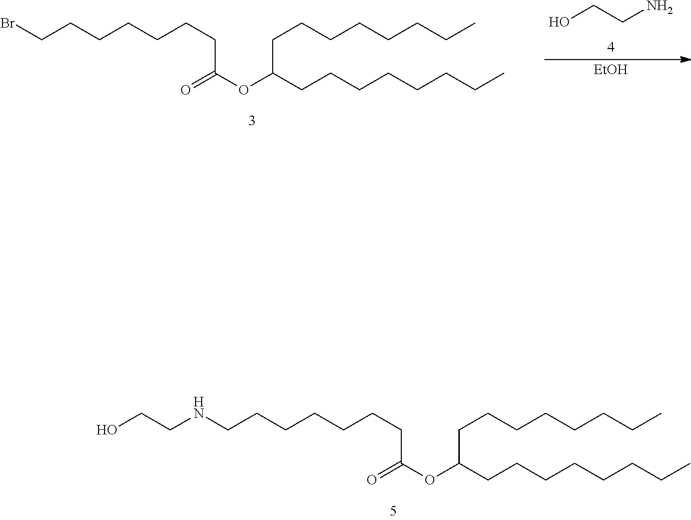

A solution of compound 3 (5.2 g, 11.30 mmol, 1.0 eq.) and compound 4 (20.6 g, 339 mmol, 30 eq.) in EtOH (5 mL) was stirred at 60° C. under N₂ for 16 hours. TLC (petroleum ether:ethyl acetate=19:1) showed that compound 3 was consumed and TLC (DCM/MeOH=10/1) showed that new major spots were observed. The reaction mixture was concentrated under reduced pressure, and the residue was diluted with EtOAc (50 mL) and washed with H₂O (3×50 mL). The organic layer was dried over anhydrous Na₂SO₄, filtered and concentrated under reduced pressure. The residue was purified by silica gel chromatography, eluting with DCM: MeOH (1:0-10:1, v/v), to give compound 5 (3 g, 60%).

### 3. Synthesis of Intermediate 8

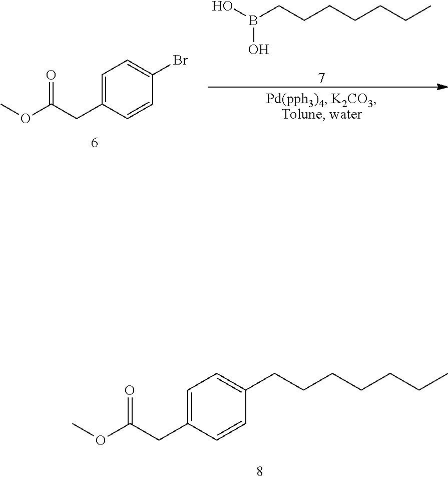

To a mixed solution of compound 6 (1 g, 4.115 mmol, 1 eq.) and compound 7 (889 mg, 6.173 mmol, 1.5 eq) in Toluene/water (10 mL/1 mL) was added Pd(pph₃)₄ (238 mg, 0.206 mmol, 0.05 eq.) and K₂CO₃ (1.7 g, 12.35 mmol, 3 eq). The mixture was stirred at 110° C. under N₂ for 2 hours. TLC (PE:EA=10:1) showed that the reaction was completed and new major spots were observed. The mixture was extracted with EA and washed with water. The organic layer was dried over anhydrous Na₂SO₄, filtered, and concentrated in vacuo. The residue was purified by silica gel chromatography, eluting with PE:EA (1:0-10:1), to obtain compound 8 (714 mg, 66%) as a colorless oil.

### 4. Synthesis of Intermediate 9

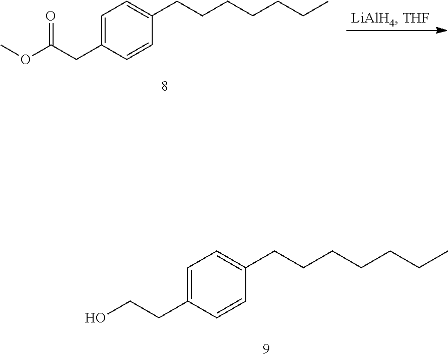

To a solution of compound 8 (714 mg, 2.725 mmol, 1 eq.) in THF (7 mL) was added LiAlH₄ (2.7 mL, 2.725 mmol, 1M, in THF, 1 eq.) at 0° C. under N₂, and then the reaction was stirred at room temperature for 2 hours. TLC (PE:EtOAc=10:1) showed that the reaction was completed and new major spots were observed. The mixture was quenched with water (2.7 mL) and treated with 2N HCl to adjust the pH between 6 and 7, extracted with EA and washed with brine. The organic layer was dried over anhydrous Na₂SO₄, filtered, and concentrated in vacuo. The residue was purified by silica gel chromatography, eluting with PE:EA (1:0-10:1), to give compound 9 (103 mg, 63%) as a colorless oil.

### 5. Synthesis of Intermediate 11

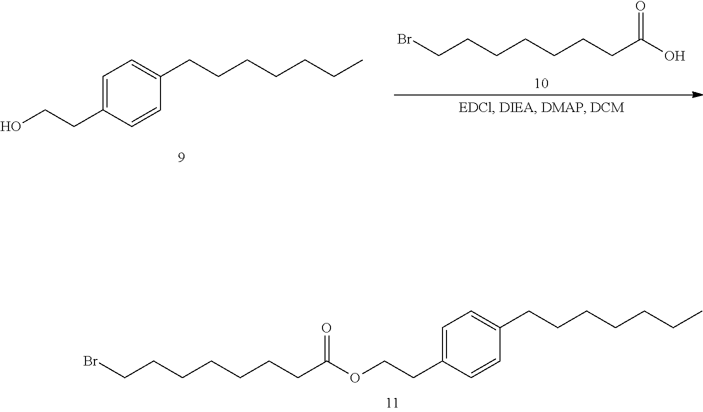

To a DCM (3 mL) solution containing compound 9 (300 mg, 1.364 mmol, 1 eq.) and compound 10 (363 mg, 1.64 mmol, 1.2 eq.) was added EDCI (524 mg, 2.728 mmol, 2 eq.), DMAP (67 mg, 0.546 mmol, 0.4 eq.), and DIEA (704 mg, 5.456 mmol, 4 eq.). The reaction mixture was stirred at room temperature under N₂ for 16 hours. TLC (petroleum ether: ethyl acetate=10:1) showed that the desired product was formed. The reaction mixture was extracted with EA and washed with water. The organic layer was dried over anhydrous Na₂SO₄, filtered, and concentrated in vacuo. The residue was purified by silica gel chromatography, eluting with petroleum ether:ethyl acetate (1:0-10:1), to give compound 11 (169 mg, 29%) as a colorless oil.

### 6. Synthesis of the Final Product SW-II-121

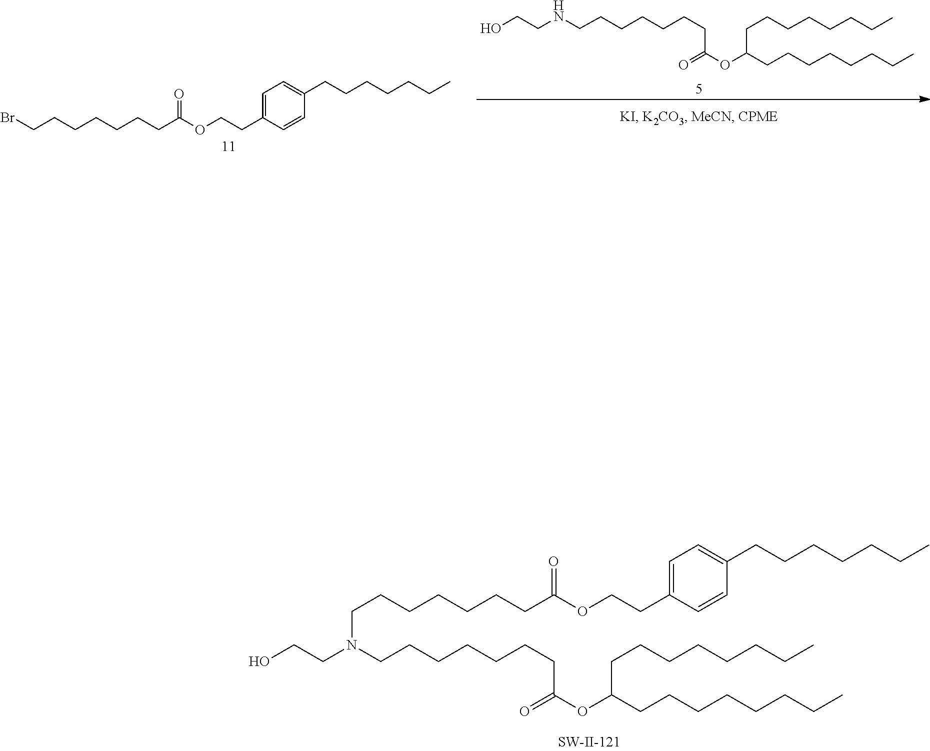

To a CPME/CH₃CN (2 mL/2 mL) mixed solvent containing compound 11 (169 mg, 0.399 mmol, 1 eq.) and compound 5 (176 mg, 0.399 mmol, 1 eq.) was added K₂CO₃ (330 mg, 2.394 mmol, 6 eq.) and KI (132 mg, 0.798 mmol, 2 eq.). After the addition was completed, the mixture was stirred overnight at 90° C. under N₂. TLC (DCM:MeOH=10:1) showed that the reaction was completed and new major spots were observed. The mixture was extracted with EA and washed with water. The organic layer was dried over anhydrous Na₂SO₄, filtered and concentrated under reduced pressure. The residue was purified by silica gel chromatography, eluting with DCM: MeOH (1:0-10:1, v/v), to give compound SW-II-121 (145 mg, 46%) as a yellow oil.

LCMS: Rt: 1.493 min; MS m/z (ELSD): 786.5 [M+H]⁺;

HPLC: 99.869% purity at ELSD; RT=10.655 min.

¹H NMR (400 MHz, CDCl₃) δ 7.11 (s, 4H), 4.92-4.80 (m, 1H), 4.26 (t, J=7.2 Hz, 2H), 3.80 (s, 2H), 2.87 (dd, J=26.6, 19.4 Hz, 7H), 2.62-2.51 (m, 2H), 2.28 (td, J=7.2, 3.6 Hz, 4H), 1.75-1.45 (m, 14H), 1.42-1.09 (m, 45H), 0.88 (t, J=6.8 Hz, 9H).

¹³C NMR (101 MHz, CDCl₃) δ 173.61 (d, J=12.3 Hz), 141.20 (s), 134.90 (s), 128.75 (s), 128.51 (s), 77.35 (s), 77.03 (s), 76.72 (s), 74.21 (s), 64.93 (s), 54.15 (s), 35.59 (s), 34.66 (d, J=16.6 Hz), 34.16 (d, J=3.0 Hz), 31.85 (d, J=4.4 Hz), 31.55 (s), 29.64-29.15 (m), 29.15-28.78 (m), 26.85 (d, J=4.5 Hz), 25.33 (s), 24.95 (s), 24.72 (s), 22.68 (s), 14.12 (s).

### E. Compound SW-II-122

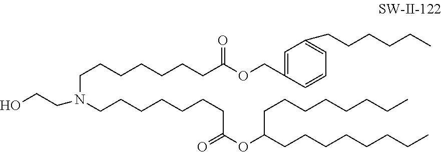 [SMILES: CCCCCCCCC(CCCCCCCC)OC(=O)CCCCCCCN(CCO)CCCCCCCC(=O)OCc1cccc(CCCCCC)c1]

### 1. Synthesis of Compound 3

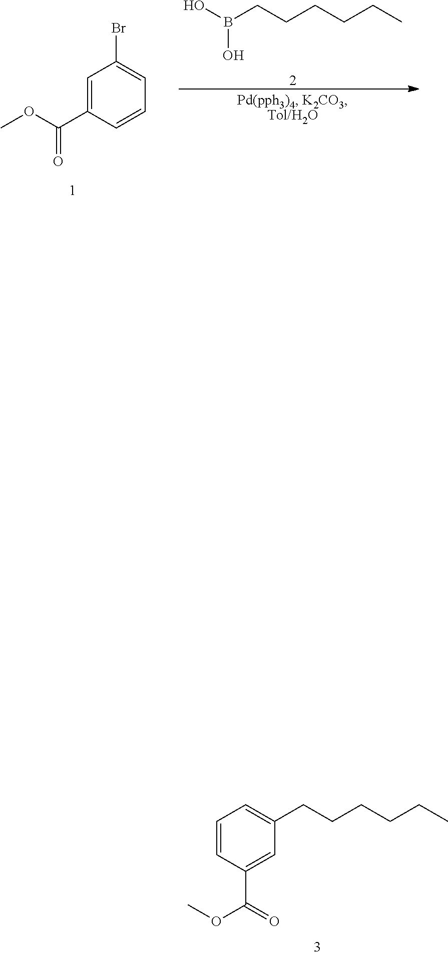

Compound 1 (1 g, 4.65 mmol, 1 eq.) and Compound 2 (726 mg, 5.58 mmol, 1.2 eq.) were dissolved in toluene/water (10/1, 20 mL), and then K₂CO₃ (1.92 g, 13.9 mmol, 3 eq.) and Pd(pph₃)₄ (269 mg, 0.23 mmol, 0.05 eq) were added. The reaction mixture was heated to 110° C. under N₂ and stirred for 2 hours. TLC (petroleum ether/ethyl acetate=19/1) showed that compound 1 was consumed and new major spots were observed. The reaction mixture was quenched with H₂O (80 mL) and extracted with ethyl acetate (60 mL×3). The organic layer was dried over anhydrous Na₂SO₄, filtered, and concentrated under reduced pressure. The residue was purified by silica gel chromatography, eluting with petroleum ether/ethyl acetate (1/0-10/1), to give compound 3 (800 mg, 78%) as a yellow oil.

### 2. Synthesis of Compound 4

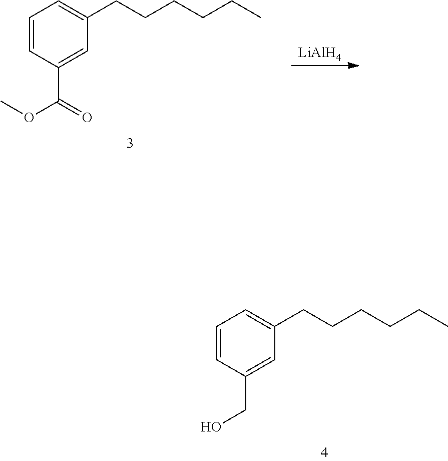 [SMILES: CCCCCCc1cccc(C(=O)OC)c1 ; "LiAlH4" ; CCCCCCc1cccc(CO)c1]

LiAlH₄ (3.2 mL, 3.18 mmol, 1 eq) was added to a solution of compound 3 (700 mg, 3.18 mmol, 1.0 eq.) in THF (14 mL) at 0° C. under N₂. The reaction was allowed to warm to room temperature and stirred under nitrogen for 2 hours. TLC (PE/EtOAc=10/1) showed that the reaction was completed and new major spots were observed. The mixture was quenched with water (3.2 mL) and 1M HCl (3.2 mL), respectively. To the mixture was added water (6 mL), and then extracted with ethyl acetate (60 mL×3). The organic layer was washed with brine (30 mL×2), dried over anhydrous Na₂SO₄, filtered, and concentrated under reduced pressure. The residue was purified by silica gel chromatography, eluting with ethyl acetate/petroleum ether=1/10, to obtain compound 4 (600 mg, 98%) as a yellow oil.

### 3. Synthesis of Compound 6

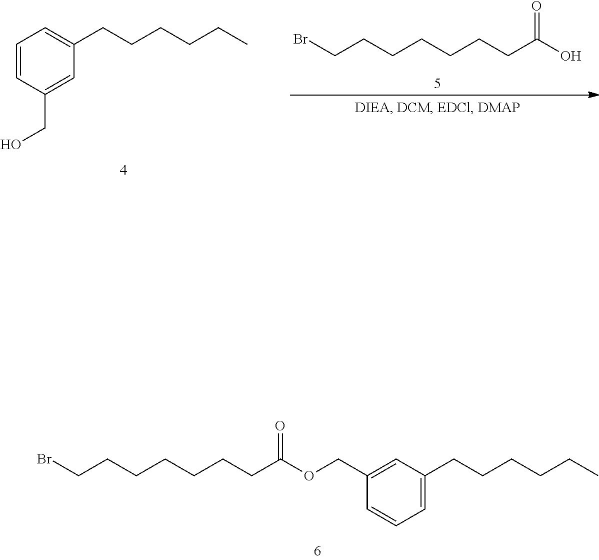

Compound 4 (680 mg, 3.5 mmol, 1.0 eq.) and Compound 5 (1.13 g, 5.1 mmol, 1.5 eq.) were dissolved in DCM (10 mL), and then EDCI (1.20 g, 6.25 mmol, 2.0 eq.), DMAP (166 mg, 1.36 mmol, 0.4 eq.) and DIEA (1.78 g, 13.8 mmol, 4.0 eq.) were added therein. After the addition, the reaction mixture was stirred overnight at room temperature under nitrogen. TLC (DCM/MeOH=30/1) showed that the starting material was consumed and a new spot was formed. The mixture was quenched with water (70 mL) and extracted with DCM (80 mL×3). The combined organic layer was washed with brine (2×20 mL), dried over anhydrous Na₂SO₄, filtered and concentrated under reduced pressure. The residue was purified by silica gel chromatography, eluting with ethyl acetate/petroleum ether=3/97 solution to obtain compound 6 (680 mg, 48.5%) as a yellow oil.

### 4. Synthesis of SW-II-122

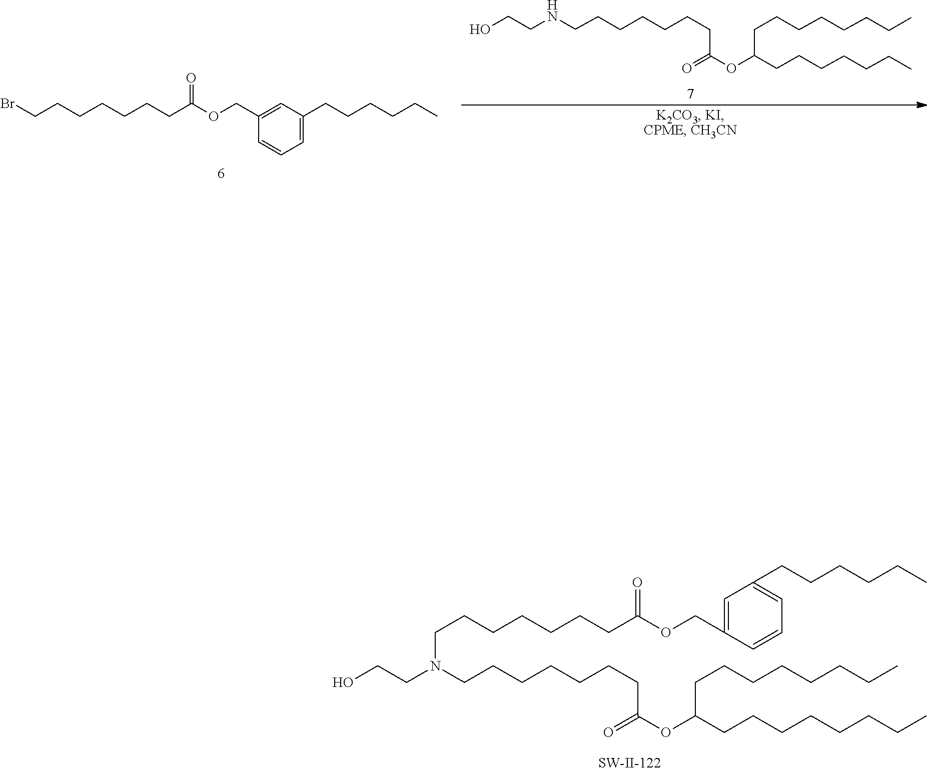

Compound 6 (108 mg, 0.27 mmol, 1.2 eq) and compound 7 (100 mg, 0.23 mmol, 1 eq.) were dissolved in CPME (2 mL) and CH₃CN (2 mL), and then potassium carbonate (157 mg, 1.14 mmol, 5.0 eq) and potassium iodide (75 mg, 0.45 mmol, 2.0 eq) were added therein. After the addition, the reaction mixture was stirred at 90° C. under nitrogen for 16 hours. TLC (DCM/MeOH=10/1) showed that the reaction was completed. The reaction mixture was concentrated under reduced pressure. The residue was purified by silica gel chromatography, eluting with DCM/MeOH (1/0-10:1, v/v), to obtain SW-II-122 (68 mg, 40%) as a colorless oil.

LCMS: Rt: 1.487 min; MS m/z (ELSD): 758.5 [M+H]⁺;

HPLC: 97.3% purity at ELSD; RT=7.622 min.

¹H NMR (400 MHz, CDCl₃) δ 7.32 (d, J=26.4 Hz, 1H), 7.17 (dd, J=27.2, 21.1 Hz, 3H), 5.09 (s, 2H), 4.91-4.79 (m, 1H), 3.85 (s, 2H), 2.98 (s, 2H), 2.87 (s, 4H), 2.65-2.54 (m, 2H), 2.35 (t, J=7.6 Hz, 2H), 2.28 (t, J=7.6 Hz, 2H), 1.74-1.57 (m, 9H), 1.50 (d, J=5.6 Hz, 4H), 1.37-1.15 (m, 43H), 0.94-0.80 (m, 9H).

¹³C NMR (101 MHz, CDCl₃) δ 173.55 (d, J=2.4 Hz), 143.35 (s), 135.92 (s), 128.67-128.19 (m), 125.47 (s), 77.36 (s), 77.04 (s), 76.73 (s), 74.22 (s), 66.27 (s), 57.15 (s), 56.74 (s), 54.14 (s), 35.88 (s), 34.55 (s), 34.15 (d, J=3.6 Hz), 31.79 (d, J=15.2 Hz), 31.43 (s), 29.52 (d, J=2.8 Hz), 29.25 (s), 28.92 (dd, J=14.2, 5.8 Hz), 26.77 (d, J=4.8 Hz), 25.33 (s), 24.92 (s), 24.71 (s), 24.48 (s), 22.64 (d, J=6.8 Hz), 14.12 (s).

### F. Compound SW-II-127

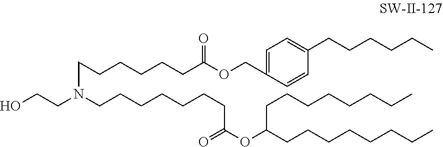 [SMILES: CCCCCCCCC(CCCCCCCC)OC(=O)CCCCCCCN(CCO)CCCCCCC(=O)OCc1ccc(CCCCCC)cc1]

### 1. Synthesis of Compound 3

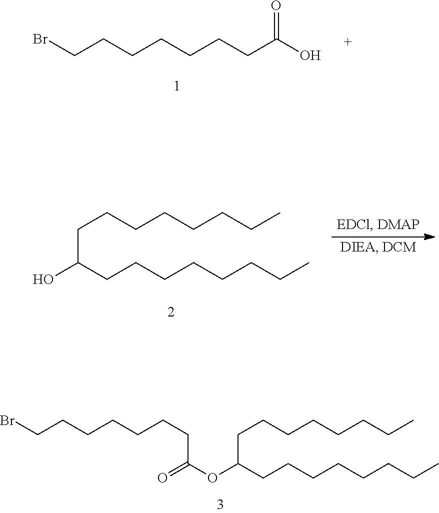 [SMILES: O=C(O)CCCCCCCBr ; CCCCCCCCC(O)CCCCCCCC ; "DIEA, DCM" ; "EDCl, DMAP" ; CCCCCCCCC(CCCCCCCC)OC(=O)CCCCCCCBr]

Compound 1 (1.3 g, 5.86 mmol, 1.5 eq.) and Compound 2 (1 g, 3.9 mmol, 1.0 eq.) were dissolved in DCM (20 mL), and then EDCI (1.495 g, 7.8 mmol, 2.0 eq.), DMAP (0.19 g, 1.56 mmol, 0.4 eq.) and DIEA (2.57 mL, 15.6 mmol, 4.0 eq.) were added therein. The reaction mixture was stirred at room temperature under nitrogen for 16 hours. TLC (petroleum ether/ethyl acetate=19/1) showed that compound 2 was consumed and the desired product was formed. The reaction mixture was diluted with DCM (20 mL) and washed with H₂O (40 mL), dried over anhydrous Na₂SO₄, filtered and concentrated under reduced pressure. The residue was purified by silica gel chromatography, eluting with petroleum ether/ethyl acetate (1/0-10/1), to give compound 3 (1.2 g, 66.9%) as a yellow oil.

¹H NMR (400 MHz, CDCl₃) δ 4.92-4.82 (m, 1H), 3.42 (t, J=6.8 Hz, 2H), 2.31 (t, J=7.5 Hz, 2H), 1.95-1.82 (m, 2H), 1.70-1.19 (m, 36H), 0.90 (t, J=6.8 Hz, 6H).

### 2. Synthesis of Compound 5

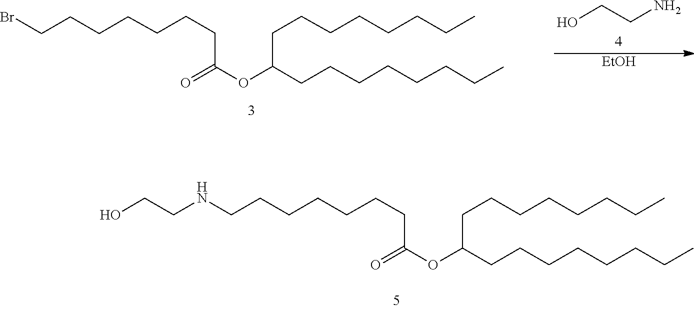

Compound 3 (5.2 g, 11.30 mmol, 1.0 eq.) and compound 4 (20.6 g, 339 mmol, 30 eq.) were added to EtOH (5 mL), and then the mixture was stirred at 60° C. under nitrogen for 16 hours. TLC (petroleum ether/ethyl acetate=19/1) showed that compound 3 was consumed and TLC (DCM/MeOH=10/1) showed that new major spots were observed. The reaction mixture was concentrated under reduced pressure. The residue was diluted with EtOAc (50 mL) and washed with H₂O (3×50 mL). The organic layer was dried over anhydrous Na₂SO₄, filtered, and concentrated under reduced pressure. The residue was purified by silica gel chromatography, eluting with DCM/MeOH (1/0-10:1, v/v), to give compound 5 (3 g, 60%) as a yellow oil.

¹H NMR (400 MHz, CDCl₃) δ 4.95-4.75 (m, 1H), 3.74-3.58 (m, 2H), 2.87-2.74 (m, 2H), 2.69-2.56 (m, 2H), 2.36 (s, 2H), 2.28 (t, J=7.5 Hz, 2H), 1.65-1.42 (m, 8H), 1.38-1.17 (m, 30H), 0.88 (t, J=6.8 Hz, 6H).

### 3. Synthesis of Compound 8

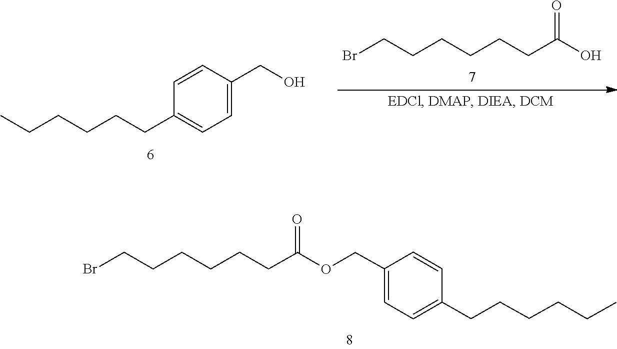 [SMILES: O=C(O)CCCCCCBr ; CCCCCCc1ccc(CO)cc1 ; CCCCCCc1ccc(COC(=O)CCCCCCBr)cc1]

Compound 7 (522 mg, 2.5 mmol, 1.2 eq.) and compound 6 (400 mg, 2.083 mmol, 1 eq.) were dissolved in DCM (4 mL), and then EDCI (800 mg, 4.166 mmol, 2 eq.), DMAP (102 mg, 0.833 mmol, 0.4 eq.) and DIEA (1.075 mg, 8.332 mmol, 4 eq.) were added therein. After the addition, the reaction mixture was stirred overnight at room temperature under nitrogen. TLC (PE:EA=10:1) showed that the starting material was consumed and a new spot was formed. The reaction mixture was concentrated under reduced pressure. The residue was purified by silica gel chromatography, eluting with PE/EA (1/0-10/1), to give compound 8 (454 mg, 57%) as a colorless oil.

### 4. Synthesis of SW-II-127

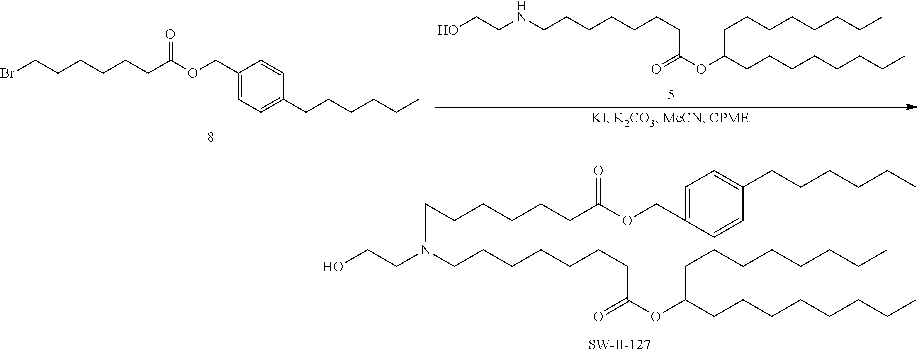 [SMILES: CCCCCCc1ccc(COC(=O)CCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCC(=O)OCc1ccc(CCCCCC)cc1]

Compound 8 (100 mg, 0.262 mmol, 1 eq.) and compound 5 (139 mg, 0.314 mmol, 1.2 eq.) were dissolved in CPME/CH₃CN (1 mL/1 mL), and then potassium carbonate (217 mg, 1.572 mmol, 6 eq.) and potassium iodide (87 mg, 0.524 mmol, 2 eq.) were added therein. After the addition, the reaction mixture was stirred at 90° C. overnight under nitrogen. TLC (DCM/MeOH=10/1) showed that the reaction was completed and the desired product was formed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-127 (42.49 mg, 22%) as a yellow oil.

LCMS: Rt: 1.323 min; MS m/z (ELSD): 744.5 [M+H]⁺;

HPLC: 99.742% purity at ELSD; RT=7.339 min.

¹H NMR (400 MHz, CDCl₃) δ 7.25 (s, 2H), 7.17 (d, J=8.0 Hz, 2H), 5.07 (s, 2H), 4.91-4.82 (m, 1H), 3.83 (s, 2H), 2.90 (d, J=44.8 Hz, 5H), 2.64-2.55 (m, 2H), 2.35 (t, J=7.4 Hz, 2H), 2.28 (t, J=7.5 Hz, 2H), 1.76-1.46 (m, 14H), 1.42-1.19 (m, 41H), 0.88 (t, J=6.8 Hz, 9H).

¹³C NMR (101 MHz, CDCl₃) δ 173.50 (d, J=8.5 Hz), 133.17 (s), 128.61 (s), 128.34 (s), 77.29 (d, J=11.4 Hz), 77.03 (s), 76.71 (s), 74.23 (s), 66.19 (s), 54.20 (s), 35.71 (s), 34.56 (s), 34.10 (d, J=8.8 Hz), 31.80 (d, J=15.4 Hz), 31.43 (s), 29.53 (d, J=2.5 Hz), 29.25 (s), 28.95 (d, J=10.5 Hz), 28.63 (s), 26.71 (d, J=18.2 Hz), 25.33 (s), 24.93 (s), 24.62 (s), 22.65 (d, J=6.6 Hz), 14.13 (s).

### G. Compound SW-II-134-1

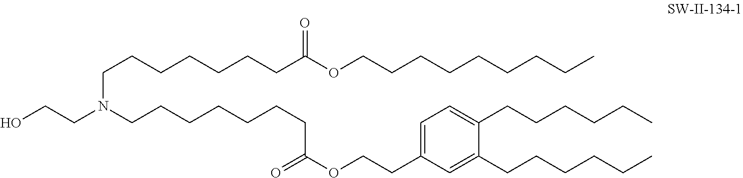 [SMILES: CCCCCCCCCOC(=O)CCCCCCCN(CCO)CCCCCCCC(=O)OCCc1ccc(CCCCCC)c(CCCCCC)c1]

### 1. Synthesis of Compound 3

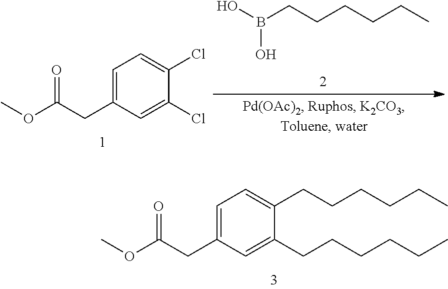

To a mixture of compound 1 (500 mg, 2.283 mmol, 1 eq.) and compound 2 (890 mg, 6.849 mmol, 3 eq) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq). The mixture was stirred at 110° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to give compound 3 (723 mg, 99.6%) as a colorless oil.

### 2. Synthesis of Compound 4

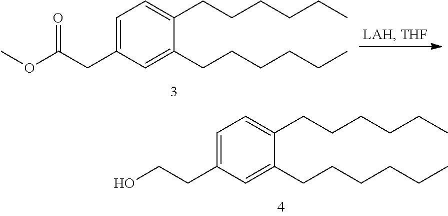 [SMILES: CCCCCCc1ccc(CC(=O)OC)cc1CCCCCC ; CCCCCCc1ccc(CCO)cc1CCCCCC]

To a mixture of compound 3 (723 mg, 2.27 mmol, 1 eq.) in THF (8 mL) was added lithium aluminum hydride (2.3 mL, 2.27 mmol, 1M in THF, 1 eq.) under nitrogen at 0° C. The mixture was stirred at room temperature for 3 hours. TLC (PE/EA=5/1) indicated that the reaction was completed and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to give compound 4 (381 mg, 58%) as a colorless oil, which was used without further purification.

### 3. Synthesis of Compound 6

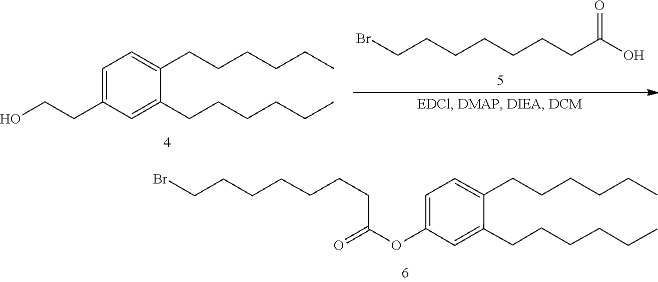

To a mixture of compound 4 (381 mg, 1.3 mmol, 1 eq.) and compound 5 (352 mg, 1.6 mmol, 1.2 eq.) in DCM (4 mL) was added EDCI (499 mg, 2.6 mmol, 2 eq.) and DMAP (63 mg, 0.52 mmol, 0.4 eq.). Then DMA (671 mg, 5.2 mmol, 4 eq.) was added. The reaction mixture was stirred at room temperature under nitrogen for 16 hours. TLC (petroleum ether/ethyl acetate=20/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (272 mg, 44%) as a colorless oil.

### 4. Synthesis of SW-II-134-1

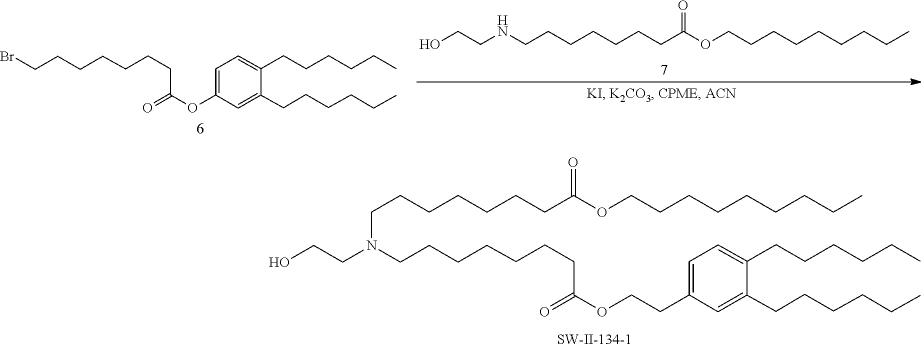

To the mixture of compound 6 (150 mg, 0.303 mmol, 1 eq.) and compound 7 (110 mg, 0.333 mmol, 1.1 eq.) in CPME/CH₃CN (2 mL/2 mL) was added potassium carbonate (251 mg, 1.818 mmol, 6 eq.) and potassium iodide (101 mg, 0.61 mmol, 2 eq.). After the addition, the mixture was stirred at 90° C. overnight under nitrogen. TLC (DCM/MeOH=15/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-134-1 (168 mg, 75%) as a yellow oil.

LCMS: Rt: 1.276 min; MS m/z (ELSD): 744.4 [M+H]⁺;

HPLC: 98.481% purity at ELSD; RT=10.724 min.

¹H NMR (400 MHz, CDCl₃) δ 7.06 (d, J=7.6 Hz, 1H), 7.01-6.93 (m, 2H), 4.25 (t, J=7.3 Hz, 2H), 4.05 (t, J=6.8 Hz, 2H), 3.85-3.72 (m, 2H), 2.98-2.69 (m, 8H), 2.62-2.48 (m, 4H), 2.29 (t, J=7.5 Hz, 4H), 1.72-1.48 (m, 14H), 1.45-1.17 (m, 36H), 0.89 (dt, J=11.9, 6.0 Hz, 9H).

¹³CNMR (101 MHz, CDCl₃) δ 173.78 (d, J=16.7 Hz), 140.72 (s), 138.81 (s), 134.91 (s), 129.70 (s), 129.22 (s), 126.19 (s), 77.30 (d, J=11.4 Hz), 77.03 (s), 76.72 (s), 65.02 (s), 64.49 (s), 57.42 (s), 56.36 (s), 54.08 (s), 34.76 (s), 34.22 (d, J=4.2 Hz), 32.74 (s), 32.36 (s), 31.81 (d, J=9.1 Hz), 31.35 (d, J=5.3 Hz), 29.49 (d, J=2.8 Hz), 29.24 (d, J=2.2 Hz), 28.92 (s), 28.66 (s), 26.86 (s), 25.93 (s), 25.04 (s), 24.78 (d, J=6.6 Hz), 22.65 (d, J=2.6 Hz), 14.10 (s).

### H. Compound SW-II-134-2

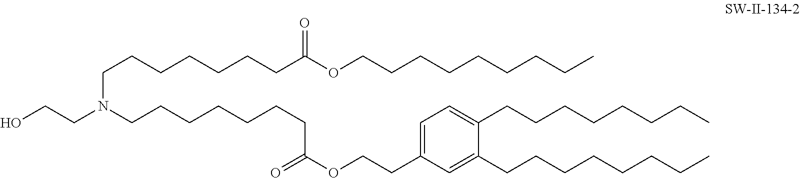 [SMILES: CCCCCCCCCOC(=O)CCCCCCCN(CCO)CCCCCCCC(=O)OCCc1ccc(CCCCCCCC)c(CCCCCCCC)c1]

### 1. Synthesis of Compound 3

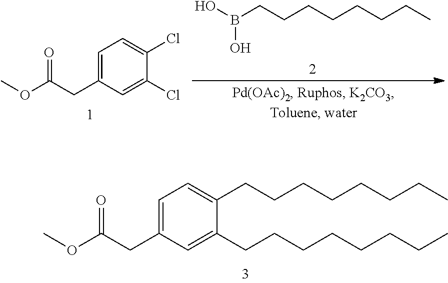 [SMILES: COC(=O)Cc1ccc(Cl)c(Cl)c1 ; CCCCCCCCB(O)O ; CCCCCCCCc1ccc(CC(=O)OC)cc1CCCCCCCC]

To a mixture of compound 1 (500 mg, 2.283 mmol, 1 eq.) and compound 2 (1.08 g, 6.849 mmol, 3 eq.) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq). The mixture was stirred at 110° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to give compound 3 (854 mg, 100%) as a colorless oil.

### 2. Synthesis of Compound 4

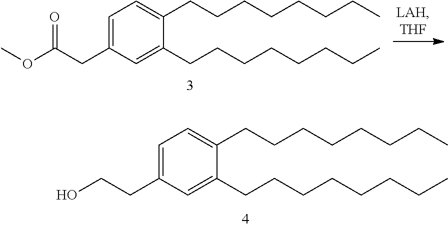 [SMILES: CCCCCCCCc1ccc(CC(=O)OC)cc1CCCCCCCC ; CCCCCCCCc1ccc(CCO)cc1CCCCCCCC]

To a mixture of compound 3 (854 mg, 2.28 mmol, 1 eq.) in THF (9 mL) was added lithium aluminum hydride (2.3 mL, 2.28 mmol, 1 M in THF, 1 eq.) at 0° C. under N₂. The mixture was stirred at room temperature for 3 hours. TLC (PE/EA=5/1) indicated that the reaction was completed and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 4 (724 mg, 92%) as a colorless oil, which was used without further purification.

### 3. Synthesis of Compound 6

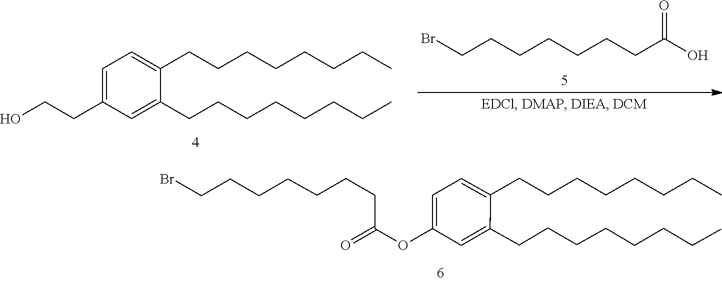 [SMILES: CCCCCCCCc1ccc(CCO)cc1CCCCCCCC ; O=C(O)CCCCCCCBr ; CCCCCCCCc1ccc(OC(=O)CCCCCCCBr)cc1CCCCCCCC]

To a mixture of compound 4 (724 mg, 2.09 mmol, 1 eq.) and compound 5 (560 mg, 2.51 mmol, 1.2 eq.) in DCM (8 mL) was added EDCI (803 mg, 4.18 mmol, 2 eq.) and DMAP (102 mg, 0.84 mmol, 0.4 eq.). Then DIEA (1.078 g, 8.36 mmol, 4 eq.) was added. The reaction mixture was stirred at room temperature under nitrogen for 16 hours. TLC (petroleum ether/ethyl acetate=20/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (473 mg, 41%) as a colorless oil.

### 4. Synthesis of SW-II-134-2

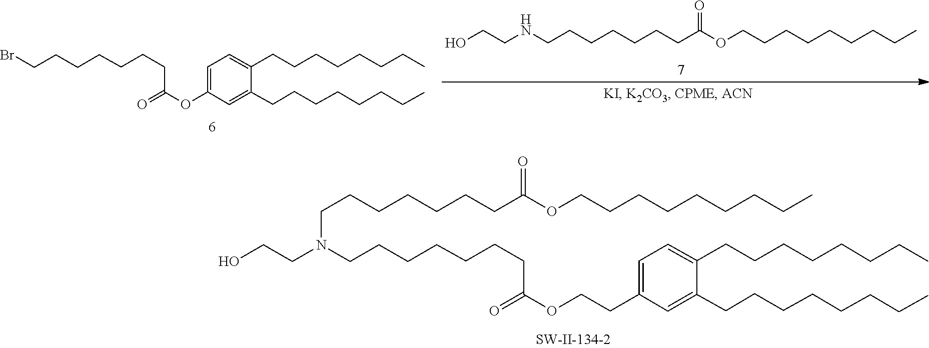

To a mixture of compound 6 (150 mg, 0.27 mmol, 1 eq.) and compound 7 (108 mg, 0.33 mmol, 1.1 eq.) in CPME/CH₃CN (2 mL/2 mL) was added potassium carbonate (225 mg, 1.63 mmol, 6 eq.) and potassium iodide (90 mg, 0.54 mmol, 2 eq.). After the addition, the mixture was stirred at 90° C. overnight under nitrogen. TLC (DCM/MeOH=15/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-134-2 (71.77 mg, 33%) as a yellow oil.

LCMS: Rt: 1.527 min; MS m/z (ELSD): 800.4 [M+H]⁺;

HPLC: 97.311% purity at ELSD; RT=9.025 min.

¹H NMR (400 MHz, CDCl₃) δ 7.06 (d, J=7.6 Hz, 1H), 6.96 (d, J=9.6 Hz, 2H), 4.25 (t, J=7.3 Hz, 2H), 4.05 (t, J=6.8 Hz, 2H), 3.80-3.66 (m, 2H), 2.86 (dd, J=12.8, 5.6 Hz, 4H), 2.78-2.67 (m, 4H), 2.60-2.52 (m, 4H), 2.29 (t, J=7.5 Hz, 4H), 1.57 (dt, J=15.8, 7.3 Hz, 14H), 1.30 (d, J=20.3 Hz, 45H), 0.88 (t, J=6.7 Hz, 9H).

¹³C NMR (101 MHz, CDCl₃) δ 173.82 (d, J=16.9 Hz), 140.73 (s), 138.82 (s), 134.91 (s), 129.71 (s), 129.23 (s), 126.19 (s), 77.36 (s), 77.14 (d, J=20.4 Hz), 76.72 (s), 65.03 (s), 64.49 (s), 57.57 (s), 56.13 (s), 54.02 (s), 34.76 (s), 34.25 (d, J=4.2 Hz), 32.76 (s), 32.37 (s), 31.89 (d, J=5.3 Hz), 31.40 (d, J=6.0 Hz), 29.84 (d, J=3.7 Hz), 29.63-29.14 (m), 28.97 (s), 28.65 (s), 26.93 (s), 25.66 (d, J=54.4 Hz), 24.80 (d, J=6.6 Hz), 22.68 (d, J=1.8 Hz), 14.12 (s).

### I. Compound SW-II-134-3

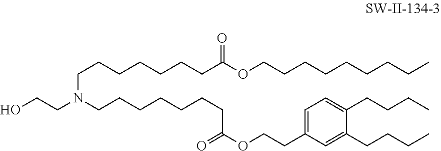 [SMILES: CCCCCCCCCOC(=O)CCCCCCCN(CCO)CCCCCCCC(=O)OCCc1ccc(CCCC)c(CCCC)c1]

### 1. Synthesis of Compound 3

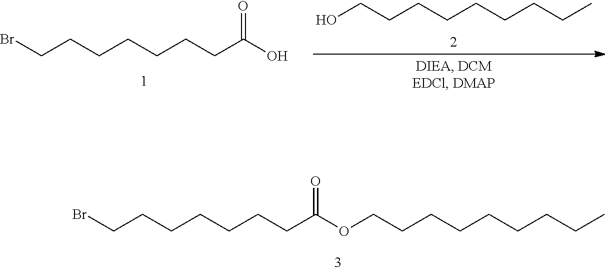

To a mixture of compound 1 (10 g, 45 mmol, 1 eq.) and compound 2 (7.8 g, 54 mmol, 1.2 eq.) in DCM (100 mL) was added EDCI (17.3 g, 90 mmol, 2 eq.) and DMAP (2.2 g, 18 mmol, 0.4 eq.). Then DIEA (23.2 g, 180 mmol, 4 eq.) was added. The reaction mixture was stirred at room temperature under nitrogen for 16 hours. TLC (petroleum ether/ethyl acetate=30/1) showed that compound 1 was consumed and the desired product was formed. The reaction mixture was extracted with ethyl acetate (20 mL) and washed with water (40 mL×3), dried over anhydrous sodium sulfate, filtered and concentrated under reduced pressure. The residue was purified by silica gel chromatography, eluting with petroleum ether/ethyl acetate (1/0-20/1), to give compound 3 (4.365 g, 28%) as a colorless oil.

### 2. Synthesis of Compound 5

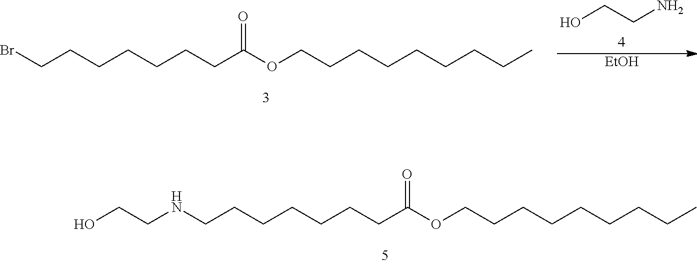

A mixture of compound 3 (5 g, 14.38 mmol, 1 eq.) and compound 4 (8.8 g, 143.7 mmol, eq.) in ethanol (2 mL) was stirred at 55° C. under nitrogen for 16 hours. TLC (DCM/MeOH=10/1) showed that new major spots were observed. The reaction mixture was extracted with ethyl acetate (50 mL) and washed with water (3×50 mL). The organic layer was dried over anhydrous sodium sulfate, filtered, and concentrated under reduced pressure. The residue was purified by silica gel chromatography, eluting with DCM/MeOH (1/0-10:1, v/v), to give compound 5 (1.008 g, 21%) as a yellow oil.

### 3. Synthesis of Compound 8

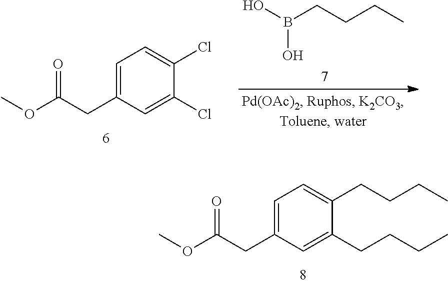

To a mixture of compound 6 (500 mg, 2.283 mmol, 1 eq.) and compound 7 (699 mg, 6.849 mmol, 3 eq) in toluene/water (5 mL/1 mL) was added palladium acetate (51 mg, 0.228 mmol, 0.1 eq.), Ruphos (213 mg, 0.457 mmol, 0.2 eq.) and potassium carbonate (945 mg, 6.849 mmol, 3 eq). The mixture was stirred at 110° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to obtain compound 8 (507 mg, 85%) as a colorless oil.

### 4. Synthesis of Compound 9

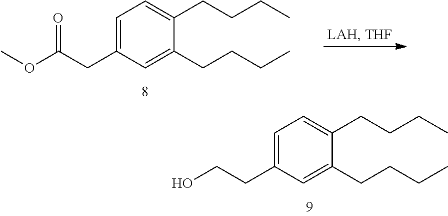 [SMILES: CCCCc1ccc(CC(=O)OC)cc1CCCC ; CCCCc1ccc(CCO)cc1CCCC]

To a mixture of compound 8 (507 mg, 1.935 mmol, 1 eq.) in THF (5 mL) was added lithium aluminum hydride (2 mL, 1.935 mmol, 1 M in THF, 1 eq.) at 0° C. under N₂. The mixture was stirred at room temperature for 3 hours. TLC (PE/EA=5/1) indicated that the reaction was completed and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 9 (492 mg, >100%) as a colorless oil, which was used without further purification.

### 5. Synthesis of Compound 10

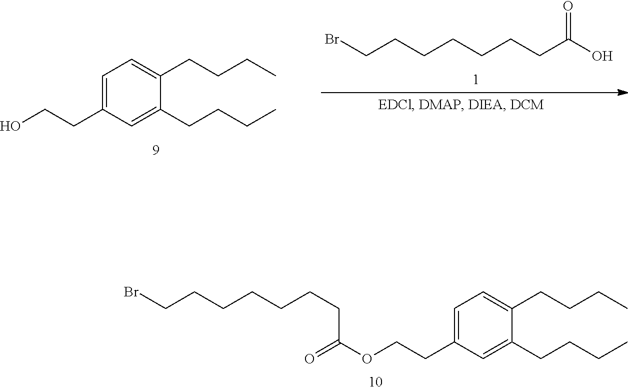 [SMILES: O=C(O)CCCCCCCBr ; CCCCc1ccc(CCO)cc1CCCC ; CCCCc1ccc(CCOC(=O)CCCCCCCBr)cc1CCCC]

To a mixture of compound 9 (492 mg, 2.103 mmol, 1 eq.) and compound 1 (563 mg, 2.523 mmol, 1.2 eq.) in DCM (5 mL) was added EDCI (808 mg, 4.206 mmol, 2 eq.) and DMAP (103 mg, 0.84 mmol, 0.4 eq.). Then DIEA (1.085 g, 8.412 mmol, 4 eq.) was added. The reaction mixture was stirred at room temperature under nitrogen for 16 hours. TLC (petroleum ether/ethyl acetate=15/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-10/1), to give compound 10 (329 mg, 36%) as a colorless oil.

### 6. Synthesis of SW-II-134-3

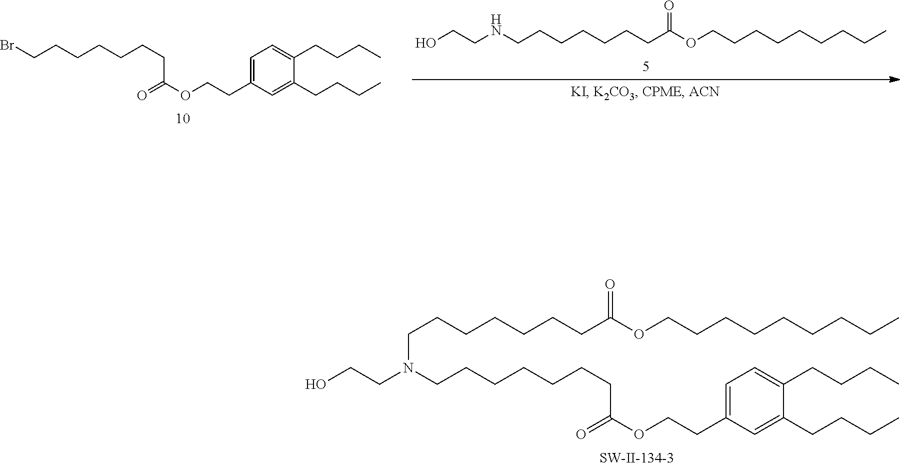 [SMILES: CCCCCCCCCOC(=O)CCCCCCCNCCO ; CCCCc1ccc(CCOC(=O)CCCCCCCBr)cc1CCCC ; CCCCCCCCCOC(=O)CCCCCCCN(CCO)CCCCCCCC(=O)OCCc1ccc(CCCC)c(CCCC)c1]

To the mixture of compound 10 (150 mg, 0.34 mmol, 1 eq.) and compound 5 (134 mg, 0.41 mmol, 1.2 eq.) in CPME/CH₃CN (2 mL/2 mL) was added potassium carbonate (282 mg, 2.04 mmol, 6 eq.) and potassium iodide (113 mg, 0.68 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-134-3 (63.59 mg, 25%) as a yellow oil.

LCMS: Rt: 1.247 min; MS m/z (ELSD): 688.3 [M+H]⁺;

HPLC: 95.945% purity at ELSD; RT=6.186 min.

¹H NMR (400 MHz, CDCl₃) δ 7.07 (d, J=7.6 Hz, 1H), 6.97 (dd, J=9.9, 2.2 Hz, 2H), 4.26 (t, J=7.2 Hz, 2H), 4.05 (t, J=6.8 Hz, 2H), 2.88 (dd, J=14.8, 7.6 Hz, 4H), 2.78-2.74 (m, 2H), 2.67-2.54 (m, 8H), 2.29 (t, J=7.5 Hz, 4H), 1.68-1.47 (m, 15H), 1.37-1.22 (m, 27H), 0.98-0.86 (m, 9H).

¹³C NMR (101 MHz, CDCl₃) δ 173.86 (d, J=17.1 Hz), 140.66 (s), 138.76 (s), 134.93 (s), 129.74 (s), 129.24 (s), 126.19 (s), 77.36 (s), 77.04 (s), 76.72 (s), 65.01 (s), 64.48 (s), 57.73 (s), 55.73 (s), 53.93 (s), 34.76 (s), 34.28 (d, J=3.9 Hz), 33.54 (d, J=4.5 Hz), 32.41 (s), 31.95 (d, J=16.5 Hz), 29.49 (s), 29.15 (dd, J=21.1, 2.4 Hz), 28.66 (s), 27.04 (s), 25.95 (d, J=3.3 Hz), 24.85 (d, J=6.6 Hz), 22.98-22.58 (m), 14.08 (d, J=7.5 Hz).

### J. Compound SW-II-135-1

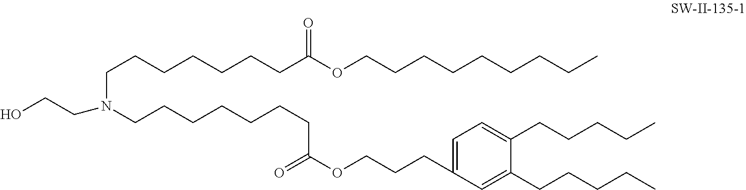 [SMILES: CCCCCCCCCOC(=O)CCCCCCCN(CCO)CCCCCCCC(=O)OCCCc1ccc(CCCCC)c(CCCCC)c1]

### 1. Synthesis of Compound 3

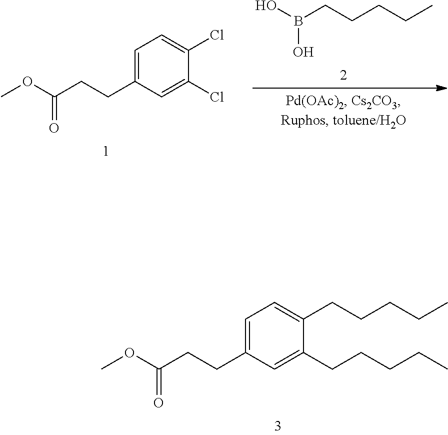

Compound 1 (500 mg, 2.16 mmol, 1.0 eq.) and Compound 2 (750 mg, 6.46 mmol, 3.0 eq.) were dissolved in Toluene/H₂O (5 mL/1 mL), and then Ruphos (201 mg, 0.43 mmol, 0.2 eq), Pd(OAc)₂ (48.5 mg, 0.22 mmol, 0.1 eq) and Cs₂CO₃ (2.10 g, 6.46 mmol, 3.0 eq.) were added therein. The reaction mixture was heated to reflux at 110° C. under nitrogen for 16 hours. TLC (petroleum ether/ethyl acetate=10/1) showed that the reaction was completed and the desired product was formed. The reaction mixture was washed with H₂O (40 mL) and extracted 3 times with EA (50 mL). The resulting organic phase was washed twice with brine (20 mL), dried over anhydrous Na₂SO₄, filtered and concentrated under reduced pressure. The residue was purified by silica gel chromatography, eluting with petroleum ether/ethyl acetate (1/0-30/1), to give compound 3 (540 mg, 82.44%) as a yellow oil.

### 2. Synthesis of Compound 4

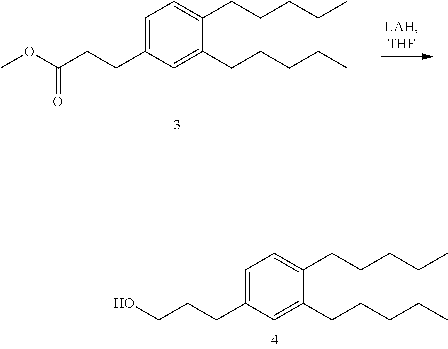 [SMILES: CCCCCc1ccc(CCC(=O)OC)cc1CCCCC ; CCCCCc1ccc(CCCO)cc1CCCCC]

LiAlH₄ (3.55 mL, 3.55 mmol, 1M in THF, 2 eq.) was added to a solution of compound 3 (540 mg, 1.78 mmol, 1.0 eq.) in THF (5 mL) at 0° C. under N₂. The reaction was allowed to warm to room temperature and stirred under nitrogen for 2 hours. TLC (PE/EtOAc=10/1) showed that the reaction was completed and new major spots were observed. The mixture was quenched with water (10 mL), then adjusted to pH=6-7 with 1M hydrochloric acid and extracted 3 times with ethyl acetate (50 mL). The organic layer was washed with brine, dried over anhydrous sodium sulfate, filtered, and concentrated under reduced pressure. The residue was purified by silica gel chromatography, eluting with PE/EA (1/0-10/1), to give compound 4 (442 mg, 90.2%) as a colorless oil.

### 3. Synthesis of Compound 6

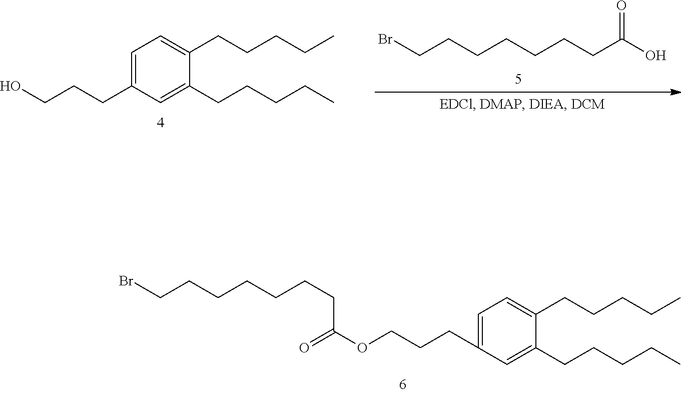 [SMILES: CCCCCc1ccc(CCCO)cc1CCCCC ; O=C(O)CCCCCCCBr ; CCCCCc1ccc(CCCOC(=O)CCCCCCCBr)cc1CCCCC]

Compound 4 (442 mg, 1.60 mmol, 1.0 eq.) and compound 5 (428.5 mg, 1.92 mmol, 1.2 eq.) were dissolved in DCM (5 mL), and then EDCI (612 mg, 3.2 mmol, 2.0 eq.) and DMAP (78.2 mg, 0.64 mmol, 0.4 eq.) were added therein. Then DIEA (826 mg, 6.4 mmol, 4.0 eq.) was added. The reaction mixture was stirred at room temperature for 16 hours under nitrogen. TLC (petroleum ether/ethyl acetate=10/1) showed that compound 4 was consumed and the desired product was formed. The reaction mixture was washed with H₂O (40 mL) and extracted 3 times with EA (50 mL). The resulting organic phase was washed twice with brine (20 mL), dried over anhydrous Na₂SO₄, filtered and concentrated under reduced pressure. The residue was purified by silica gel chromatography, eluting with petroleum ether/ethyl acetate (1/0-10/1), to give compound 3 (342 mg, 44.5%) as a yellow oil.

### 4. Synthesis of SW-II-135-1

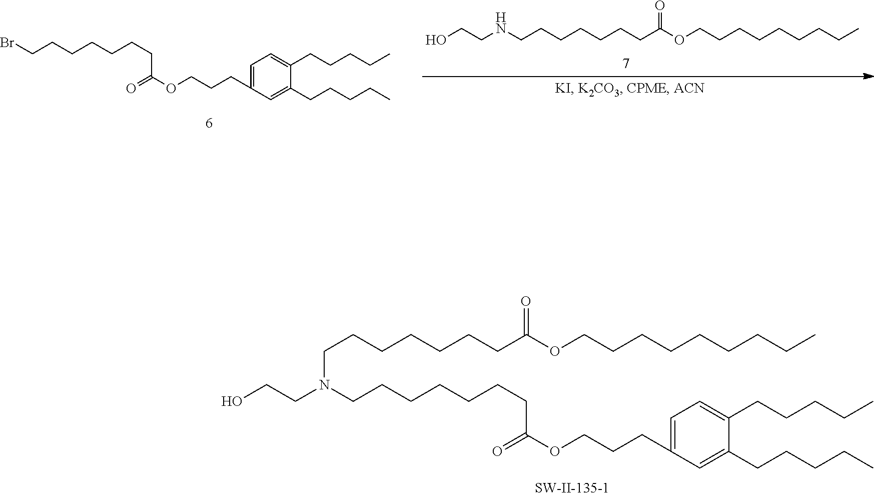 [SMILES: CCCCCCCCCOC(=O)CCCCCCCNCCO ; CCCCCc1ccc(CCCOC(=O)CCCCCCCBr)cc1CCCCC ; CCCCCCCCCOC(=O)CCCCCCCN(CCO)CCCCCCCC(=O)OCCCc1ccc(CCCCC)c(CCCCC)c1]

Compound 6 (175 mg, 0.365 mmol, 1.2 eq.) and compound 7 (100 mg, 0.304 mmol, 1.0 eq) were dissolved in CPME/CH₃CN (1 mL/1 mL), and then potassium carbonate (210 mg, 1.52 mmol, 5.0 eq) and potassium iodide (101 mg, 0.61 mmol, 2.0 eq) were added therein. After the addition, the reaction mixture was stirred at 90° C. overnight under nitrogen. TLC (DCM/MeOH=10/1) showed that the reaction was completed and the desired product was formed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to obtain compound SW-II-135-1 (83.89 mg, 55.6%) as a yellow oil.

LCMS: Rt: 1.356 min; MS m/z (ELSD): 730.5 [M+H]⁺;

HPLC: 100% purity at ELSD; RT=12.614 min.

¹H NMR (400 MHz, CDCl₃) δ 6.97 (d, J=7.6 Hz, 1H), 6.91-6.74 (m, 2H), 4.76 (s, 1H), 3.99 (dt, J=13.6, 6.4 Hz, 4H), 3.72-3.58 (m, 2H), 2.85-2.73 (m, 2H), 2.72-2.61 (m, 4H), 2.59-2.41 (m, 6H), 2.22 (dd, J=13.2, 7.2 Hz, 4H), 1.93-1.79 (m, 2H), 1.62-1.41 (m, 14H), 1.23 (d, J=24.4 Hz, 32H), 0.82 (ddd, J=13.6, 8.0, 5.6 Hz, 9H).

¹³C NMR (101 MHz, CDCl₃) δ 172.81 (d, J=6.4 Hz), 139.55 (s), 137.38 (s), 137.14 (s), 128.16 (d, J=2.4 Hz), 124.69 (s), 76.51 (s), 76.19 (s), 75.88 (s), 63.43 (s), 62.78 (s), 56.53 (s), 54.90 (s), 52.84 (s), 33.23 (d, J=2.4 Hz), 31.73 (s), 31.28 (s), 30.91 (dd, J=20.0, 6.4 Hz), 30.10 (d, J=3.2 Hz), 29.29 (s), 28.36 (d, J=22.8 Hz), 28.23 (s), 27.97 (s), 27.64 (s), 25.92 (s), 24.92 (s), 24.34 (s), 23.84 (s), 21.62 (d, J=7.6 Hz), 13.08 (d, J=4.7 Hz).

### K. Compound SW-II-135-2

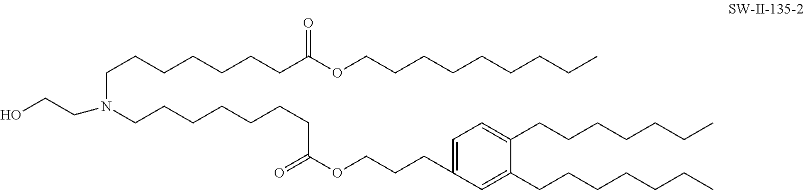 [SMILES: CCCCCCCCCOC(=O)CCCCCCCN(CCO)CCCCCCCC(=O)OCCCc1ccc(CCCCCCC)c(CCCCCCC)c1]

### 1. Synthesis of Compound 3

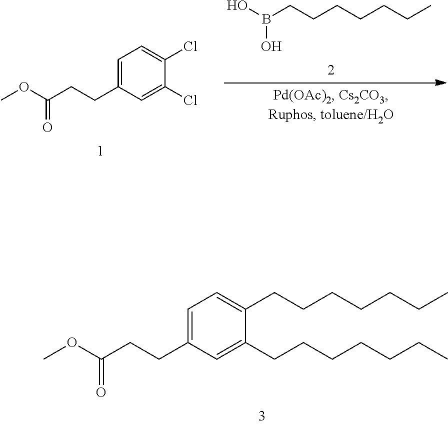

Compound 1 (500 mg, 2.16 mmol, 1.0 eq.) and Compound 2 (931 mg, 6.46 mmol, 3.0 eq.) were dissolved in Toluene/H₂O (5 mL/1 mL), and then Ruphos (201 mg, 0.43 mmol, 0.2 eq), Pd(OAc)₂ (48.5 mg, 0.22 mmol, 0.1 eq) and Cs₂CO₃ (2.10 g, 6.46 mmol, 3.0 eq.) were added therein. The reaction mixture was heated to reflux at 110° C. under nitrogen for 16 hours. TLC (petroleum ether/ethyl acetate=10/1) showed that the reaction was completed and the desired product was formed. The reaction mixture was washed with H₂O (40 mL) and extracted 3 times with EA (50 mL), the resulting organic phase was washed twice with brine (20 mL), dried over anhydrous Na₂SO₄, filtered and concentrated under reduced pressure. The residue was purified by silica gel chromatography, eluting with petroleum ether/ethyl acetate (1/0-30/1), to give compound 3 (651 mg, 84%) as a yellow oil.

### 2. Synthesis of Compound 4

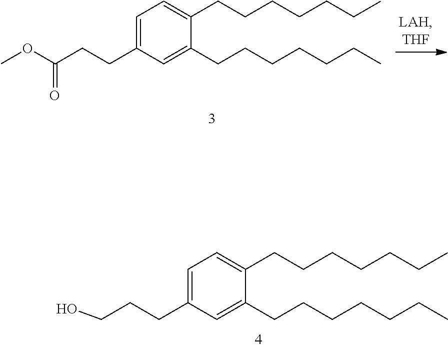 [SMILES: CCCCCCCc1ccc(CCC(=O)OC)cc1CCCCCCC ; CCCCCCCc1ccc(CCCO)cc1CCCCCCC]

LiAlH₄ (3.62 mL, 3.62 mmol, 1 M in THF, 2 eq.) was added to a solution of compound 3 (651 mg, 1.81 mmol, 1.0 eq.) in THF (7 mL) at 0° C. under N₂. The reaction was allowed to warm to room temperature and stirred under nitrogen for 2 hours. TLC (PE/EtOAc=10/1) showed that the reaction was completed and new major spots were observed. The mixture was quenched with water (10 mL), then adjusted to pH=6-7 with 1M hydrochloric acid, and extracted 3 times with ethyl acetate (50 mL). The organic layer was washed with brine, dried over anhydrous sodium sulfate, filtered, and concentrated under reduced pressure. The residue was purified by silica gel chromatography, eluting with PE/EA (1/0-10/1), to give compound 4 (571 mg, 95.2%) as a colorless oil.

### 3. Synthesis of Compound 6

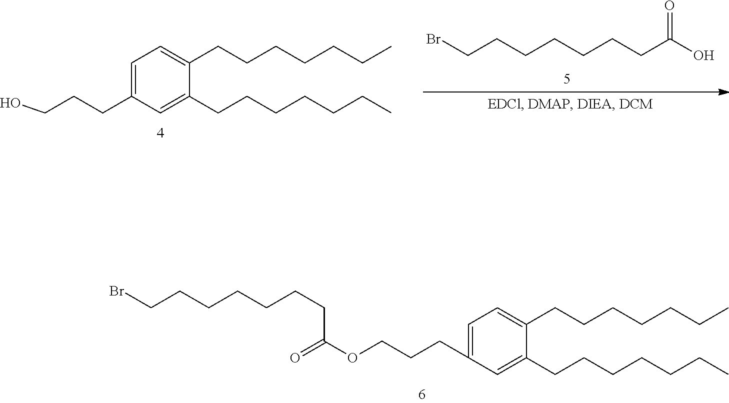 [SMILES: CCCCCCCc1ccc(CCCO)cc1CCCCCCC ; O=C(O)CCCCCCCBr ; CCCCCCCc1ccc(CCCOC(=O)CCCCCCCBr)cc1CCCCCCC]

Compound 4 (571 mg, 1.72 mmol, 1.0 eq.) and compound 5 (459 mg, 2.06 mmol, 1.2 eq.) were dissolved in DCM (6 mL), and then EDCI (657 mg, 3.44 mmol, 2.0 eq.) and DMAP (84 mg, 0.68 mmol, 0.4 eq.) were added therein. Then DIEA (887.5 mg, 6.88 mmol, 4.0 eq.) was added. The reaction mixture was stirred at room temperature for 16 hours under nitrogen. TLC (petroleum ether/ethyl acetate=10/1) showed that compound 4 was consumed and the desired product was formed. The reaction mixture was washed with H₂O (50 mL) and extracted 3 times with EA (60 mL), the resulting organic phase was washed twice with brine (25 mL), dried over anhydrous Na₂SO₄, filtered and concentrated under reduced pressure. The residue was purified by silica gel chromatography, eluting with petroleum ether/ethyl acetate (1/0-10/1), to give compound 3 (245 mg, 26.5%) as a yellow oil.

### 4. Synthesis of SW-II-135-2

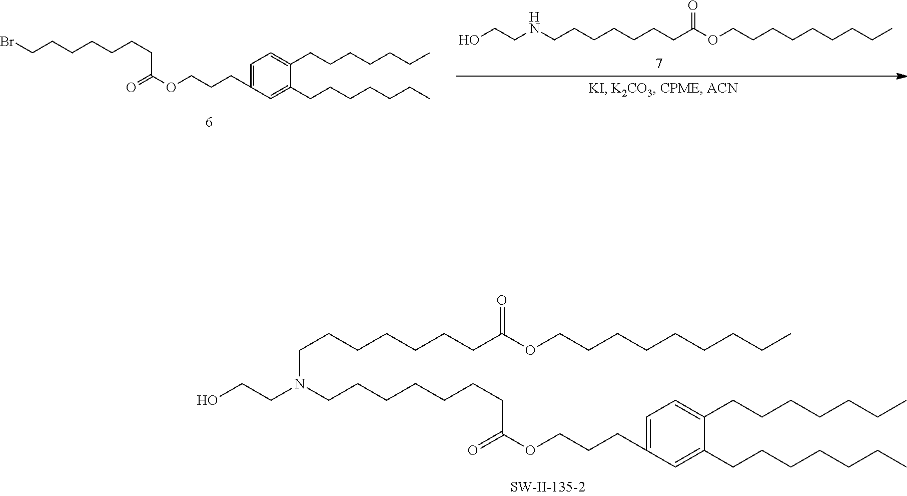 [SMILES: CCCCCCCCCOC(=O)CCCCCCCNCCO ; CCCCCCCc1ccc(CCCOC(=O)CCCCCCCBr)cc1CCCCCCC ; CCCCCCCCCOC(=O)CCCCCCCN(CCO)CCCCCCCC(=O)OCCCc1ccc(CCCCCCC)c(CCCCCCC)c1]

Compound 6 (245 mg, 0.456 mmol, 1.5 eq.) and compound 7 (100 mg, 0.3 mmol, 1.0 eq) were dissolved in CPME/CH₃CN (1 mL/1 mL), and then potassium carbonate (210 mg, 1.52 mmol, 5.0 eq) and potassium iodide (101 mg, 0.61 mmol, 2.0 eq) were added therein. After the addition, the reaction mixture was stirred at 90° C. overnight under nitrogen. TLC (DCM/MeOH=10/1) showed that the reaction was completed and the desired product was formed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to obtain compound SW-II-135-2 (31.41 mg, 21.9%) as a yellow oil.

LCMS: Rt: 1.608 min; MS m/z (ELSD): 786.4 [M+H]⁺;

HPLC: 95.16% purity at ELSD; RT=7.919 min.

¹H NMR (400 MHz, CDCl₃) δ 6.98 (d, J=7.6 Hz, 1H), 6.87 (d, J=2.4 Hz, 2H), 4.28-4.13 (m, 1H), 4.04-3.95 (m, 4H), 3.94-3.84 (m, 2H), 3.14-2.89 (m, 6H), 2.59-2.43 (m, 6H), 2.23 (dd, J=13.8, 7.2 Hz, 4H), 1.88-1.82 (m, 2H), 1.70 (s, 4H), 1.57-1.46 (m, 10H), 1.33-1.16 (m, 40H), 0.90-0.72 (m, 9H).

¹³C NMR (100 MHz, CDCl₃) δ 172.82 (d, J=6.8 Hz), 139.61 (s), 137.29 (d, J=16.4 Hz), 128.15 (s), 124.67 (s), 76.41 (s), 76.09 (s), 75.77 (s), 63.50 (s), 62.87 (s), 55.49 (s), 54.92 (s), 52.98 (s), 33.16 (d, J=2.4 Hz), 31.77 (s), 31.33 (s), 30.80 (d, J=6.5 Hz), 30.42 (d, J=3.6 Hz), 29.29 (s), 28.99-28.66 (m), 28.47 (s), 28.23 (d, J=2.8 Hz), 28.06-27.45 (m), 25.58 (s), 24.91 (s), 23.71 (s), 22.79 (s), 21.66 (s), 13.10 (s).

### L. Compound SW-II-136-2

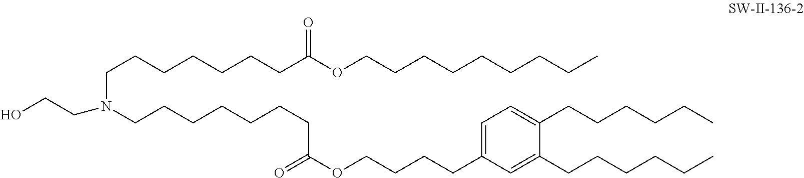 [SMILES: CCCCCCCCCOC(=O)CCCCCCCN(CCO)CCCCCCCC(=O)OCCCCc1ccc(CCCCCC)c(CCCCCC)c1]

### 1. Synthesis of Compound 3

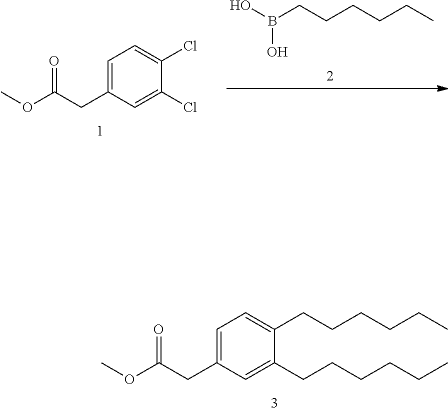

Compound 1 (3 g, 13.70 mmol, 1.0 eq.) and Compound 2 (5.34 g, 41.09 mmol, 3.0 eq.) were dissolved in Toluene/H₂O (30 mL/3 mL), and then Ruphos (1.28 g, 2.74 mmol, 0.2 eq), Pd(OAc)₂ (308.3 mg, 1.37 mmol, 0.1 eq) and K₂CO₃ (5.67 g, 41.10 mmol, 3.0 eq.) were added therein. The reaction mixture was heated to reflux at 110° C. under nitrogen for 16 hours. TLC (PE/EA=10/1) showed that the reaction was completed and the desired product was formed. The reaction mixture was washed with H₂O (90 mL) and extracted 3 times with EA (110 mL). The resulting organic phase was washed twice with brine (40 mL), dried over anhydrous Na₂SO₄, filtered and concentrated under reduced pressure. The residue was purified by silica gel chromatography, eluting with PE/EA (1/0-30/1), to give compound 3 (1.98 g, 45.5%) as a yellow oil.

### 2. Synthesis of Compound 4

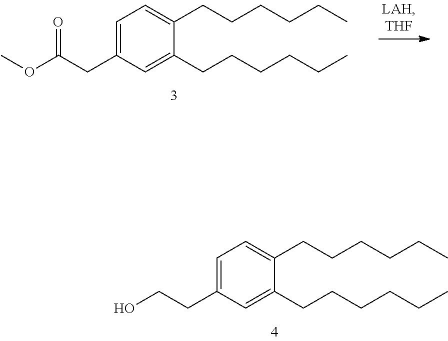 [SMILES: CCCCCCc1ccc(CC(=O)OC)cc1CCCCCC ; CCCCCCc1ccc(CCO)cc1CCCCCC]

LiAlH₄ (1 M, 12.45 mL, 2.0 eq) was added to a solution of compound 3 (1.98 g, 6.23 mmol, 1.0 eq.) in THF (20 mL) at 0° C. under N₂. The reaction was allowed to warm to room temperature and stirred under nitrogen for 2 hours. TLC (PE/EtOAc=10/1) showed that the reaction was completed and new major spots were observed. The mixture was quenched with H₂O (70 mL), then adjusted to pH 6-7 with 1 M hydrochloric acid, and extracted 3 times with EA (80 mL). The organic layer was washed with brine, dried over anhydrous Na₂SO₄, filtered, and concentrated under reduced pressure. The residue was purified by silica gel chromatography, eluting with PE/EA (1/0-10/1), to give compound 4 (1.28 g, 71.1%) as a colorless oil.

### 3. Synthesis of Compound 7

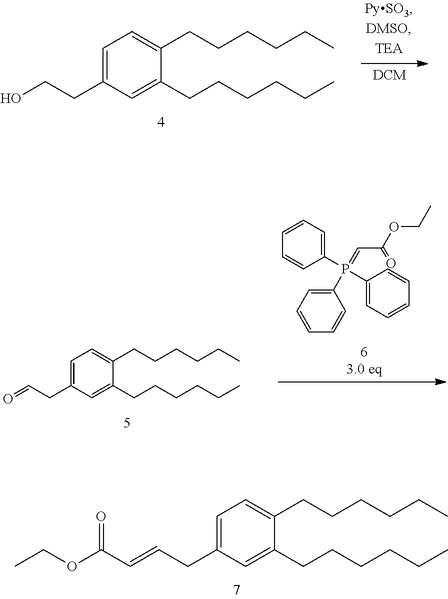

To a solution of compound 4 (900 g, 3.1 mmol, 1.0 eq.) in DCM (9 mL) were added DMSO (3.63 g, 51.72 mmol, 15 eq), TEA (1.25 g, 12.4 mmol, 4.0 eq) and PySO₃ (1.27 g, 7.97 mmol, 2.57 eq) at 0° C. under N₂. The mixture was stirred at 0° C. for 30 minutes, then allowed to warm to room temperature and stirred for 90 minutes under nitrogen. Then compound 6 (4.74 g, 13.62 mmol, 3.0 eq.) was added to the mixture, and the reaction mixture was reacted at 25° C. for 2 hours under nitrogen. TLC (PE/EA=10/1) showed that the reaction was completed and the desired product was formed. The reaction mixture was washed with H₂O (60 mL) and extracted 3 times with EA (70 mL). The resulting organic phase was washed twice with brine (40 mL), dried over anhydrous Na₂SO₄, filtered and concentrated under reduced pressure. The residue was purified by silica gel chromatography, eluting with PE/EA (1/0-10/1), to give compound 7 (345 mg, 27.9%) as a yellow oil.

### 4. Synthesis of Compound 8

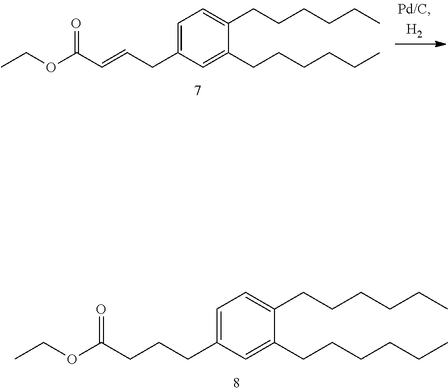

Compound 7 (340 mg, 0.95 mmol, 1.0 eq.) and Pd/C (100 mg) were added to MeOH (4 ml), and the reaction mixture was stirred at room temperature under hydrogen for 16 h. TLC (PE/EA=10/1) showed that the starting material was consumed and the desired product was produced. The reaction mixture was filtered through Celite and washed with MeOH (40 mL×2), dried over anhydrous Na₂SO₄ and the filtrate was concentrated under reduced pressure to obtain compound 8 (298 mg, 88.2%) as a pale yellow oil.

### 5. Synthesis of Compound 9

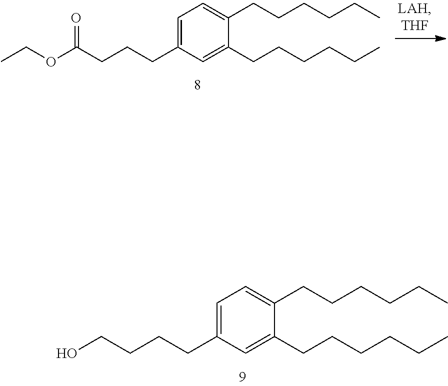 [SMILES: CCCCCCc1ccc(CCCC(=O)OCC)cc1CCCCCC ; CCCCCCc1ccc(CCCCO)cc1CCCCCC]

LiAlH₄ (1 M, 1.66 mL, 2.0 eq) was added to a solution of compound 8 (298 mg, 0.83 mmol, 1.0 eq.) in THF (3 mL) at 0° C. under N₂. The reaction was allowed to warm to room temperature and stirred under nitrogen for 2 hours. TLC (PE/EtOAc=10/1) showed that the reaction was completed and new major spots were observed. The mixture was quenched with H₂O (20 mL), then adjusted to pH 6-7 with 1 M hydrochloric acid and extracted 3 times with EA (30 mL). The organic layer was washed with brine, dried over anhydrous Na₂SO₄, filtered, and concentrated under reduced pressure. The residue was purified by silica gel chromatography, eluting with PE/EA (1/0-10/1), to give compound 9 (254 mg, 98.3%) as a colorless oil.

### 6. Synthesis of Compound 11

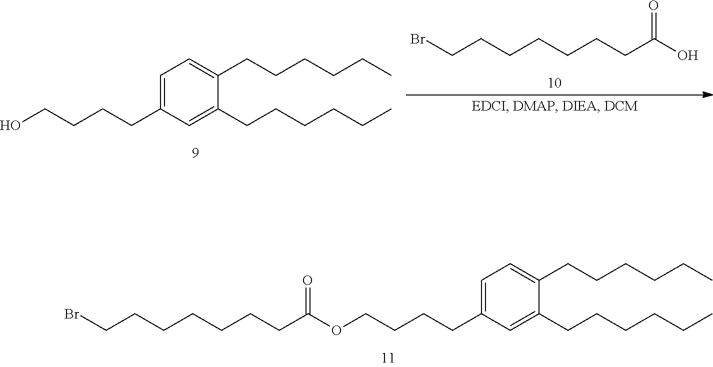 [SMILES: O=C(O)CCCCCCCBr ; CCCCCCc1ccc(CCCCO)cc1CCCCCC ; CCCCCCc1ccc(CCCCOC(=O)CCCCCCCBr)cc1CCCCCC]

Compound 9 (254 mg, 0.80 mmol, 1.0 eq.) and compound 10 (214 mg, 0.96 mmol, 1.2 eq.) were dissolved in DCM (3 mL). EDCI (305.6 mg, 1.6 mmol, 2.0 eq.) and DMAP (39 mg, 0.32 mmol, 0.4 eq.) were added therein. Then DIEA (412.8 mg, 3.2 mmol, 4.0 eq.) was added. The reaction mixture was stirred at room temperature under nitrogen for 16 hours. TLC (PE/EA=10/1) showed that compound 9 was consumed and the desired product was formed. The reaction mixture was adjusted to pH=4-6 with 1 M hydrochloric acid, and extracted 3 times with EA (30 mL). The resulting organic phase was washed twice with brine (15 mL), dried over anhydrous Na₂SO₄, filtered and concentrated under reduced pressure. The residue was purified by silica gel chromatography, eluting with PE/EA (1/0-7/1), to give compound 11 (210 mg, 50.5%) as a yellow oil.

### 7. Synthesis of SW-II-136-2

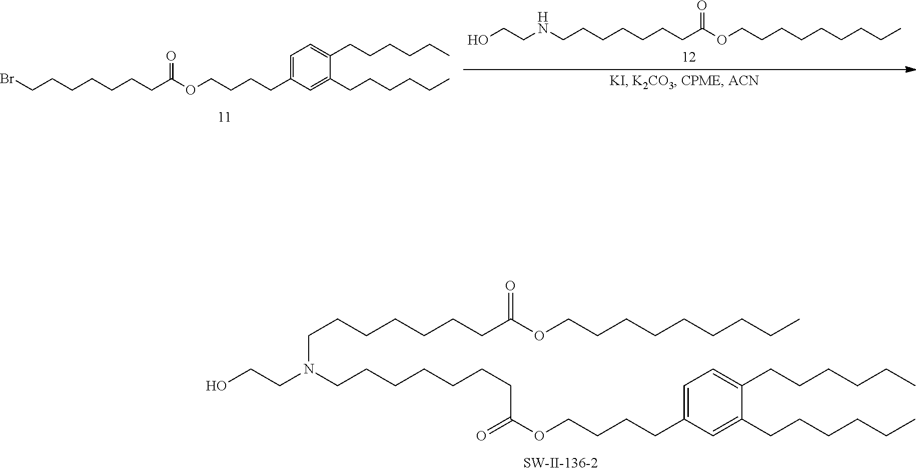

Compound 11 (200 mg, 0.38 mmol, 1.2 eq.) and compound 12 (105 mg, 0.32 mmol, 1.0 eq) were dissolved in CPME/CH₃CN (1.5 mL/1.5 mL), and then K₂CO₃ (220.2 mg, 1.60 mmol, 5.0 eq) and KI (106 mg, 0.64 mmol, 2.0 eq) were added therein. After the addition, the reaction mixture was stirred at 90° C. overnight under nitrogen. TLC (DCM/MeOH=10/1) showed that the reaction was completed and the desired product was formed. The mixture was extracted with EA and washed with water. The organic layer was dried over anhydrous Na₂SO₄, filtered, and concentrated under reduced pressure. The residue was purified by silica gel chromatography, eluting with DCM/MeOH (1/0-10:1, v/v), to give compound SW-II-136-2 (208 mg, 90.4%) as a yellow oil.

LCMS: Rt: 2.146 min; MS m/z (ELSD): 773.3 [M+H]⁺;

HPLC: 99.49% purity at ELSD; RT=8.055 min.

¹H NMR (400 MHz, CDCl₃) δ 7.04 (d, J=7.6 Hz, 1H), 6.92 (d, J=9.6 Hz, 2H), 4.45 (s, 1H), 4.06 (dd, J=12.0, 5.2 Hz, 4H), 3.64 (t, J=5.2 Hz, 2H), 2.72 (t, J=5.2 Hz, 2H), 2.65-2.50 (m, 10H), 2.29 (t, J=7.6 Hz, 4H), 1.69-1.48 (m, 18H), 1.41-1.24 (m, 36H), 0.95-0.78 (m, 9H).

¹³C NMR (101 MHz, CDCl₃) δ 173.86 (d, J=2.8 Hz), 140.48 (s), 139.24 (s), 138.01 (s), 129.13 (d, J=14.8 Hz), 125.67 (s), 77.37 (s), 77.05 (s), 76.73 (s), 64.45 (s), 64.23 (s), 57.88 (s), 55.91 (s), 53.94 (s), 35.07 (s), 34.29 (d, J=3.2 Hz), 32.79 (s), 32.35 (s), 31.82 (d, J=8.4 Hz), 31.38 (s), 29.50 (d, J=2.4 Hz), 29.16 (dd, J=18.0, 2.0 Hz), 28.66 (s), 28.35 (s), 27.78 (s), 27.08 (s), 26.02 (d, J=17.2 Hz), 24.89 (d, J=1.6 Hz), 22.65 (s), 14.10 (s).

### M. Compound SW-II-137-1

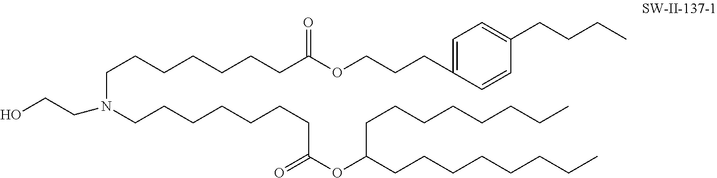 [SMILES: CCCCCCCCC(CCCCCCCC)OC(=O)CCCCCCCN(CCO)CCCCCCCC(=O)OCCCc1ccc(CCCC)cc1]

### 1. Synthesis of Compound 3

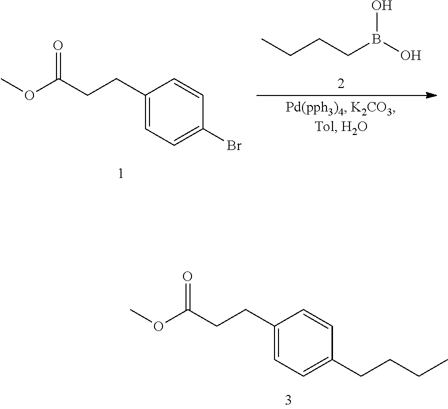

Compound 1 (500 mg, 1.95 mmol, 1.0 eq.) was dissolved in toluene (5.0 mL), then compound 2 (239 mg, 2.34 mmol, 1.2 eq.), Pd(PPh₃)₄ (225 mg, 0.19 mmol, 0.1 eq.), water (1 mL) and K₂CO₃ (808 g, 5.85 mmol, 3.0 eq.) were added. The reaction was carried out at 110° C. for 3 hours under nitrogen. TLC (PE/EA=5/1) showed that the starting material was consumed and the desired product was formed. The reaction was quenched with H₂O (70 mL) and extracted with EA (80 mL×3). The organic phases were combined and washed with saturated brine (2×30 mL), dried over anhydrous Na₂SO₄, filtered and spin-dried under reduced pressure. The residue was purified by silica gel chromatography, eluting with PE/EA (1/0-5:1, v/v), to obtain a colorless oily compound (320 mg, 70%).

### 2. Synthesis of Compound 4

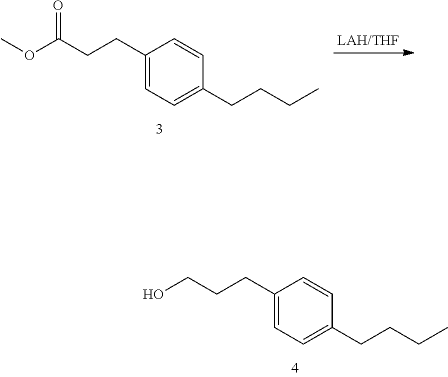 [SMILES: CCCCc1ccc(CCC(=O)OC)cc1 ; CCCCc1ccc(CCCO)cc1]

Compound 3 (300 mg, 1.28 mmol, 1.0 eq.) was dissolved in THF (4.0 mL), and LAH (97 mg, 2.56 mmol, 2.0 eq) was added at 0° C. under nitrogen. Then the reaction was carried out at room temperature for 2 hours. TLC (PE/EA=10/1) showed that the reaction of the starting material was completed and the desired product was produced. The reaction was quenched by adding HCl (1M, 4 mL) solution and H₂O (10 mL), and extracted with EA (50 mL×3). The organic phase was washed with saturated brine (2×30 mL), dried over anhydrous Na₂SO₄, filtered and spin-dried under reduced pressure. The residue was purified by silica gel chromatography, eluting with PE/EA (1/0-10/1, v/v), to obtain compound 4 (224 mg, 84.8%) as a yellow oil.

### 3. Synthesis of Compound 6

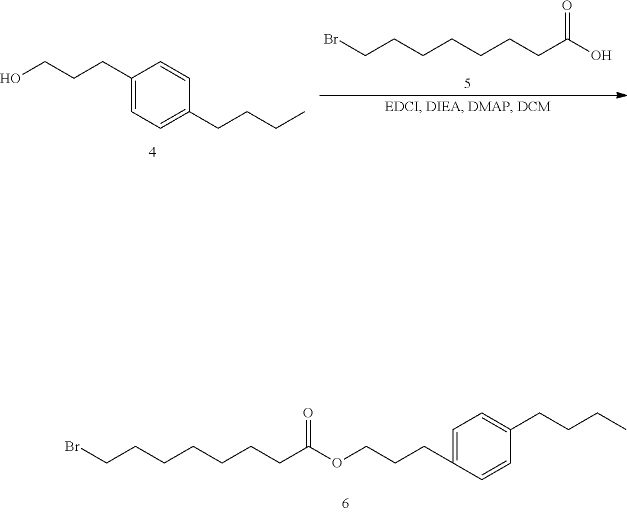 [SMILES: O=C(O)CCCCCCCBr ; CCCCc1ccc(CCCO)cc1 ; CCCCc1ccc(CCCOC(=O)CCCCCCCBr)cc1]

Compound 4 (90 mg, 0.47 mmol, 1.0 eq.) was dissolved in DCM (3.0 mL), and then compound 5 (127 mg, 0.56 mmol, 1.2 eq.), EDCI (180 mg, 0.94 mmol, 2.0 eq.), DIEA (242 mg, 1.88 mmol, 4.0 eq.) and DMAP (23 mg, 0.18 mmol, 0.4 eq.) were added. Then, the reaction was carried out overnight at room temperature under nitrogen. TLC (PE/EA=20/1) showed that the reaction of the starting material was completed and the desired product was formed. The reaction was quenched with HCl (1M) solution, adjusted to pH=4-6, and extracted with EA (40 mL×3). The organic phases were combined and washed with saturated brine (2×30 mL), dried over anhydrous Na₂SO₄, filtered and spin-dried under reduced pressure. The residue was purified by silica gel chromatography, eluting with PE/EA (1/0-20/1, v/v), to obtain compound 6 (90 mg, 48.6%) as a colorless oil.

### 4. Synthesis of SW-II-137-1

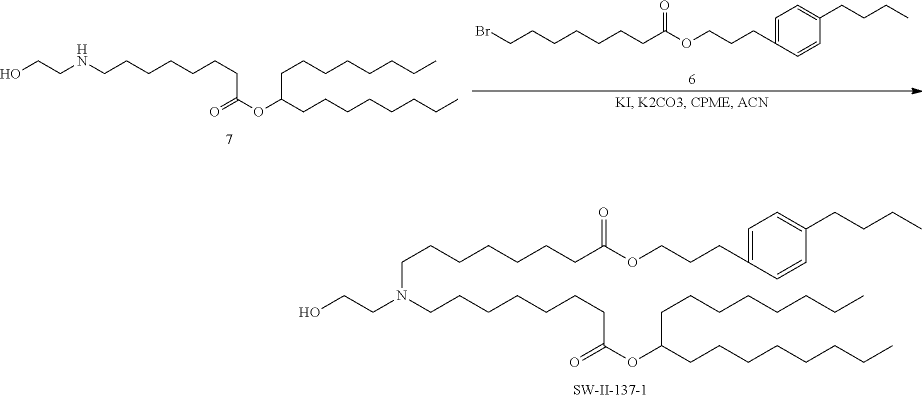 [SMILES: CCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCC)cc1]

Compound 6 (90 mg, 0.25 mmol, 1.0 eq.) was dissolved in MeCN (2 mL), and then compound 7 (110 mg, 0.25 mmol, 1.0 eq), KI (76 mg, 0.50 mmol, 2.0 eq), CPME (2 mL), and K₂CO₃ (157 mg, 1.25 mmol, 5.0 eq) were added. The reaction was carried out overnight at 90° C. under nitrogen. TLC (DCM/MeOH=10/1) showed that the reaction of the starting material was completed and the desired product was formed. The reaction was quenched with water (50 mL), and extracted with EA (40 mL×3). The organic phases were combined and washed with saturated brine (2×30 mL), dried over anhydrous Na₂SO₄, filtered and spin-dried under reduced pressure. The residue was purified by silica gel chromatography, eluting with DCM/MeOH (1/0-10/1, v/v), to obtain the compound (98 mg, 52.12%, SW-II-137-1) as a yellow oil.

LCMS: Rt: 1.596 min; MS m/z (ELSD): 758.4 [M+H]⁺;

HPLC: 98.02% purity at ELSD; RT=5.993 min.

¹H NMR (400 MHz, CDCl₃) δ 7.02 (d, J=8.8 Hz, 4H), 4.92-4.71 (m, 1H), 4.01 (t, J=6.4 Hz, 2H), 3.78 (s, 1H), 3.55 (t, J=5.2 Hz, 2H), 2.76-2.40 (m, 10H), 2.21 (dd, J=15.6, 7.7 Hz, 4H), 1.95-1.80 (m, 2H), 1.49 (ddd, J=24.4, 15.8, 6.2 Hz, 15H), 1.34-1.13 (m, 37H), 0.82 (dt, J=13.6, 7.2 Hz, 9H).

¹³C NMR (101 MHz, CDCl₃) δ 173.79 (s), 173.57 (s), 140.49 (s), 138.30 (s), 128.43 (s), 128.22 (s), 77.43 (s), 77.11 (s), 76.79 (s), 74.11 (s), 63.66 (s), 57.96 (s), 55.75 (s), 53.90 (s), 35.22 (s), 34.63 (s), 34.20 (d, J=11.6 Hz), 33.70 (s), 31.80 (d, J=11.2 Hz), 30.30 (s), 29.51 (d, J=2.8 Hz), 29.13 (dd, J=9.6, 6.8 Hz), 27.12 (d, J=2.8 Hz), 26.29 (s), 25.31 (s), 24.97 (d, J=15.6 Hz), 22.66 (s), 22.37 (s), 14.02 (d, J=15.2 Hz).

### N. Compound SW-II-137-2

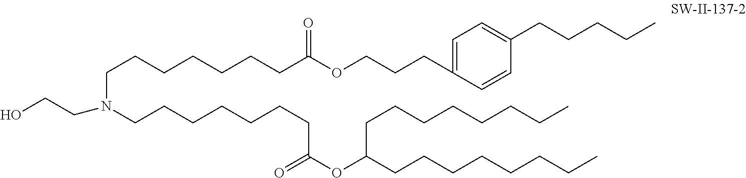 [SMILES: CCCCCCCCC(CCCCCCCC)OC(=O)CCCCCCCN(CCO)CCCCCCCC(=O)OCCCc1ccc(CCCCC)cc1]

### 1. Synthesis of Compound 3

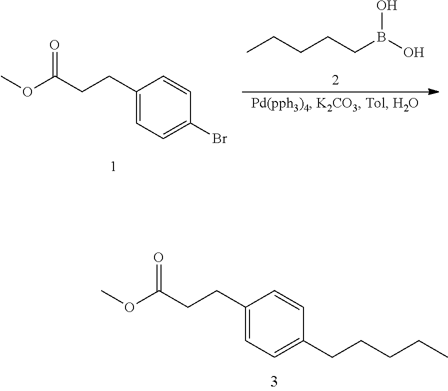

Compound 1 (500 mg, 2.06 mmol, 1.0 eq.), Compound 2 (286 mg, 2.47 mmol, 1.2 eq.), Pd(PPh₃)₄ (119 mg, 0.1 mmol, 0.1 eq) and K₂CO₃ (851 mg, 6.21 mmol, 3.0 eq.) were dissolved in toluene (5.0 mL), and then water (0.5 mL) was added therein. Then, the reaction was carried out at 110° C. for 3 hours under nitrogen. TLC (PE/EA=5/1) showed that the reaction of the starting material was completed and the desired compound was formed. The reaction was quenched with H₂O (70 mL), and extracted with EA (80 mL×3). The organic phase was washed with saturated brine (2×30 mL), dried over anhydrous Na₂SO₄, filtered and spin-dried under reduced pressure. The residue was purified by silica gel chromatography, eluting with (PE/EA=5/1, v/v), to obtain compound 3 (420 mg, 87.5%) as a colorless oil.

### 2. Synthesis of Compound 4

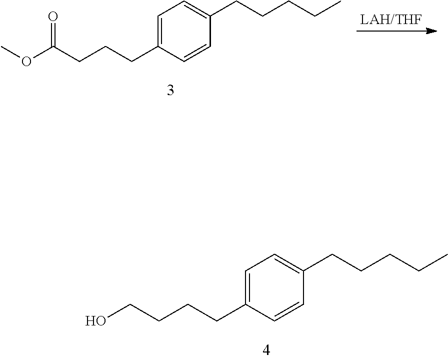 [SMILES: CCCCCc1ccc(CCCC(=O)OC)cc1 ; CCCCCc1ccc(CCCCO)cc1]

Compound 3 (420 mg, 1.78 mmol, 1.0 eq.) was dissolved in THF (3.0 mL), and LAH (1 M, 7 mL, 2.0 eq) was added dropwise at 0° C. under nitrogen. Then, the reaction was carried out at room temperature for 2 hours. TLC (PE/EA=5/1) showed that the reaction of the starting material was completed and the desired product was formed. The reaction was quenched with HCl (1M, 4 mL) solution and H₂O (10 mL), and extracted with EA (50 mL×3). The organic phase was washed with saturated brine (2×30 mL), dried over anhydrous Na₂SO₄, filtered and spin-dried under reduced pressure. The residue was purified by silica gel chromatography, eluting with (PE/EA=5/1, v/v), to obtain compound 4 (320 mg, 94%) as a colorless oil.

### 3. Synthesis of Compound 6

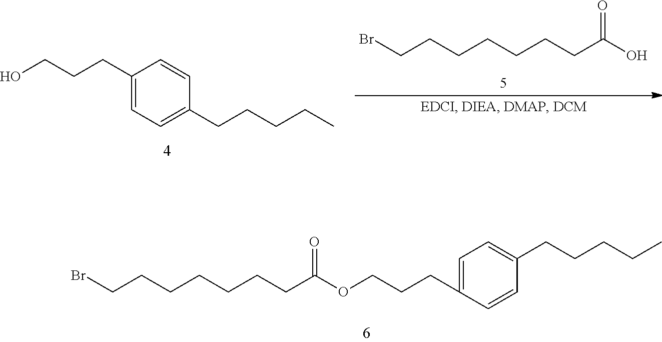 [SMILES: O=C(O)CCCCCCCBr ; CCCCCc1ccc(CCCO)cc1 ; CCCCCc1ccc(CCCOC(=O)CCCCCCCBr)cc1]

Compound 4 (320 mg, 1.55 mmol, 1.0 eq.) was dissolved in DCM (4.0 mL), and then compound 5 (416 mg, 1.86 mmol, 1.2 eq.), EDCI (594 mg, 3.11 mmol, 2.0 eq.), DIEA (802 mg, 6.21 mmol, 4.0 eq.) and DMAP (76 mg, 0.62 mmol, 0.4 eq.) were added therein. Then, the reaction was carried out overnight at room temperature under nitrogen. TLC (PE/EA=20/1) showed that the reaction of the starting material was completed and the desired product was formed. The reaction was quenched with HCl (1M) solution and adjusted to pH=4-6, and extracted with DCM (60 mL×3). The organic phase was washed with saturated brine (2×35 mL), dried over anhydrous Na₂SO₄, filtered and spin-dried under reduced pressure. The residue was purified by silica gel chromatography, eluting with (PE/EA=5/1, v/v), to obtain compound 6 (300 mg, 47.17%) as a colorless oil.

### 4. Synthesis of SW-II-137-2

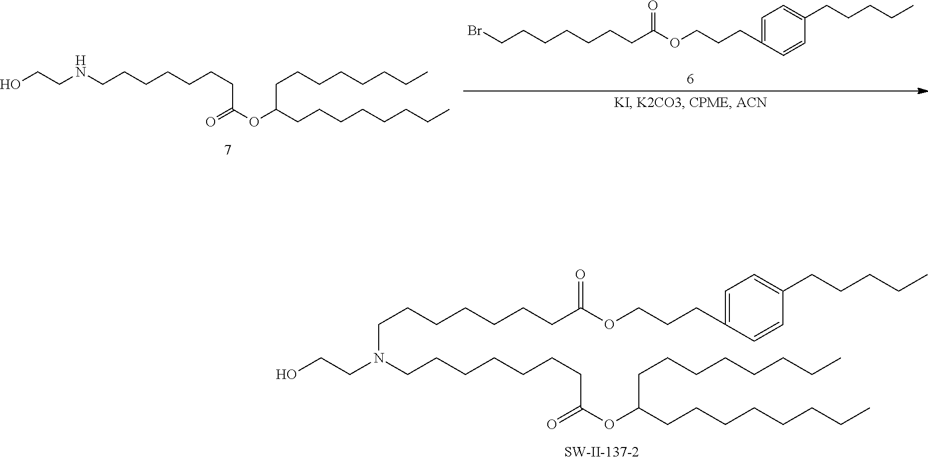 [SMILES: CCCCCc1ccc(CCCOC(=O)CCCCCCCBr)cc1 ; CCCCCCCCC(CCCCCCCC)OC(=O)CCCCCCCNCCO ; CCCCCCCCC(CCCCCCCC)OC(=O)CCCCCCCN(CCO)CCCCCCCC(=O)OCCCc1ccc(CCCCC)cc1]

Compound 6 (167 mg, 0.41 mmol, 1.2 eq.), compound 7 (150 mg, 0.34 mmol, 1.0 eq), KI (113 mg, 0.68 mmol, 2.0 eq) and CPME (2 mL) were dissolved in MeCN (2 mL), and then K₂CO₃ (235 mg, 1.70 mmol, 5.0 eq) was added. The reaction was carried out overnight at 90° C. under nitrogen. TLC (DCM/MeOH=10/1) showed that the reaction of the starting material was completed and the desired product was produced. The reaction was quenched with water (50 mL), and extracted with EA (60 ml×3). The organic phase was dried over anhydrous Na₂SO₄, filtered and spin-dried under reduced pressure. The residue was purified by silica gel chromatography, eluting with DCM/MeOH (1/0-10/1, v/v), to obtain the compound (105 mg, 40.3%, SW-II-137-2) as a pale yellow oil.

LCMS: Rt: 1.660 min; MS m/z (ELSD): 772.4 [M+H]⁺;

HPLC: 98.38% purity at ELSD; RT=8.743 min.

¹H NMR (400 MHz, CDCl₃) δ 7.10 (d, J=8.8 Hz, 4H), 5.04-4.74 (m, 1H), 4.08 (t, J=6.4 Hz, 2H), 3.58 (t, J=5.2 Hz, 2H), 2.65 (dd, J=9.6, 5.6 Hz, 4H), 2.60-2.44 (m, 6H), 2.29 (dd, J=16.4, 7.6 Hz, 4H), 2.01-1.88 (m, 2H), 1.59 (dt, J=9.2, 7.2 Hz, 6H), 1.54-1.42 (m, 8H), 1.39-1.11 (m, 41H), 0.88 (dt, J=11.8, 6.0 Hz, 9H).

¹³C NMR (101 MHz, CDCl₃) δ 173.86 (s), 173.63 (s), 140.59 (s), 138.34 (s), 128.45 (s), 128.24 (s), 77.36 (s), 77.04 (s), 76.72 (s), 74.14 (s), 63.69 (s), 58.11 (s), 55.71 (s), 53.90 (s), 35.53 (s), 34.68 (s), 34.23 (d, J=14.8 Hz), 31.82 (d, J=11.6 Hz), 31.56 (s), 31.26 (s), 30.32 (s), 29.53 (d, J=2.8 Hz), 29.19 (dd, J=8.0, 4.4 Hz), 27.20 (d, J=2.4 Hz), 26.64 (s), 25.33 (s), 25.02 (d, J=15.6 Hz), 22.62 (d, J=11.6 Hz), 14.08 (d, J=8.0 Hz).

### O. Compound SW-II-137-3

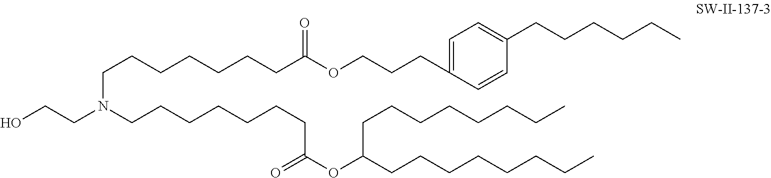 [SMILES: CCCCCCCCC(CCCCCCCC)OC(=O)CCCCCCCN(CCO)CCCCCCCC(=O)OCCCc1ccc(CCCCCC)cc1]

### 1. Synthesis of Compound 3

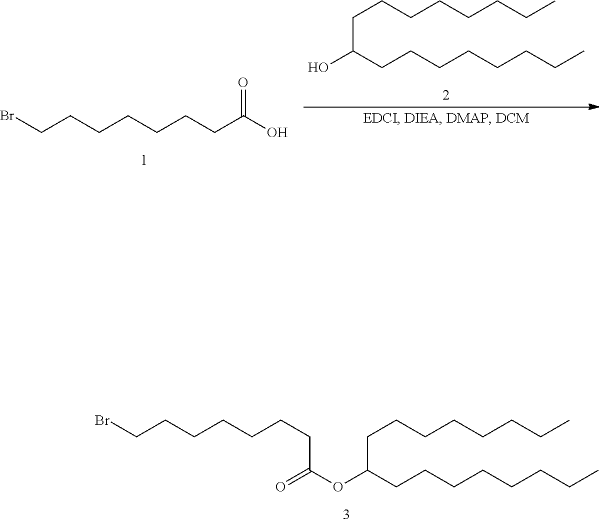 [SMILES: CCCCCCCCC(O)CCCCCCCC ; O=C(O)CCCCCCCBr ; CCCCCCCCC(CCCCCCCC)OC(=O)CCCCCCCBr]

To a mixture of compound 1 (11.8 g, 53 mmol, 1.2 eq.) and compound 2 (11.2 g, 44 mmol, 1 eq.) in DCM (110 mL) was added EDCI (16.9 g, 88 mmol, 2 eq.) and DMAP (2.1 g, 18 mmol, 0.4 eq.). Then, DIEA (22.7 g, 176 mmol, 4 eq.) was added. The reaction mixture was stirred at room temperature under nitrogen for 16 hours. TLC (petroleum ether/ethyl acetate=30/1) showed that compound 1 was consumed and the desired product was formed. The reaction mixture was extracted with ethyl acetate (200 mL) and washed with water (200 mL×3), dried over anhydrous sodium sulfate, filtered and concentrated under reduced pressure. The residue was purified by silica gel chromatography, eluting with petroleum ether/ethyl acetate (1/0-20/1), to give compound 3 (7.391 g, 37%) as a colorless oil.

### 2. Synthesis of Compound 5

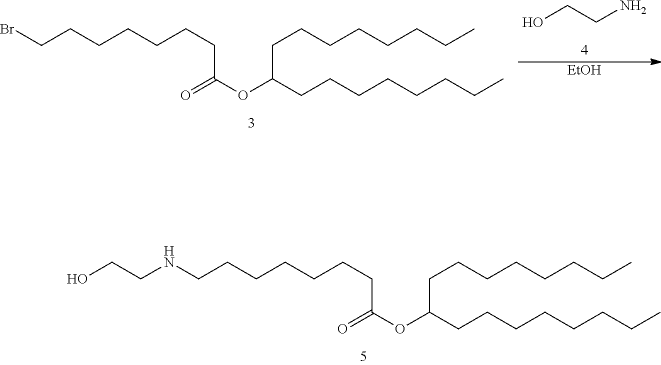

A mixture of compound 3 (7.391 mg, 16.07 mmol, 1 eq.) and compound 4 (29.4 g, 482.02 mmol, 30 eq.) in ethanol (2 mL) was stirred at 55° C. under nitrogen for 16 hours. TLC (DCM/MeOH=10/1) showed that new major spots were observed. The reaction mixture was extracted with ethyl acetate (100 mL) and washed with water (3×100 mL). The organic layer was dried over anhydrous sodium sulfate, filtered, and concentrated under reduced pressure. The residue was purified by silica gel chromatography, eluting with DCM/MeOH (1/0-10:1, v/v), to give compound 5 (3.695 g, 52%) as a yellow oil.

### 3. Synthesis of Compound 8

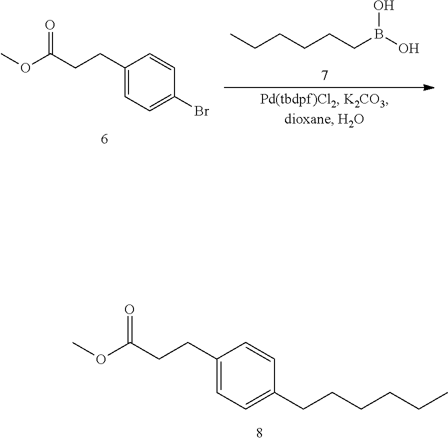

To the mixture of compound 6 (1 g, 4.12 mmol, 1 eq.) and compound 7 (803 g, 6.17 mmol, 1.5 eq) in 1,4-dioxane/water (10 mL/1 mL) was added Pd(dtbpf)Cl₂ (269 mg, 0.41 mmol, 0.1 eq.) and potassium carbonate (1.7 g, 12.36 mmol, 3 eq). The mixture was stirred at 100° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to obtain compound 8 (568 mg, 56%) as a colorless oil.

### 4. Synthesis of Compound 9

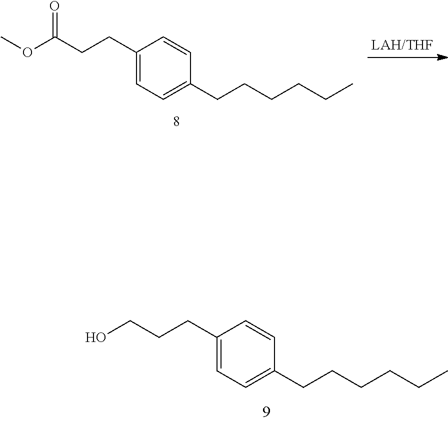 [SMILES: CCCCCCc1ccc(CCC(=O)OC)cc1 ; CCCCCCc1ccc(CCCO)cc1]

To a mixture of compound 8 (568 mg, 2.29 mmol, 1 eq.) in THF (6 mL) was added lithium aluminum hydride (2.3 mL, 2.29 mmol, 1 M in THF, 1 eq.) at 0° C. under nitrogen. The mixture was stirred at room temperature for 3 hours. TLC (PE/EA=5/1) indicated that the reaction was completed and new major spots were observed. The mixture was quenched with water (2.3 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 9 (541 mg, >100%) as a colorless oil, which was used without further purification.

### 5. Synthesis of Compound 10

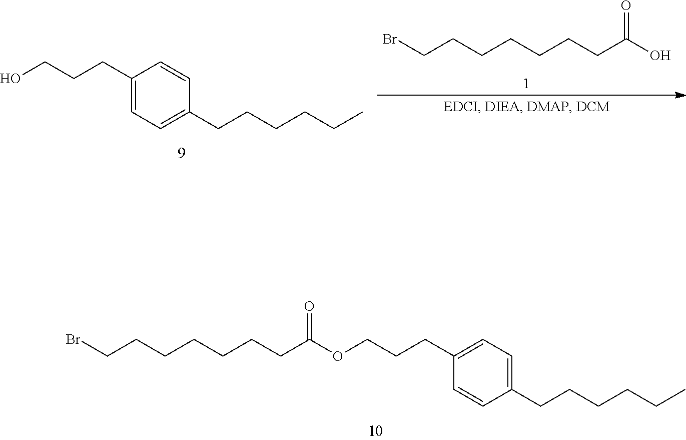 [SMILES: O=C(O)CCCCCCCBr ; CCCCCCc1ccc(CCCO)cc1 ; CCCCCCc1ccc(CCCOC(=O)CCCCCCCBr)cc1]

To a mixture of compound 9 (441 mg, 2 mmol, 1 eq.) and compound 1 (536 mg, 2.4 mmol, 1.2 eq.) in DCM (5 mL) was added EDCI (768 mg, 4 mmol, 2 eq.) and DMAP (98 mg, 0.8 mmol, 0.4 eq.). Then DIEA (1.032 g, 8 mmol, 4 eq.) was added. The reaction mixture was stirred at room temperature under nitrogen for 16 hours. TLC (petroleum ether/ethyl acetate=10/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-10/1), to give compound 10 (372 mg, 44%) as a colorless oil.

### 6. Synthesis of SW-II-137-3

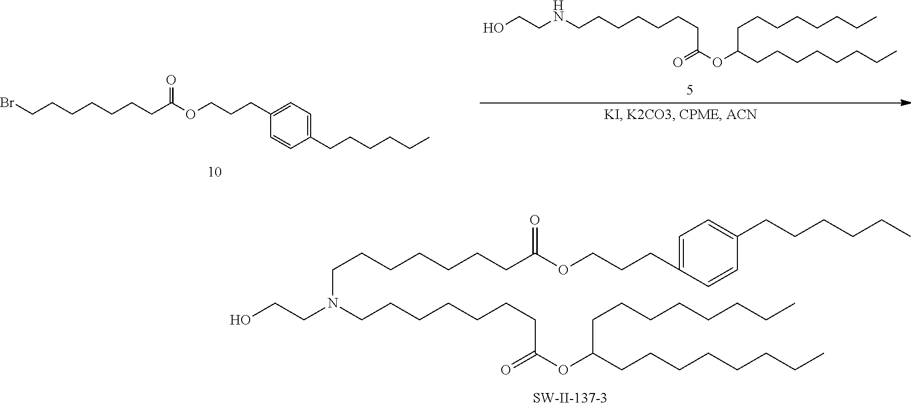 [SMILES: CCCCCCCCC(CCCCCCCC)OC(=O)CCCCCCCNCCO ; CCCCCCc1ccc(CCCOC(=O)CCCCCCCBr)cc1 ; CCCCCCCCC(CCCCCCCC)OC(=O)CCCCCCCN(CCO)CCCCCCCC(=O)OCCCc1ccc(CCCCCC)cc1]

To a mixture of compound 10 (150 mg, 0.353 mmol, 1 eq.) and compound 5 (156 mg, 0.353 mmol, 1 eq.) in CPME/CH₃CN (2 mL/2 mL) was added potassium carbonate (244 mg, 1.765 mmol, 6 eq.) and potassium iodide (117 mg, 0.706 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-137-3 (56.17 mg, 20%) as a yellow oil.

LCMS: Rt: 1.550 min; MS m/z (ELSD): 786.4 [M+H]⁺;

HPLC: 98.597% purity at ELSD; RT=13.153 min.

¹H NMR (400 MHz, CDCl₃) δ 7.09 (s, 4H), 4.92-4.78 (m, 1H), 4.08 (t, J=6.6 Hz, 2H), 3.62 (t, J=5.2 Hz, 2H), 2.78-2.50 (m, 10H), 2.35-2.22 (m, 4H), 2.00-1.88 (m, 2H), 1.57 (ddd, J=28.9, 13.5, 4.5 Hz, 14H), 1.38-1.20 (m, 42H), 0.88 (t, J=6.8 Hz, 9H).

¹³C NMR (101 MHz, CDCl₃) δ 173.83 (s), 173.60 (s), 140.60 (s), 138.33 (s), 128.44 (s), 128.24 (s), 77.36 (s), 77.04 (s), 76.72 (s), 74.15 (s), 63.69 (s), 57.95 (s), 55.83 (s), 53.95 (s), 35.56 (s), 34.65 (s), 34.21 (d, J=13.0 Hz), 31.81 (d, J=12.3 Hz), 31.54 (s), 30.32 (s), 29.52 (d, J=3.1 Hz), 29.34-28.94 (m), 27.13 (d, J=2.5 Hz), 26.31 (s), 25.33 (s), 24.99 (d, J=15.7 Hz), 22.64 (d, J=5.7 Hz), 14.11 (s).

### P. Compound SW-II-138-1

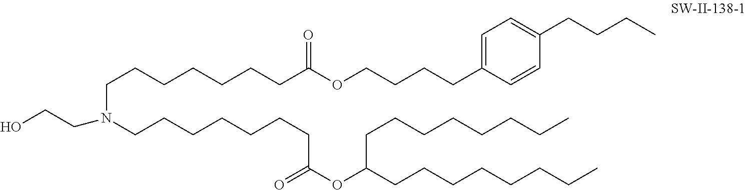 [SMILES: CCCCCCCCC(CCCCCCCC)OC(=O)CCCCCCCN(CCO)CCCCCCCC(=O)OCCCCc1ccc(CCCC)cc1]

### 1. Synthesis of Compound 2

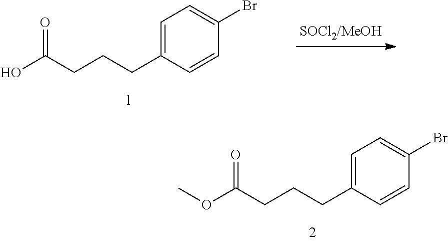

Compound 1 (4 g, 16.46 mmol, 1.0 eq.) was dissolved in MeOH (40 mL), cooled to 0° C. and SOCl₂ (3.9 g, 32.92 mmol, 2.0 eq) was added dropwise. Then the reaction was carried out at room temperature for 1 hour. TLC (PE/EA=5/1) showed that the starting material was consumed and the desired product was formed. The system was directly spin-dried under reduced pressure, NaHCO₃ (70 mL) solution was added to the residue, and extracted with EA (80 mL×3). The organic phases were combined and washed with saturated brine (2×30 mL), dried over anhydrous Na₂SO₄, filtered and spin-dried under reduced pressure. The residue was purified by silica gel chromatography, eluting with PE/EA (1/0-5:1, v/v), to obtain compound 2 (4.1 mg, 95%) as a yellow oil.

### 2. Synthesis of Compound 4

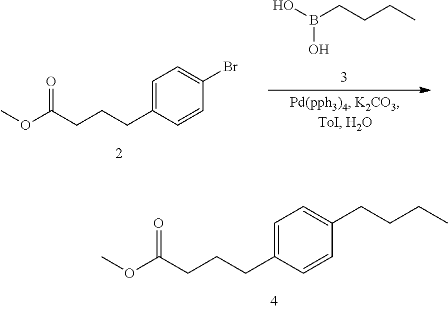

Compound 2 (500 mg, 1.95 mmol, 1.0 eq.), Compound 3 (239 mg, 2.34 mmol, 1.2 eq.), Pd(PPh₃)₄ (225 mg, 0.19 mmol, 0.1 eq) and K₂CO₃ (808 g, 5.85 mmol, 3.0 eq.) were dissolved in toluene (5.0 mL), then water (1 mL) was added. Then the reaction was carried out at 110° C. for 3 hours under nitrogen. TLC (PE/EA=5/1) showed that the starting material was consumed and the desired product was formed. The reaction was quenched by adding H₂O (70 mL), and extracted with EA (80 mL×3). The organic phases were combined and washed with saturated brine (2×30 mL), dried over anhydrous Na₂SO₄, filtered and spin-dried under reduced pressure. The residue was purified using a silica gel chromatography, eluting with PE/EA (1/0-5:1, v/v), to obtain compound 4 (320 mg, 70%) as a yellow oil.

### 3. Synthesis of Compound 5

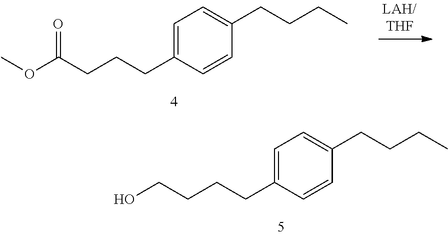 [SMILES: CCCCc1ccc(CCCC(=O)OC)cc1 ; CCCCc1ccc(CCCCO)cc1]

Compound 4 (300 mg, 1.28 mmol, 1.0 eq.) was dissolved in THF (4.0 mL), and LAH (97 mg, 2.56 mmol, 2.0 eq) was added at 0° C. Then the reaction was carried out at room temperature for 2 hours under nitrogen. TLC (PE/EA=5/1) showed that the starting material was consumed and the desired product was formed. The reaction was quenched by adding HCl (1M, 4 mL) solution and H₂O (10 mL), and extracted with EA (50 mL×3). The organic phase was washed with saturated brine (2×30 mL), dried over anhydrous Na₂SO₄, filtered and spin-dried under reduced pressure. The residue was purified by silica gel chromatography, eluting with PE/EA (1/0-5:1, v/v), to obtain compound 5 (224 mg, 84.8%) as a yellow oil.

### 4. Synthesis of Compound 7

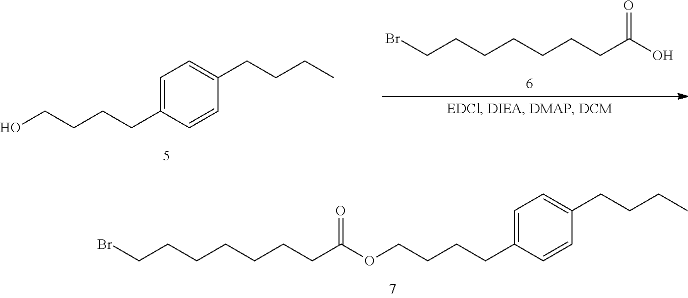 [SMILES: O=C(O)CCCCCCCBr ; CCCCc1ccc(CCCCO)cc1 ; CCCCc1ccc(CCCCOC(=O)CCCCCCCBr)cc1]

Compound 7 (224 mg, 1.09 mmol, 1.0 eq.) was dissolved in DCM (3.0 mL), and then compound 6 (290 mg, 1.30 mmol, 1.2 eq.), EDCI (415 mg, 2.17 mmol, 2.0 eq.), DIEA (561 mg, 4.35 mmol, 4.0 eq.) and DMAP (53 mg, 0.43 mmol, 0.4 eq.) were added. Then, the reaction was carried out overnight at room temperature under nitrogen. TLC (PE/EA=30/1) showed that the starting material was consumed and the desired product was formed. The reaction was quenched with HCl (1M) solution, adjusted to pH=4-6, and extracted with DCM (80 mL×3). The combined organic phase was washed with saturated brine (2×30 mL), dried over anhydrous Na₂SO₄, filtered and spin-dried under reduced pressure. The residue was purified by silica gel chromatography, eluting with PE/EA (1/0-30:1, v/v), to obtain compound 7 (208 mg, 46.7%) as a colorless oil.

### 5. Synthesis of SW-II-138-1

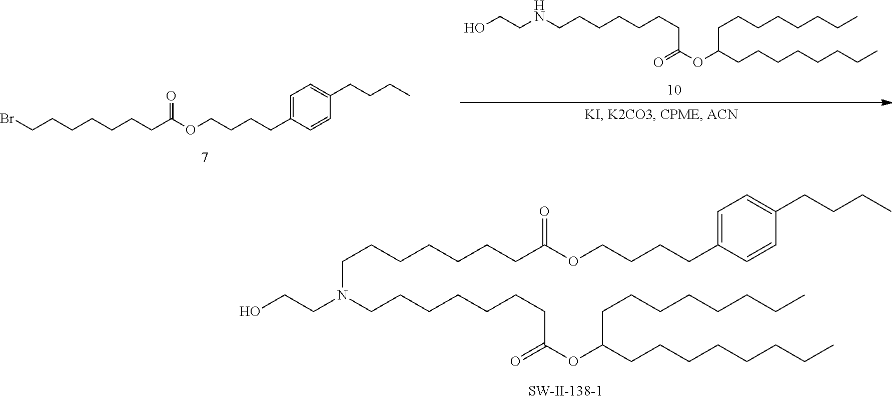 [SMILES: CCCCCCCCC(CCCCCCCC)OC(=O)CCCCCCCNCCO ; CCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCC)cc1]

Compound 10 (110 mg, 0.25 mmol, 1 eq.), compound 7 (153 mg, 0.37 mmol, 1.5 eq), KI (83 mg, 0.50 mmol, 2.0 eq) and CPME (2 mL) were dissolved in MeCN (2 mL), then K₂CO₃ (172 mg, 1.25 mmol, 5.0 eq) was added. The reaction was carried out overnight at 90° C. under nitrogen. TLC (DCM/MeOH=10/1) showed that the starting material was consumed and the desired product was formed. The reaction was directly spin-dried under reduced pressure. The residue was purified by silica gel chromatography, eluting with DCM/MeOH (1/0-10:1, v/v), to obtain a pale yellow oily compound (65 mg, 32%, SW-II-138-1).

LCMS: Rt: 1.684 min; MS m/z (ELSD): 772.4 [M+H]⁺;

HPLC: 96.56% purity at ELSD; RT=6.346 min.

¹H NMR (400 MHz, CDCl₃) δ 7.09 (s, 4H), 4.86 (s, 1H), 4.09 (d, J=6.0 Hz, 2H), 3.97 (s, 2H), 3.07 (d, J=38.8 Hz, 6H), 2.69-2.51 (m, 4H), 2.28 (td, J=7.3, 3.6 Hz, 4H), 1.79 (s, 4H), 1.70-1.46 (m, 16H), 1.42-1.17 (m, 37H), 0.90 (dt, J=13.6, 7.2 Hz, 9H).

¹³C NMR (101 MHz, CDCl₃) δ 173.80 (s), 173.53 (s), 140.32 (s), 139.13 (s), 128.28 (d, J=13.6 Hz), 77.43 (s), 77.11 (s), 76.80 (s), 74.21 (s), 64.22 (s), 56.85 (s), 55.98 (s), 53.93 (s), 35.22 (s), 35.01 (s), 34.54 (s), 34.14 (d, J=5.6 Hz), 33.71 (s), 31.85 (s), 29.50 (d, J=2.8 Hz), 29.22 (s), 29.12-28.60 (m), 28.26 (s), 27.78 (s), 26.70 (d, J=4.4 Hz), 25.31 (s), 24.82 (d, J=17.6 Hz), 24.28 (s), 22.65 (s), 22.37 (s), 14.03 (d, J=15.2 Hz).

### Q. Compound SW-II-138-2

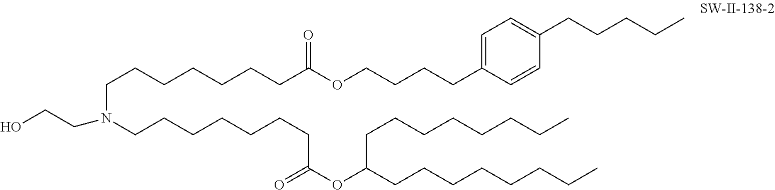 [SMILES: CCCCCCCCC(CCCCCCCC)OC(=O)CCCCCCCN(CCO)CCCCCCCC(=O)OCCCCc1ccc(CCCCC)cc1]

### 1. Synthesis of Compound 3

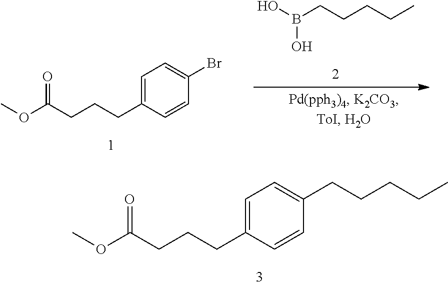

Compound 1 (500 mg, 1.95 mmol, 1.0 eq.), Compound 2 (271 mg, 2.34 mmol, 1.2 eq.), Pd(PPh₃)₄ (225 mg, 0.20 mmol, 0.1 eq) and K₂CO₃ (809 g, 5.86 mmol, 3.0 eq.) were dissolved in toluene (5.0 mL), then water (1 mL) was added. The reaction was carried out at 110° C. for 3 hours under nitrogen. TLC (PE/EA=5/1) showed that the starting material was consumed and the desired product was formed. The reaction was quenched with water (70 mL) and extracted with EA (80 mL×3). The organic phases were combined and washed with saturated brine (2×30 mL), dried over anhydrous Na₂SO₄, filtered and spin-dried under reduced pressure. The residue was purified by silica gel chromatography, eluting with PE/EA (1/0-30:1, v/v), to obtain compound 3 (320 mg, 70%) as a colorless oil.

### 2. Synthesis of Compound 4

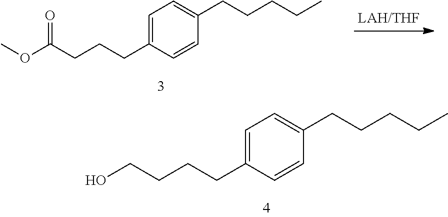 [SMILES: CCCCCc1ccc(CCCC(=O)OC)cc1 ; CCCCCc1ccc(CCCCO)cc1]

Compound 3 (320 mg, 1.29 mmol, 1.0 eq.) was dissolved in THF (3.0 mL), LAH (67 mg, 1.77 mmol, 2.0 eq) was added at 0° C., and then the reaction was carried out at room temperature for 2 hours under nitrogen. TLC (PE/EA=5/1) showed that the starting material was consumed and the desired product was formed. The reaction was quenched with HCl (1 M, 2 mL) solution and H₂O (10 mL), and extracted with EA (50 mL×3). The combined organic phase was washed with saturated brine (2×30 mL), dried over anhydrous Na₂SO₄, filtered and spin-dried under reduced pressure. The residue was purified by silica gel chromatography, eluting with PE/EA (1/0-30:1, v/v), to obtain compound 4 (180 mg, 64%) as a colorless oil.

### 3. Synthesis of Compound 6

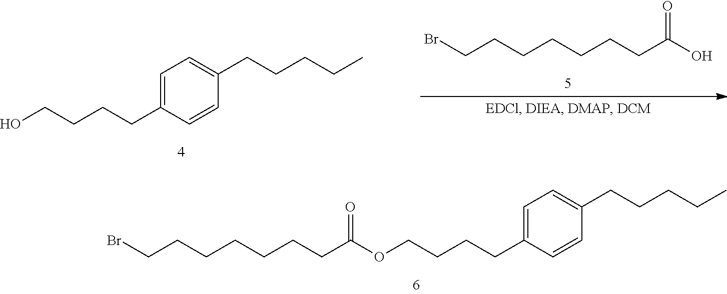 [SMILES: O=C(O)CCCCCCCBr ; CCCCCc1ccc(CCCCO)cc1 ; CCCCCc1ccc(CCCCOC(=O)CCCCCCCBr)cc1]

Compound 4 (180 mg, 0.82 mmol, 1.0 eq.) was dissolved in DCM (3.0 mL), and then Compound 5 (245 mg, 1.10 mmol, 1.2 eq.), EDCI (347 mg, 1.82 mmol, 2.0 eq.), DIEA (470 mg, 3.63 mmol, 4.0 eq.) and DMAP (45 mg, 0.36 mmol, 0.4 eq.) were added. Then, the reaction was carried out at room temperature overnight under nitrogen. TLC (PE/EA=30/1) showed that the consumption of starting material was completed and the desired product was formed. The reaction was quenched with HCl (1M) solution and adjusted to PH=5-6, and extracted with DCM (80 mL×3). The combined organic phase was washed with saturated brine (2×30 mL), dried over anhydrous Na₂SO₄, filtered and spin-dried under reduced pressure. The residue was purified by silica gel chromatography, eluting with PE/EA (1/0-30:1, v/v), to obtain compound 6 (220 mg, 63.6%) as a colorless oil.

### 4. Synthesis of SW-II-138-2

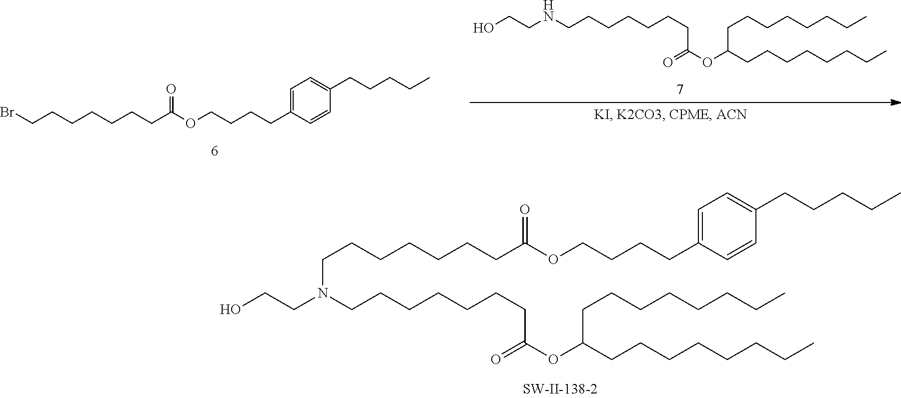 [SMILES: CCCCCCCCC(CCCCCCCC)OC(=O)CCCCCCCNCCO ; CCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCC)cc1]

Compound 6 (158 mg, 0.37 mmol, 1.5 eq.), Compound 7 (110 mg, 0.25 mmol, 1.0 eq), KI (83 mg, 0.50 mmol, 2.0 eq) and CPME (2 mL) were dissolved in MeCN (2 mL), then K₂CO₃ (172 mg, 1.25 mmol, 5.0 eq) was added. Then, the reaction was carried out at 90° C. overnight under nitrogen. TLC (DCM/MeOH=10/1) showed that the starting material was consumed and the desired product was formed. The reaction was directly spin-dried under reduced pressure, and the residue was purified by silica gel chromatography, eluting with DCM/MeOH (1/0-10:1, v/v), to obtain the colorless oily target product (100 mg, 51%, SW-II-138-2).

LCMS: Rt: 1.834 min; MS m/z (ELSD): 786.4 [M+H]⁺;

HPLC: 99.20% purity at ELSD; RT=7.990 min.

¹H NMR (400 MHz, CDCl₃) δ 7.00 (s, 4H), 4.88-4.73 (m, 2H), 4.00 (t, J=5.6 Hz, 2H), 3.81-3.54 (m, 2H), 3.00-2.81 (m, 2H), 2.81-2.65 (m, 4H), 2.50 (dd, J=16.4, 8.4 Hz, 4H), 2.20 (td, J=7.6, 3.2 Hz, 4H), 1.56 (ddd, J=18.4, 10.4, 5.2 Hz, 13H), 1.43 (d, J=5.6 Hz, 4H), 1.34-1.07 (m, 40H), 0.81 (dt, J=11.2, 5.6 Hz, 9H).

¹³C NMR (101 MHz, CDCl₃) δ 173.78 (s), 173.52 (s), 140.32 (s), 139.11 (s), 128.25 (d, J=11.6 Hz), 77.49 (s), 77.17 (s), 76.85 (s), 74.14 (s), 64.17 (s), 57.25 (s), 55.82 (s), 53.85 (s), 35.50 (s), 35.01 (s), 34.56 (s), 34.14 (d, J=7.2 Hz), 31.84 (s), 31.53 (s), 31.23 (s), 29.49 (d, J=2.8 Hz), 29.21 (s), 28.94 (dd, J=6.4, 4.4 Hz), 28.25 (s), 27.77 (s), 26.84 (d, J=4.4 Hz), 25.30 (s), 25.25-24.59 (m), 22.59 (d, J=11.2 Hz), 14.05 (d, J=7.6 Hz).

### R. Compound SW-II-138-3

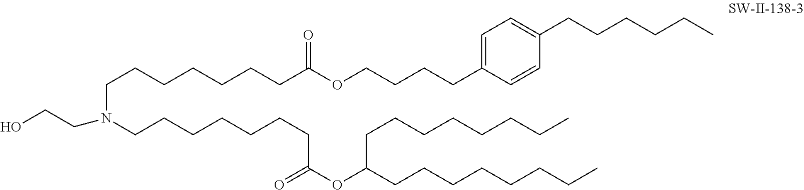 [SMILES: CCCCCCCCC(CCCCCCCC)OC(=O)CCCCCCCN(CCO)CCCCCCCC(=O)OCCCCc1ccc(CCCCCC)cc1]

### 1. Synthesis of Compound 3

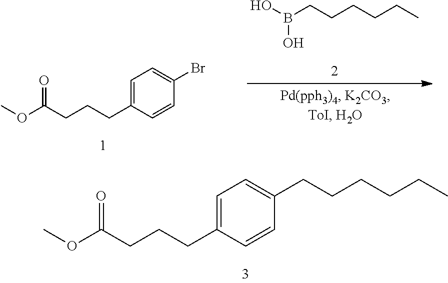

Compound 1 (500 mg, 1.95 mmol, 1.0 eq.), Compound 2 (305 mg, 2.34 mmol, 1.2 eq.), Pd(PPh₃)₄ (225 mg, 0.20 mmol, 0.1 eq) and K₂CO₃ (809 g, 5.86 mmol, 3.0 eq.) were dissolved in toluene (5.0 mL), and then water (1 mL) was added. Then, the reaction was carried out at 110° C. for 3 hours under nitrogen. TLC (PE/EA=5/1) showed that the starting material was consumed and the desired compound was formed. The reaction was quenched with water (80 mL) and extracted with EA (80 mL×3). The organic phases were combined and washed with saturated brine (2×40 mL), dried over anhydrous Na₂SO₄, filtered and spin-dried under reduced pressure. The residue was purified by silica gel chromatography, eluting with PE/EA (1/0-5:1, v/v), to obtain compound 3 (260 mg, 51.3%) as a colorless oil.

### 2. Synthesis of Compound 4

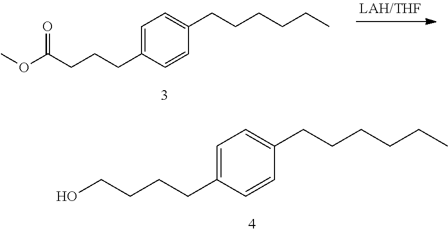 [SMILES: CCCCCCc1ccc(CCCC(=O)OC)cc1 ; CCCCCCc1ccc(CCCCO)cc1]

Compound 3 (260 mg, 0.99 mmol, 1.0 eq.) was dissolved in THF (4.0 mL), and LAH (75 mg, 1.98 mmol, 2.0 eq) was added at 0° C. Then, the reaction was carried out at room temperature for 2 hours under nitrogen. TLC (PE/EA=5/1) showed that the reaction of the starting material was completed and the desired compound was formed. The reaction was quenched with HCl (1 M, 4 mL) solution and H₂O (20 mL), and extracted with EA (50 mL×3). The combined organic phase was washed with saturated brine (2×30 mL), dried over anhydrous Na₂SO₄, filtered and spin-dried under reduced pressure. The residue was purified by silica gel chromatography, eluting with PE/EA (1/0-5:1, v/v), to give compound 4 (230 mg, 98%) as a colorless oil.

### 3. Synthesis of Compound 6

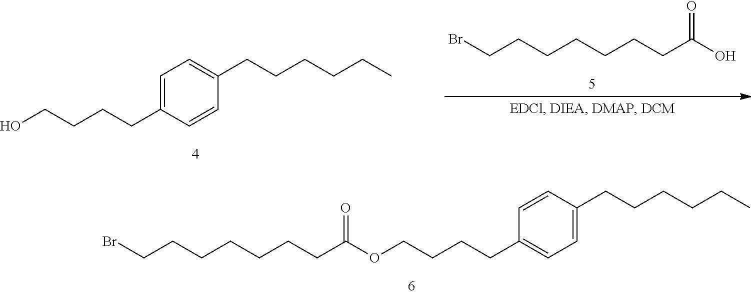 [SMILES: O=C(O)CCCCCCCBr ; CCCCCCc1ccc(CCCCO)cc1 ; CCCCCCc1ccc(CCCCOC(=O)CCCCCCCBr)cc1]

Compound 4 (240 mg, 1.03 mmol, 1.0 eq.) was dissolved in DCM (4.0 mL), and then compound 5 (275 mg, 1.23 mmol, 1.2 eq.), EDCI (392 mg, 2.07 mmol, 2.0 eq.), DIEA (530 mg, 4.10 mmol, 4.0 eq.) and DMAP (50 mg, 0.41 mmol, 0.4 eq.) were in turn added. Then the reaction was carried out overnight at room temperature under nitrogen. TLC (PE/EA=20/1) showed that the starting material was consumed and the desired compound was formed. The reaction was quenched with HCl (1 M) and adjusted to pH=5-6, and extracted with DCM (80 mL×3). The combined organic phase was washed with saturated brine (2×30 mL), dried over anhydrous Na₂SO₄, filtered and spin-dried under reduced pressure. The residue was purified by silica gel chromatography, eluting with PE/EA (1/0-20:1, v/v), to obtain compound 6 (180 mg, 40.9%) as a colorless oil.

### 4. Synthesis of SW-II-138-3

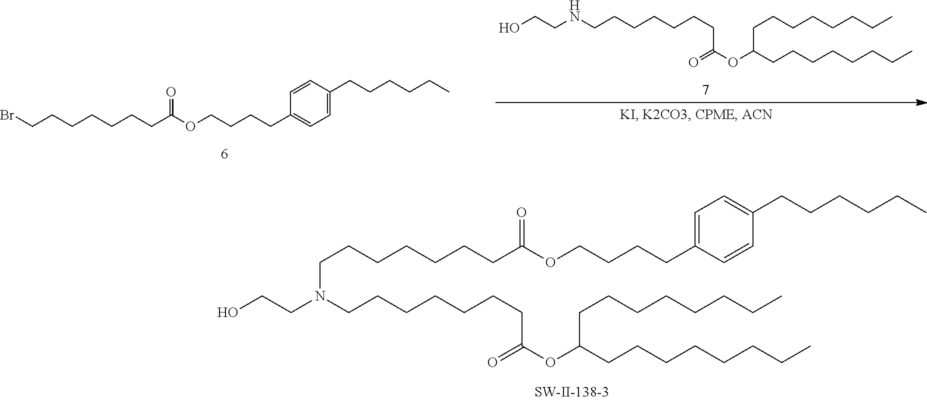 [SMILES: CCCCCCCCC(CCCCCCCC)OC(=O)CCCCCCCNCCO ; CCCCCCc1ccc(CCCCOC(=O)CCCCCCCBr)cc1 ; CCCCCCCCC(CCCCCCCC)OC(=O)CCCCCCCN(CCO)CCCCCCCC(=O)OCCCCc1ccc(CCCCCC)cc1]

Compound 6 (164 mg, 0.37 mmol, 1 eq.), compound 7 (110 mg, 0.24 mmol, 1.0 eq), KI (83 mg, 0.49 mmol, 2.0 eq) and CPME (2 mL) were dissolved in MeCN (2 mL), and then K₂CO₃ (172 mg, 1.24 mmol, 5.0 eq) was added. Then, the reaction was carried out at 90° C. overnight under nitrogen. TLC (DCM/MeOH=10/1) showed that the starting material was consumed and the desired product was formed. The reaction was directly spin-dried under reduced pressure. The residue was purified by silica gel chromatography, eluting with DCM/MeOH (1/0-10:1, v/v), to obtain the colorless oily target product (108 mg, 52.76%, SW-II-138-3).

LCMS: Rt: 2.007 min; MS m/z (ELSD): 800.4 [M+H]⁺;

HPLC: 97.95% purity at ELSD; RT=9.455 min.

¹H NMR (400 MHz, CDCl₃) δ 7.08 (s, 4H), 4.86 (p, J=6.4 Hz, 1H), 4.08 (s, 2H), 3.60 (t, J=5.2 Hz, 3H), 2.76-2.42 (m, 10H), 2.28 (td, J=7.6, 2.8 Hz, 4H), 1.70-1.42 (m, 18H), 1.28 (d, J=20.0 Hz, 41H), 0.88 (t, J=6.8 Hz, 9H).

¹³C NMR (101 MHz, CDCl₃) δ 173.85 (s), 173.59 (s), 140.39 (s), 139.15 (s), 128.27 (d, J 25=12.0 Hz), 77.38 (s), 77.07 (s), 76.75 (s), 74.13 (s), 64.17 (s), 58.06 (s), 55.75 (s), 53.92 (s), 35.57 (s), 35.03 (s), 34.66 (s), 34.22 (d, J=13.2 Hz), 31.81 (d, J=12.4 Hz), 31.54 (s), 29.52 (d, J=2.9 Hz), 29.34-28.95 (m), 28.29 (s), 27.79 (s), 27.16 (d, J=3.6 Hz), 26.50 (s), 25.32 (s), 24.99 (d, J=17.6 Hz), 22.64 (d, J=5.6 Hz), 14.10 (s).

### S. Compound SW-II-139-1

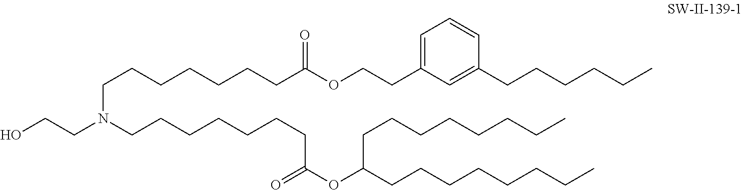 [SMILES: CCCCCCCCC(CCCCCCCC)OC(=O)CCCCCCCN(CCO)CCCCCCCC(=O)OCCc1cccc(CCCCCC)c1]

### 1. Synthesis of Compound 3

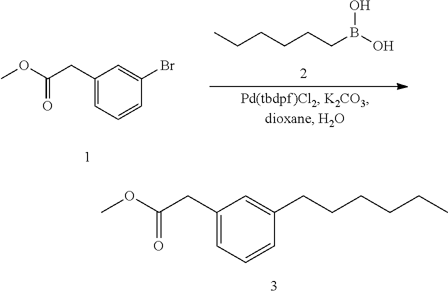

To a mixture of compound 1 (1 g, 4.37 mmol, 1 eq.) and compound 2 (852 g, 6.55 mmol, 1.5 eq) in 1,4-dioxane/water (10 mL/1 mL) was added Pd(dtbpf)Cl₂ (286 mg, 0.437 mmol, 0.1 eq.) and potassium carbonate (1.8 g, 13.11 mmol, 3 eq). The mixture was stirred at 100° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to give compound 3 (691 mg, 68%) as a colorless oil.

### 2. Synthesis of Compound 4

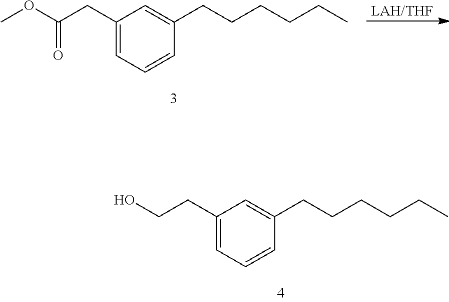

To a mixture of compound 3 (691 mg, 2.95 mmol, 1 eq.) in THF (7 mL) was added lithium aluminum hydride (3 mL, 2.95 mmol, 1 M in THF, 1 eq.) at 0° C. under N₂. The mixture was stirred at room temperature for 3 hours. TLC (PE/EA=5/1) indicated that the reaction was completed and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 4 (547 mg, 90%) as a colorless oil, which was used without further purification.

### 3. Synthesis of Compound 6

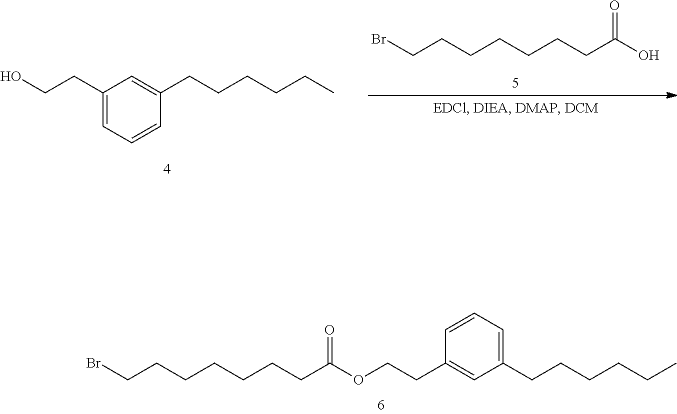

To a mixture of compound 4 (447 mg, 2.17 mmol, 1 eq.) and compound 5 (581 mg, 2.6 mmol, 1.2 eq.) in DCM (5 mL) was added EDCI (833 mg, 4.34 mmol, 2 eq.) and DMAP (106 mg, 0.87 mmol, 0.4 eq.). Then DIEA (1.12 g, 8.68 mmol, 4 eq.) was added. The reaction mixture was stirred at room temperature under nitrogen for 16 hours. TLC (petroleum ether/ethyl acetate=15/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (455 mg, 51%) as a colorless oil.

### 4. Synthesis of SW-II-139-1

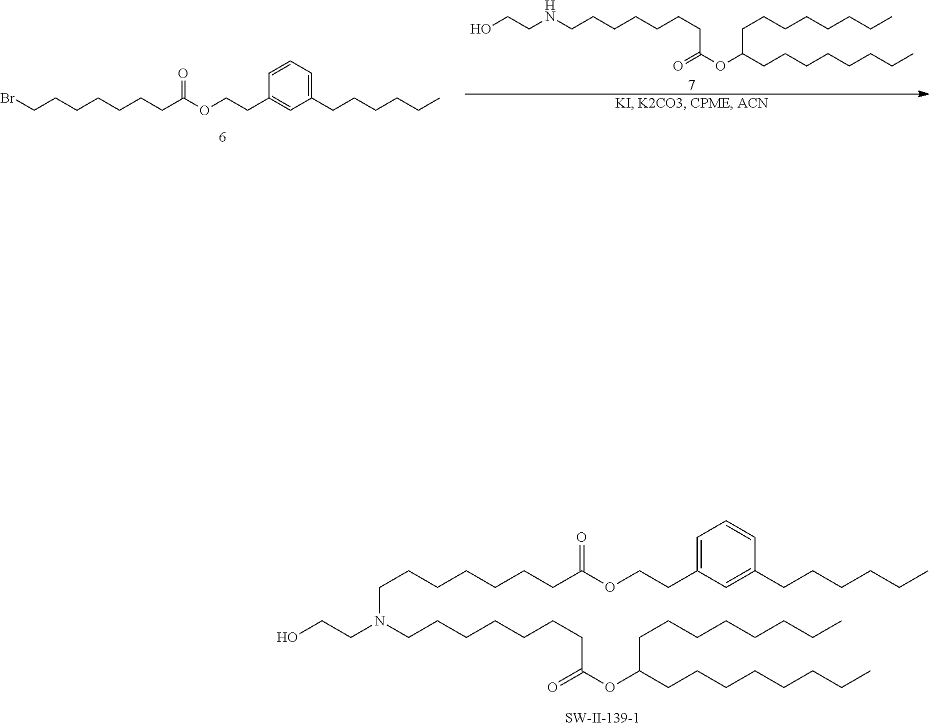

To a mixture of compound 6 (150 mg, 0.365 mmol, 1 eq.) and compound 7 (161 mg, 0.365 mmol, 1 eq.) in CPME/CH₃CN (2 mL/2 mL) was added potassium carbonate (252 mg, 1.825 mmol, 6 eq.) and potassium iodide (121 mg, 0.73 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-139-1 (54.53 mg, 19%) as a yellow oil.

LCMS: Rt: 1.521 min; MS m/z (ELSD): 772.4 [M+H]⁺;

HPLC: 99.637% purity at ELSD; RT=12.347 min.

¹H NMR (400 MHz, CDCl₃) δ 7.20 (t, J=7.7 Hz, 1H), 7.03 (t, J=6.8 Hz, 3H), 4.94-4.78 (m, 1H), 4.27 (t, J=7.2 Hz, 2H), 3.65 (t, J=5.1 Hz, 2H), 2.90 (t, J=7.2 Hz, 2H), 2.73 (t, J=4.9 Hz, 2H), 2.67-2.41 (m, 6H), 2.28 (td, J=7.5, 2.7 Hz, 4H), 1.67-1.45 (m, 14H), 1.41-1.19 (m, 42H), 0.88 (dd, J=7.9, 5.7 Hz, 9H).

¹³C NMR (101 MHz, CDCl₃) δ 173.65 (d, J=11.3 Hz), 143.17 (s), 137.67 (s), 129.04 (s), 128.34 (s), 126.61 (s), 126.11 (s), 77.30 (d, J=11.6 Hz), 77.04 (s), 76.72 (s), 74.16 (s), 64.85 (s), 57.88 (s), 55.93 (s), 53.97 (s), 35.94 (s), 35.13 (s), 34.64 (s), 34.20 (d, J=10.5 Hz), 31.80 (d, J=13.7 Hz), 31.50 (s), 29.52 (d, J=2.9 Hz), 29.34-28.92 (m), 27.08 (d, J=3.9 Hz), 26.10 (s), 25.33 (s), 25.05 (s), 24.82 (s), 22.64 (d, J=6.5 Hz), 14.11 (s).

### T. Compound SW-II-139-2

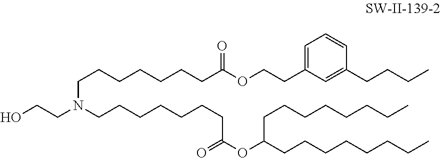 [SMILES: CCCCCCCCC(CCCCCCCC)OC(=O)CCCCCCCN(CCO)CCCCCCCC(=O)OCCc1cccc(CCCC)c1]

### 1. Synthesis of Compound 3

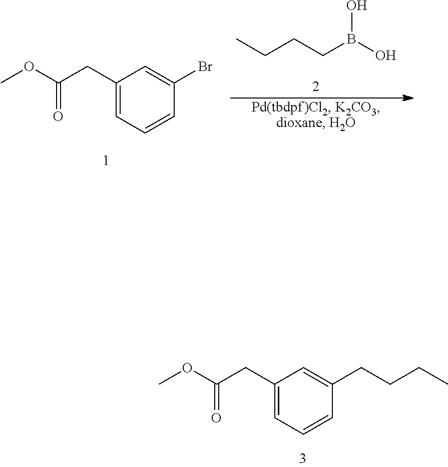

To a mixture of compound 1 (1 g, 4.37 mmol, 1 eq.) and compound 2 (668 mg, 6.55 mmol, 1.5 eq) in 1,4-dioxane/water (10 mL/1 mL) was added Pd(dtbpf)Cl₂ (286 mg, 0.437 mmol, 0.1 eq.) and potassium carbonate (1.8 g, 13.11 mmol, 3 eq). The mixture was stirred at 100° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to obtain compound 3 (605 mg, 67%) as a colorless oil.

### 2. Synthesis of Compound 4

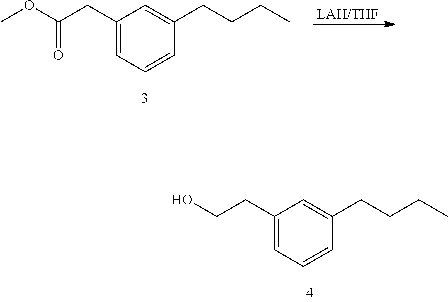

To a mixture of compound 3 (605 mg, 2.94 mmol, 1 eq.) in THF (7 mL) was added lithium aluminum hydride (3 mL, 2.94 mmol, 1 M in THF, 1 eq.) 0° C. under N₂. The mixture was stirred at room temperature for 3 hours. TLC (PE/EA=5/1) indicated that the reaction was completed and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 4 (534 mg, >100%) as a colorless oil, which was used without further purification.

### 3. Synthesis of Compound 6

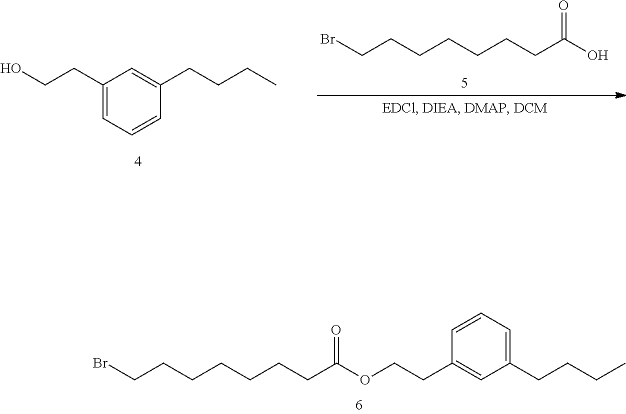

To a mixture of compound 4 (434 mg, 2.44 mmol, 1 eq.) and compound 5 (652 mg, 2.93 mmol, 1.2 eq.) in DCM (5 mL) was added EDCI (937 mg, 4.88 mmol, 2 eq.) and DMAP (119 mg, 0.976 mmol, 0.4 eq.). Then DIEA (1.259 g, 9.76 mmol, 4 eq.) was added. The reaction mixture was stirred at room temperature under nitrogen for 16 hours. TLC (petroleum ether/ethyl acetate=15/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (355 mg, 38%) as a colorless oil.

### 4. Synthesis of SW-II-139-2

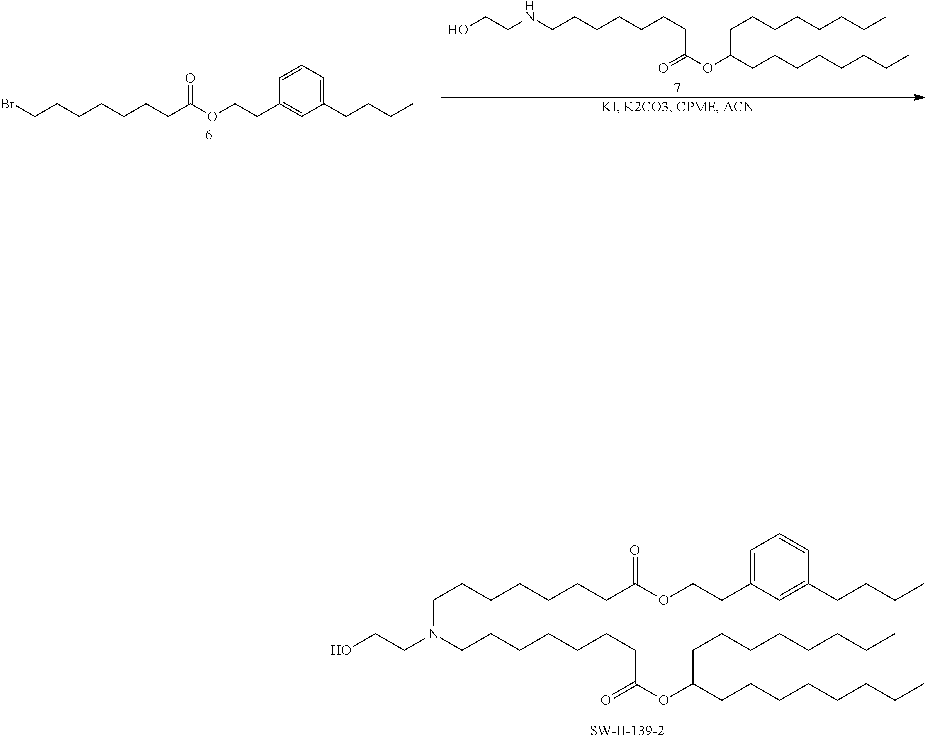

To the mixture of compound 6 (122 mg, 0.319 mmol, 1 eq.) and compound 7 (140 mg, 0.319 mmol, 1 eq.) in CPME/CH₃CN (2 mL/2 mL) was added potassium carbonate (220 mg, 1.595 mmol, 5 eq.) and potassium iodide (106 mg, 0.638 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-139-2 (45.48 mg, 19%) as a yellow oil.

LCMS: Rt: 1.346 min; MS m/z (ELSD): 744.3 [M+H]⁺;

HPLC: 97.994% purity at ELSD; RT=11.235 min.

¹H NMR (400 MHz, CDCl₃) δ 7.20 (t, J=7.8 Hz, 1H), 7.03 (t, J=7.6 Hz, 3H), 4.91-4.81 (m, 1H), 4.27 (t, J=7.2 Hz, 2H), 3.89-3.75 (m, 2H), 2.99-2.79 (m, 7H), 2.64-2.48 (m, 2H), 2.28 (td, J=7.5, 3.1 Hz, 4H), 1.74-1.08 (m, 53H), 0.90 (dt, J=13.6, 7.2 Hz, 9H).

¹³C NMR (101 MHz, CDCl₃) δ 173.60 (d, J=11.7 Hz), 143.13 (s), 137.65 (s), 129.06 (s), 128.34 (s), 126.64 (s), 126.11 (s), 77.30 (d, J=11.4 Hz), 77.04 (s), 76.72 (s), 74.22 (s), 64.88 (s), 57.28 (s), 56.55 (s), 54.11 (s), 35.60 (s), 35.12 (s), 34.56 (s), 34.15 (d, J=4.0 Hz), 33.68 (s), 31.86 (s), 29.52 (d, J=2.8 Hz), 29.24 (s), 28.91 (dd, J=7.0, 4.2 Hz), 26.81 (d, J=3.9 Hz), 25.33 (s), 25.12-24.98 (m), 24.83 (d, J=22.2 Hz), 22.67 (s), 22.40 (s), 14.04 (d, J=14.4 Hz).

### U. Compound SW-II-140-1

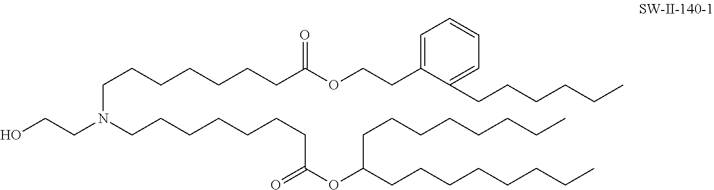 [SMILES: CCCCCCCCC(CCCCCCCC)OC(=O)CCCCCCCN(CCO)CCCCCCCC(=O)OCCc1ccccc1CCCCCC]

### 1. Synthesis of Compound 3

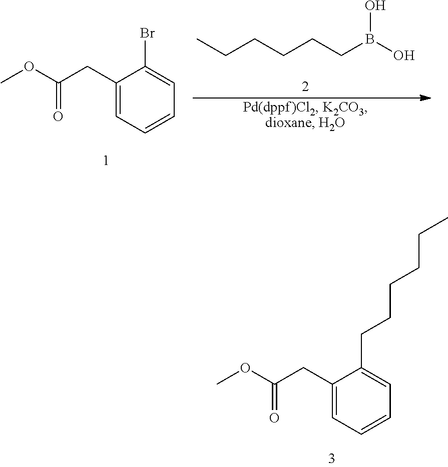

To a mixture of compound 1 (1 g, 4.37 mmol, 1 eq.) and compound 2 (852 g, 6.55 mmol, 1.5 eq) in 1,4-dioxane/water (10 mL/1 mL) was added Pd(dppf)Cl₂ (286 mg, 0.437 mmol, 0.1 eq.) and potassium carbonate (1.8 g, 13.11 mmol, 3 eq). The mixture was stirred at 100° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to obtain compound 3 (748 mg, 73%) as a colorless oil.

### 2. Synthesis of Compound 4

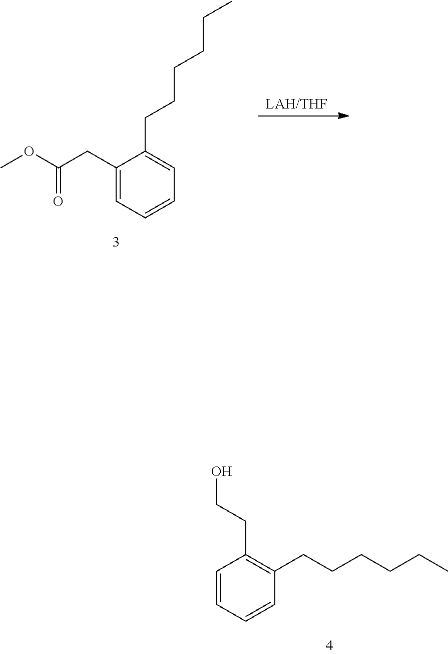 [SMILES: CCCCCCc1ccccc1CC(=O)OC ; CCCCCCc1ccccc1CCO]

To a mixture of compound 3 (748 mg, 3.2 mmol, 1 eq.) in THF (8 mL) was added lithium aluminum hydride (3.2 mL, 3.2 mmol, 1 M in THF, 1 eq.) under nitrogen at 0° C. The mixture was stirred at room temperature for 3 hours. TLC (PE/EA=5/1) indicated that the reaction was completed and new major spots were observed. The mixture was quenched with water (3 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 4 (493 mg, 75%) as a colorless oil, which was used without further purification.

### 3. Synthesis of Compound 6

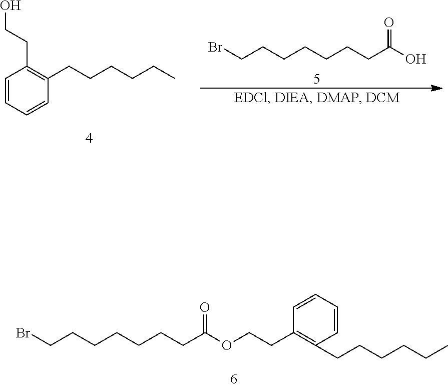

To a mixture of compound 4 (393 mg, 1.91 mmol, 1 eq.) and compound 5 (511 mg, 2.29 mmol, 1.2 eq.) in DCM (5 mL) was added EDCI (733 mg, 3.82 mmol, 2 eq.) and DMAP (93 mg, 0.76 mmol, 0.4 eq.). Then DIEA (986 mg, 7.64 mmol, 4 eq.) was added. The reaction mixture was stirred at room temperature under nitrogen for 16 hours. TLC (petroleum ether/ethyl acetate=15/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (327 mg, 42%) as a colorless oil.

### 4. Synthesis of SW-II-140-1

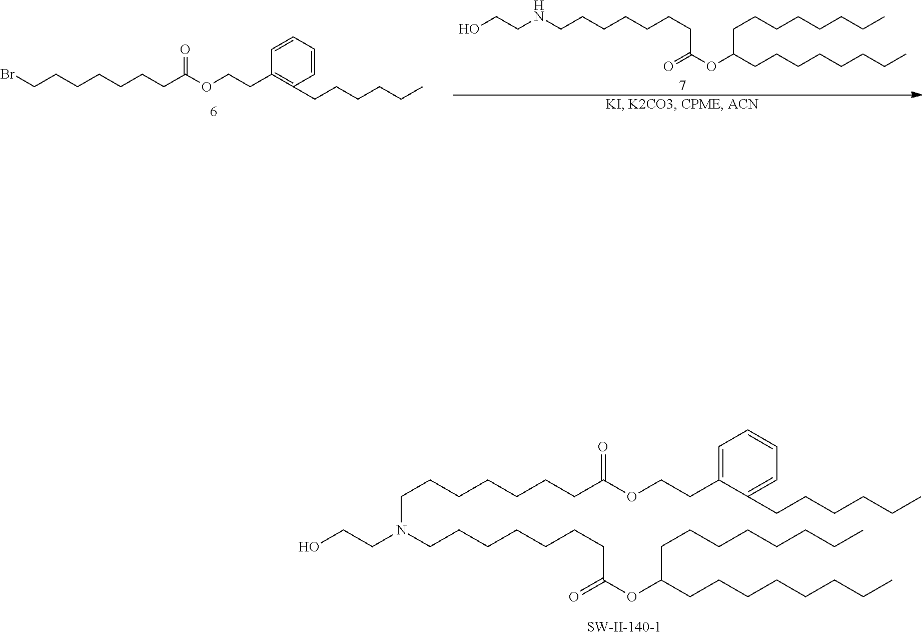

To a mixture of compound 6 (150 mg, 0.365 mmol, 1 eq.) and compound 7 (161 mg, 0.365 mmol, 1 eq.) in CPME/CH₃CN (2 mL/2 mL) was added potassium carbonate (302 mg, 2.19 mmol, 6 eq.) and potassium iodide (121 mg, 0.73 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-140-1 (180 mg, 64%) as a yellow oil.

LCMS: Rt: 1.568 min; MS m/z (ELSD): 772.4 [M+H]⁺;

HPLC: 98.053% purity at ELSD; RT=8.702 min.

¹H NMR (400 MHz, CDCl₃) δ 7.23-7.05 (m, 4H), 4.95-4.79 (m, 1H), 4.25 (t, J=7.4 Hz, 2H), 3.62 (t, J=4.8 Hz, 2H), 2.96 (dd, J=15.4, 8.0 Hz, 2H), 2.74-2.49 (m, 8H), 2.28 (dd, J=14.2, 7.2 Hz, 4H), 1.67-1.44 (m, 14H), 1.41-1.20 (m, 42H), 0.90 (dt, J=13.2, 7.1 Hz, 9H).

¹³C NMR (101 MHz, CDCl₃) δ 173.68 (d, J=10.2 Hz), 141.26 (s), 135.23 (s), 129.73 (s), 129.37 (s), 126.72 (s), 125.92 (s), 77.35 (s), 77.03 (s), 76.71 (s), 74.17 (s), 64.52 (s), 57.99 (s), 55.87 (s), 53.94 (s), 34.66 (s), 34.21 (d, J=11.5 Hz), 32.75 (s), 31.83 (d, J=9.8 Hz), 31.32 (s), 29.65-28.88 (m), 27.15 (d, J=3.7 Hz), 26.35 (s), 25.33 (s), 25.07 (s), 24.83 (s), 22.66 (d, J=3.4 Hz), 14.12 (s).

### V. Compound SW-II-140-2

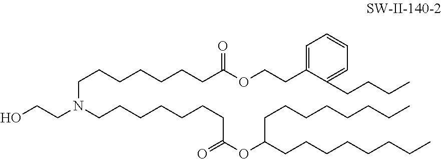 [SMILES: CCCCCCCCC(CCCCCCCC)OC(=O)CCCCCCCN(CCO)CCCCCCCC(=O)OCCc1ccccc1CCCC]

### 1. Synthesis of Compound 3

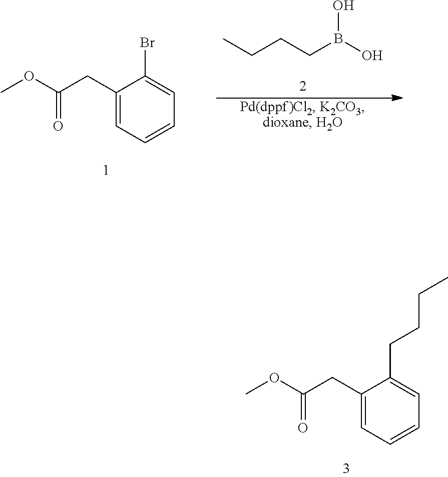

To a mixture of compound 1 (1 g, 4.37 mmol, 1 eq.) and compound 2 (668 g, 6.55 mmol, 1.5 eq) in 1,4-dioxane/water (10 mL/1 mL) was added Pd(dppf)Cl₂ (286 mg, 0.437 mmol, 0.1 eq.) and potassium carbonate (1.8 g, 13.11 mmol, 3 eq). The mixture was stirred at 100° C. overnight under nitrogen. TLC (PE/EA=20/1) showed that the reaction was completed and new major spots were observed. The mixture was extracted with ethyl acetate and washed with water. The organic layer was dried over anhydrous sodium sulfate, filtered, and concentrated in vacuo. The residue was purified by silica gel chromatography, eluting with PE/EA (1/0-20/1), to obtain compound 3 (406 mg, 45%) as a colorless oil.

### 2. Synthesis of Compound 4

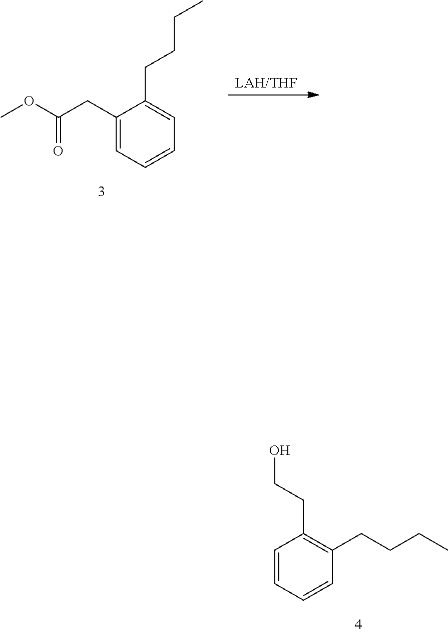

To a mixture of compound 3 (406 mg, 1.97 mmol, 1 eq.) in THF (5 mL) was added lithium aluminum hydride (2 mL, 1.97 mmol, 1 M in THF, 1 eq.) at 0° C. under N₂. The mixture was stirred at room temperature for 3 hours. TLC (PE/EA=5/1) indicated that the reaction was completed and new major spots were observed. The mixture was quenched with water (2 mL) and treated with 2N hydrochloric acid to adjust the pH between 6 and 7, extracted with ethyl acetate and washed with brine. The organic layer was dried over anhydrous sodium sulfate, filtered and concentrated in vacuo to obtain compound 4 (341 mg, 97%) as a colorless oil, which was used without further purification.

### 3. Synthesis of Compound 6

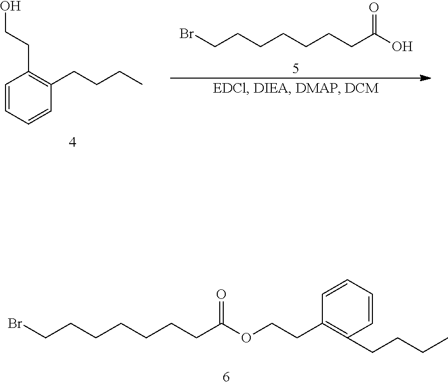

To a mixture of compound 4 (241 mg, 1.35 mmol, 1 eq.) and compound 5 (361 mg, 1.62 mmol, 1.2 eq.) in DCM (3 mL) was added EDCI (518 mg, 2.7 mmol, 2 eq.) and DMAP (66 mg, 0.54 mmol, 0.4 eq.). Then DIEA (697 mg, 5.4 mmol, 4 eq.) was added. The reaction mixture was stirred at room temperature under nitrogen for 16 hours. TLC (petroleum ether/ethyl acetate=15/1) showed that the desired product was formed. The reaction mixture was extracted with ethyl acetate and washed with water. The organic layer was dried over anhydrous sodium sulfate, filtered, and concentrated in vacuo. The residue was purified by silica gel chromatography, eluting with petroleum ether/ethyl acetate (1/0-20/1), to give compound 6 (185 mg, 32%) as a colorless oil.

### 4. Synthesis of SW-II-140-2

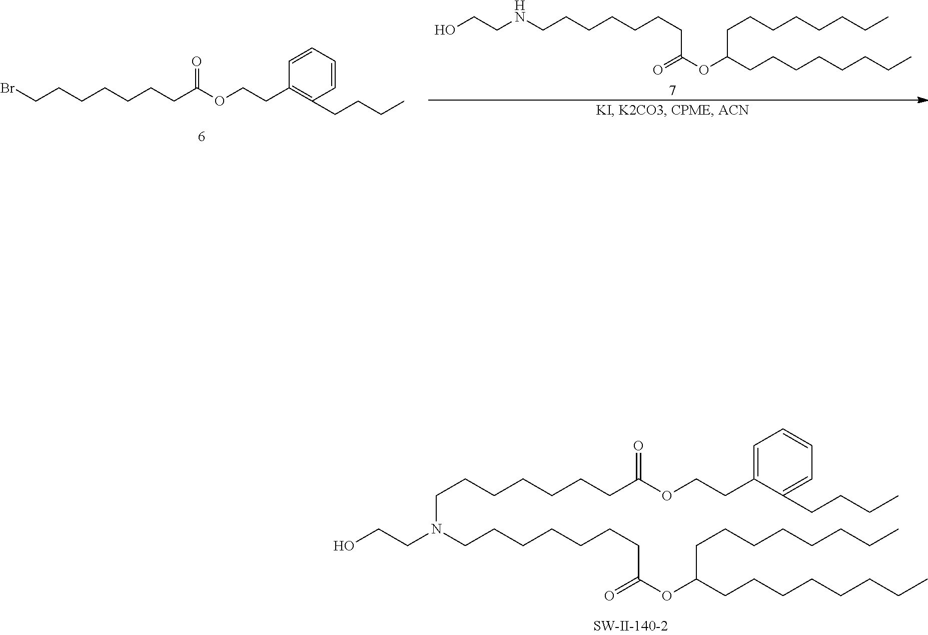

To a mixture of compound 6 (185 mg, 0.483 mmol, 1 eq.) and compound 7 (213 mg, 0.483 mmol, 1 eq.) in CPME/CH₃CN (2 mL/2 mL) was added potassium carbonate (400 mg, 2.898 mmol, 6 eq.) and potassium iodide (160 mg, 0.966 mmol, 2 eq.). After the addition, the mixture was stirred at 90° C. overnight under nitrogen. TLC (DCM/MeOH=10/1) showed that the reaction was completed and new major spots were observed. The mixture was extracted with ethyl acetate and washed with water. The organic layer was dried over anhydrous sodium sulfate, filtered, and concentrated under reduced pressure. The residue was purified by silica gel chromatography, eluting with DCM/MeOH (1/0-10:1, v/v), to give compound SW-II-140-2 (161 mg, 45%) as a yellow oil.

LCMS: Rt: 1.696 min; MS m/z (ELSD): 744.3 [M+H]⁺;

HPLC: 94.658% purity at ELSD; RT=5.938 min.

¹H NMR (400 MHz, CDCl₃) δ 7.22-7.03 (m, 4H), 4.94-4.78 (m, 1H), 4.25 (t, J=7.3 Hz, 2H), 3.70-3.54 (m, 2H), 2.96 (t, J=7.4 Hz, 2H), 2.77-2.41 (m, 8H), 2.28 (dd, J=14.3, 7.1 Hz, 4H), 1.65-1.18 (m, 52H), 0.91 (dt, J=13.3, 7.1 Hz, 9H).

¹³C NMR (101 MHz, CDCl₃) δ 173.67 (d, J=10.8 Hz), 141.22 (s), 135.23 (s), 129.73 (s), 129.39 (s), 126.72 (s), 125.92 (s), 77.36 (s), 77.04 (s), 76.72 (s), 74.17 (s), 64.52 (s), 57.92 (s), 55.92 (s), 53.96 (s), 34.66 (s), 34.21 (d, J=11.2 Hz), 33.51 (s), 32.44 (s), 31.83 (d, J=9.3 Hz), 29.53 (d, J=2.9 Hz), 29.14 (dd, J=11.3, 8.5 Hz), 27.12 (d, J=4.1 Hz), 26.23 (s), 25.33 (s), 25.06 (s), 24.82 (s), 22.73 (d, J=9.9 Hz), 14.08 (d, J=8.8 Hz).

### Example 2: Production of Nanoparticle Compositions

A. Production of Nanoparticle Compositions

In order to investigate safe and efficacious nanoparticle compositions for use in the delivery of therapeutic or prophylactics to cells, a range of formulations were prepared and tested.

Nanoparticles can be made with mixing processes such as microfluidics and T-junction mixing of two fluid streams, one of which contains the therapeutic or prophylactic and the other has the lipid components.

Lipid compositions were prepared by combining an obtained lipid, a phospholipid (such as DOPE or DSPC, obtainable from Cordenpharma), a PEG lipid (such as 1,2-dimyristoyl-sn-glycerol methoxypolyethylene glycol, also known as PEG-DMG, obtainable from Avanti Polar Lipids, Alabaster, Ala.), and a structural lipid (such as cholesterol, obtainable from Sigma-Aldrich, Taufkirchen, Germany) at concentrations of about 50 mM in ethanol.

The RNA used herein was an in vitro transcribed mRNA encoding luciferase (Luc) wherein each uridine was replaced with N1-methyl pseudouridine. For nanoparticle compositions including an RNA, solutions of the RNA at concentrations of 0.1 mg/ml in deionized water were diluted in 50 mM sodium citrate buffer at a pH between 3 and 4 to form a stock solution.

Preparation instrument: obtainable from Micronanobiologics (Shanghai) Technology Co., Ltd., model: Inano D.

LNP Preparation Steps:
1) Preparation of lipid solution: The lipids in proportion of ionizable lipid: phospholipid:cholesterol:mPEG2000-DMG=50:10:38.5:1.5 were dissolved in ethanol solution;
2) Preparation of LNP: 1 mL Luciferase mRNA and 3 mL lipid solution were inserted with syringe respectively into the microfluidic chip, setting the parameters as: volume: 12.0 mL, flow rate ratio: 3:1, total flow rate: 18 mL/min, temperature: 37.0° C., start waste 0.35 mL, and end waste 0.10 mL to obtain LNP solution;
3) Centrifugal ultrafiltration: 12 mL LNP solution and 12 mL ultrafiltration medium phosphate buffer were added to the ultrafiltration tube, setting the ultrafiltration parameters as: the centrifugal force: 3400 g, the centrifugal time: 60 min, the temperature: 4° C., and the number of cycles: 3 times, to obtain LNP.

B. Characterization of Nanoparticle Compositions

A Zetasizer Nano ZS (Malvern Instruments Ltd, Malvern, Worcestershire, UK) can be used to determine the particle size, the polydispersity index (PDI) and the zeta (ζ) potential of the nanoparticle compositions in 1×PBS in determining particle size and 15 mM PBS in determining zeta (ζ) potential.

Ultraviolet-visible spectroscopy can be used to determine the concentration of a therapeutic or prophylactic (e.g., RNA) in nanoparticle compositions. 100 μL of the diluted formulation in 1×PBS is added to 900 μL of a 4:1 (v/v) mixture of methanol and chloroform. After mixing, the absorbance spectrum of the solution was recorded, for example, between 230 nm and 330 nm on a DU 800 spectrophotometer (Beckman Coulter, Beckman Coulter, Inc., Brea, Calif.). The concentration of therapeutic or prophylactic in the nanoparticle composition can be calculated based on the extinction coefficient of the therapeutic or prophylactic used in the composition and on the difference between the absorbance at a wavelength of, for example, 260 nm and the baseline value at a wavelength of, for example, 330 nm.

For nanoparticle compositions including an RNA, a QUANT-IT™ RIBOGREENR®RNA assay (Invitrogen Corporation Carlsbad, Calif.) can be used to evaluate the encapsulation of an RNA by the nanoparticle composition. The samples were diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREENR reagent is diluted 1:100 in TE buffer and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

C. In Vivo Formulation Studies

In order to monitor how effectively various nanoparticle compositions, deliver therapeutic or prophylactics to targeted cells, different nanoparticle compositions including a particular therapeutic or prophylactic (for example, a modified or naturally occurring RNA such as an mRNA) were prepared and administered to rodent populations. Mice are intravenously, intramuscularly, intraarterially, or intratumorally administered a single dose including a nanoparticle composition with a formulation such as those provided in Examples 3. In some instances, mice may be made to inhale doses. Dose sizes may range from 0.001 mg/kg to 10 mg/kg, where 10 mg/kg describes a dose including 10 mg of a therapeutic or prophylactic in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. For nanoparticle compositions including mRNA, time courses of protein expression can also be evaluated. Samples collected from the rodents for evaluation may include blood, sera, and tissue (for example, muscle tissue from the site of an intramuscular injection and internal tissue); sample collection may involve sacrifice of the animals.

Nanoparticle compositions including mRNA were used in the evaluation of the efficacy and usefulness of various formulations for the delivery of therapeutic or prophylactics. Higher levels of protein expression induced by administration of a composition including an mRNA will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of the therapeutic or prophylactic by a given nanoparticle composition relative to other nanoparticle compositions or the absence thereof.

### Example 3: Optimization of Phospholipid and Sample Formulations

A variety of phospholipids in a lipid component of a nanoparticle composition were selected to optimize the formulation. A obtained lipid compound was selected for use in the nanoparticle composition and DSPC, DAPC, DPPC, DOPE, DMPE, DSPE and DPPE were selected as phospholipids. Additional phospholipids can also be evaluated. Nanoparticle compositions were evaluated for their size, polydispersity index (PDI), encapsulation efficiency, potential, cytotoxicity, Luc expression levels including in vitro cell expression and in vivo expression.

Initial studies were performed to investigate the delivery efficiency of nanoparticle compositions including various lipid compounds of the present invention. Nanoparticle compositions including a phospholipid as above mentioned, cholesterol as a structural lipid, PEG-DMG as a PEG lipid, an RNA, and a lipid compound selected from compounds SW-II-118, SW-II-120, and SW-II-121 were prepared according to or via methods similar to those described in Examples 1 and 2. The ratios of the lipids were 50:10:38.5:1.5 mol % for the lipid compound of the present invention:phospholipid:cholesterol:PEG-DMG. Tables 1-2 summarize the characteristics and in vivo/in vitro data of the formulations.

The evaluation method of particle size and zeta potential: 50 μL LNP sample was diluted with 950 μL purified water to obtain the diluted LNP sample. The sample was placed in a dynamic light scattering laser particle size analyzer (Malvern, ZS-90) for testing.

Encapsulation efficiency detection method: LNP content and encapsulation efficiency detection were carried out according to the RIBOGREEN kit.

Evaluation method of cytotoxicity: 200 ng of LNP samples were mixed with cells and incubated for 24 h, then the viability was detected using CCK-8 cell viability detection kit. 10 μl of CCK-8 detection reagent was added to the transfected cells and the OD value was detected using a microplate reader at a detection wavelength of 450 nm. The cell viability percentage is obtained by comparing the OD value with that of the cells blank.

In vitro cell expression evaluation method: C2C12 cells, HepG2 cells, A20 cells were cultured, and 100,000 cells per well were plated on a 96-well plate. 100 ng of LNP samples were mixed with the cells and incubated for 24 hours (n=3), and then the expression was detected using luciferase quantitative kit 24 h after administration.

In vivo expression evaluation method: 5 μg Luc (luciferase) mRNA-LNP solution of SW-II-118, SW-120, or SW-121 were injected into the tail vein of Balb/C mice. 6 hours after the administration, the mice are injected 0.15 mg/kg of D-fluorescein substrate intraperitoneally. Within 15 minutes after the injection of the substrate, the fluorescence signal intensity was tested under a small animal in vivo imaging instrument.

TABLE 1
Characteristics of nanoparticle compositions including lipid
compounds of the present invention and different phospholipids

| | Cationic | | | | Zeta potential | Encapsulation |
|---|---|---|---|---|---|---|
| No. | lipid | Phospholipid | Size (nm) | PDI | (mV) | Efficiency (EE, %) |
| 1 | SW-II-118 | DPPE | 265.7 ± 306.1 | 0.54 ± 0.41 | −0.80 ± 0.58 | n. d. |
| 2 | SW-II-118 | DMPE | 102.8 ± 2.3 | 0.29 ± 0.01 | −1.59 ± 0.19 | 86.2 |
| 3 | SW-II-118 | DSPC | 108.7 ± 2.5 | 0.37 ± 0.02 | −1.65 ± 0.58 | 76.0 |
| 4 | SW-II-118 | DAPC | 93.8 ± 1.6 | 0.28 ± 0.03 | −7.18 ± 0.42 | 86.4 |
| 5 | SW-II-118 | DPPC | 97.3 ± 2.6 | 0.26 ± 0.06 | −2.32 ± 0.32 | 83.4 |
| 6 | SW-II-118 | DOPE | 111.3 ± 2.0 | 0.10 ± 0.04 | −4.31 ± 1.36 | 87.5 |
| 7 | SW-II-118 | DSPE | 103.4 ± 2.0 | 0.21 ± 0.01 | −11.97 ± 1.19 | 81.0 |
| 8 | SW-II-120 | DOPE | 142.9 ± 1.9 | 0.13 ± 0.02 | 2.09 ± 0.28 | 90.9 |
| 9 | SW-II-120 | DPPC | 92.97 ± 0.7 | 0.22 ± 0.01 | −3.23 ± 2.95 | 84.9 |
| 10 | SW-II-120 | DAPC | 97.1 ± 1.6 | 0.28 ± 0.02 | −4.82 ± 0.28 | 84.3 |
| 11 | SW-II-120 | DSPC | 88.7 ± 1.1 | 0.20 ± 0.01 | 0.61 ± 0.55 | 85.3 |
| 12 | SW-II-120 | DMPE | 92.7 ± 1.1 | 0.20 ± 0.02 | −6.08 ± 0.43 | 86.2 |
| 13 | SW-II-120 | DSPE | 106.5 ± 5.5 | 0.22 ± 0.02 | −5.15 ± 2.37 | n.d. |
| 14 | SW-II-120 | DPPE | 115.3 ± 3.7 | 0.15 ± 0.04 | −5.04 ± 1.16 | 64.7 |
| 15 | SW-II-121 | DAPC | 96.3 ± 2.2 | 0.27 ± 0.01 | −2.21 ± 0.53 | 76.6 |
| 16 | SW-II-121 | DPPC | 98.7 ± 1.7 | 0.22 ± 0.02 | −1.23 ± 1.43 | 86.7 |
| 17 | SW-II-121 | DMPE | 139.8 ± 1.1 | 0.20 ± 0.03 | 0.01 ± 0.35 | 70.9 |
| 18 | SW-II-121 | DPPE | 98.4 ± 2.7 | 0.17 ± 0.03 | −0.29 ± 0.31 | 84.0 |
| 19 | SW-II-121 | DOPE | 159.87 ± 3.7 | 0.17 ± 0.03 | 5.06 ± 0.15 | 81.5 |
| 20 | SW-II-121 | DSPE | 116.3 ± 0.2 | 0.17 ± 0.03 | −9.60 ± 2.10 | 68.5 |
| 21 | SW-II-121 | DSPC | 98.5 ± 1.5 | 0.22 ± 0.03 | −1.18 ± 1.18 | 86.0 |

n.d. = not determined

It can be seen from Table 1 that the main parameters of SW-II-118, SW-120, and SW-121-LNP are in the range of 80-200 nm (particle size), −10 to +10 mV (potential), 0.1 to 0.3 (PDI), and 60-90% (encapsulation efficiency).

TABLE 2
Cytotoxicity (viability) and Luc expression (in vitro cell expression and in vivo
expression) levels of lipid nanoparticles containing different phospholipids

| | | Phos- | C2C12 cell, | HepG2 cell, in | A20 cell, | | In vivo |
|---|---|---|---|---|---|---|---|
| | Com- | pho- | in vitro | vitro | in vitro | Viability | expression |
| No | pound | lipid | expression | expression | expression | (%) | (p/sec/cm²/sr) |
| 1 | SW-II- | DPPE | 1254.5 ± | 86 ± | 1627.5 ± | 162.7 ± | 25176.7 ± |
| | 118 | | 1458.8 | 72.1 | 2223.8 | 2.2 | 13712.6 |
| 2 | SW-II- | DMPE | 218015.5 ± | 17608.5 ± | 34438 ± | 110.8 ± | 23780.0 ± |
| | 118 | | 1850.5 | 1802.4 | 1566.9 | 3.5 | 7829.9 |
| 3 | SW-II- | DSPC | 80658 ± | 11247.5 ± | 9068 ± | 103.0 ± | 40156666.7 ± |
| | 118 | | 6827.8 | 313.2 | 978.6 | 0.9 | 12959939.6 |
| 4 | SW-II- | DAPC | 32703.5 ± | 6674 ± | 5337 ± | 104.0 ± | 8658333.3 ± |
| | 118 | | 3198.2 | 750.9 | 3787.3 | 1.6 | 10836036.7 |
| 5 | SW-II- | DPPC | 260091 ± | 31083 ± | 12326.5 ± | 105.4 ± | 34323333.3 ± |
| | 118 | | 27654.9 | 8428.7 | 1833.5 | 1.8 | 7208511.2 |
| 6 | SW-II- | DOPE | 300431.5 ± | 17838.5 ± | 2295 ± | 98.7 ± | 38066666.7 ± |
| | 118 | | 39383.7 | 239.7 | 83.4 | 7.5 | 14022515.2 |
| 7 | SW-II- | DSPE | 46290.5 ± | 3053 ± | 3748 ± | 113.9 ± | 25383333.3 ± |
| | 118 | | 478.7 | 574.2 | 841.4 | 3.9 | 2155233.9 |
| 8 | SW-II- | DOPE | 815187.5 ± | 83649.5 ± | 3578 ± | 115.0 ± | 5083666.7 ± |
| | 120 | | 121299.2 | 9702.2 | 45.2 | 7.3 | 870965.2 |
| 9 | SW-II- | DPPC | 147377.5 ± | 24199.5 ± | 7015 ± | 102.9 ± | 21307300.0 ± |
| | 120 | | 43992.6 | 4060.9 | 1564.1 | 4.3 | 18074661.9 |
| 10 | SW-II- | DAPC | 17847.5 ± | 4555.5 ± | 1764 ± | 99.0 ± | 39073333.3 ± |
| | 120 | | 355.7 | 444.8 | 5.6 | 2.9 | 12790349.2 |
| 11 | SW-II- | DSPC | 44819 ± | 13068 ± | 8101.5 ± | 102.3 ± | 58040000.0 ± |
| | 120 | | 17300.1 | 50.9 | 1340.0 | 0.9 | 14955346.9 |
| 12 | SW-II- | DMPE | 228078.5 ± | 31448.5 ± | 37055.5 ± | 87.9 ± | 49736666.7 ± |
| | 120 | | 17648.7 | 6136.9 | 38.9 | 5.0 | 9241928.0 |
| 13 | SW-II- | DSPE | 51132.5 ± | 2482 ± | 1599 ± | 97.9 ± | 128056666.7 ± |
| | 120 | | 38867.5 | 149.9 | 57.9 | 17.4 | 42586895.1 |
| 14 | SW-II- | DPPE | 471290.5 ± | 57343 ± | 54823 ± | 107.1 ± | 7056000.0 ± |
| | 120 | | 5033.9 | 3467.6 | 3462.0 | 9.5 | 1798516 |
| 15 | SW-II- | DAPC | 24183 ± | 3110 ± | 1744.5 ± | 111.1 ± | 156336666.7 ± |
| | 121 | | 6686.4 | 328.1 | 439.1 | 7.1 | 86511427.2 |
| 16 | SW-II- | DPPC | 93988 ± | 16064 ± | 9098 ± | 109.64 ± | 36323333.3 ± |
| | 121 | | 16428.92 | 483.7 | 2282.5 | 5.5 | 28365846.2 |
| 17 | SW-II- | DMPE | 219379.5 ± | 16466 ± | 9110.5 ± | 108.5 ± | 78216666.7 ± |
| | 121 | | 17833.9 | 2066.2 | 1915.6 | 0.5 | 78870447.1 |
| 18 | SW-II- | DPPE | 246921 ± | 25349.5 ± | 27832 ± | 108.0 ± | 46183333.3 ± |
| | 121 | | 35595.7 | 1566.2 | 1871.0 | 0.4 | 34930236.7 |
| 19 | SW-II- | DOPE | 1003834.5 ± | 42994.5 ± | 4244.5 ± | 107.5 ± | 47516666.7 ± |
| | 121 | | 292998.9 | 2537.8 | 1137.7 | 10.0 | 38375638.5 |
| 20 | SW-II- | DSPE | 41556.5 ± | 1211.5 ± | 508.5 ± | 137.0 ± | 19344666.7 ± |
| | 121 | | 12107.8 | 16.3 | 115.2 | 3.4 | 12397347.5 |
| 21 | SW-II- | DSPC | 93333 ± | 7932 ± | 8006.5 ± | 123.3 ± | 12494333.3 ± |
| | 121 | | 25782.5 | 244.6 | 470.2 | 6.3 | 11581077.2 |

It can be seen from Table 2 that the in vitro toxicity results showed that the prepared LNPs had no obvious cytotoxicity, and the relative cell activities were above 80%. The Luc expression activity in C2C12 cells, HepG2 cells, and A20 cells were obvious in vitro.

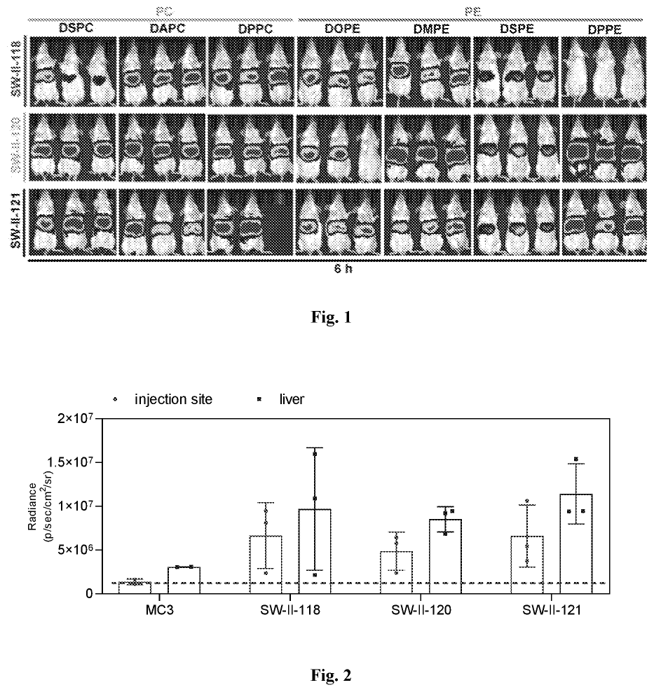

FIG. 1 was a photograph of fluorescence observed under an in vivo imaging instrument upon intravenous administration of SW-II-118, SW-II-120, and SW-II-121-LNP to mice. The phospholipids used in the LNPs were DSPC, DAPC, DPPC, DOPE, DMPE, DSPE, and DPPE, respectively. As can be seen from FIG. 1, all of the LNPs showed significant fluorescence signals, especially when the neutral lipid was DSPC, DAPC, DPPC, DPPE, the fluorescent signal was stronger.

### Example 4: Investigation of Specific Delivery of the Optimized Sample Formulations

The mRNA nanoparticle compositions of SW-II-118, SW-120, and SW-121 were prepared according to the method provided in Example 3. The ratios of the lipids were 50:10:38.5:1.5 mol % for cationic lipid: DSPC: cholesterol: PEG-DMG. The cationic lipid MC3 was a current standard in the art. Accordingly, the standard MC3 formulation including 50 mol % MC3, 10 mol % DSPC, 38.5 mol % cholesterol, and 1.5 mol % PEG-DMG was used as a reference for this study.

5 μg of Luc mRNA-LNP of MC3, SW-II-118, SW-II-120, or SW-II-121 was intramuscularly injected into Balb/C mice (n=3). 6 hours after the administration, the mice were injected intraperitoneally with 0.15 mg/kg D-fluorescein substrate. 15 minutes after the injection of the substrate, the fluorescence signal intensity was tested under a small animal in vivo imaging instrument.

The experimental results were shown in FIG. 2. As can be seen from FIG. 2, the in vivo Luc expressions of Luc mRNA-LNP of SW-II-118, SW-120, or SW-121 was higher than that of MC3 and the Luc expression in liver site was higher than that in injection site.

### Example 5: Expression of Luc Induced by Optimized Sample Formulations

Further bioluminescence studies were performed to investigate the delivery efficiency of nanoparticle compositions including various lipid compounds according to the present invention. The formulations containing reference cationic lipid M62, M63, M118, and M119 (corresponding to Compound 62, 63, 118, and 119 in PCT/US2018/022717 (WO2018/170306), respectively) were evaluated for comparison. Nanoparticle compositions including DSPC as an optimized phospholipid, cholesterol as a structural lipid, PEG-DMG as a PEG lipid, an RNA, and a compound above mentioned were prepared according to or via methods similar to those described in Examples 1 and 2. The ratios of the lipids were 50:10:38.5:1.5 mol % for the lipid of the present invention: DSPC: cholesterol: PEG-DMG.

5 μg of LNP solution containing luciferase mRNA was administered intramuscularly to Balb/C mice (n=3). 6 hours after administration, mice were injected intraperitoneally with 0.15 mg/kg of D-luciferin substrate. Within 15 minutes after the injection of the substrate, the fluorescence signal intensity in injection site and liver site were tested under a small animal in vivo imaging instrument. Tables 3-4 summarize the fluorescence signal intensity of the nanoparticle compositions including present invention lipids and the reference compounds in injection site and liver site.

TABLE 3
Luc expression in injection site of nanoparticle compositions including
compounds according to the present invention in comparison with those
of nanoparticle compositions including reference compounds (wherein
the value is the ratio relative to SW-II-134-3 (set as 1))

| Cationic lipid | I.M. injection site, 6 h |
|---|---|
| M62 | 0.514 |
| M63 | 0.009 |
| M118 | 0.073 |
| M119 | not detectable |
| SW-II-115 | 1.046 |
| SW-II-118 | 1.450 |
| SW-II-120 | 1.064 |
| SW-II-121 | 1.440 |
| SW-II-122 | 1.321 |
| SW-II-127 | 1.743 |
| SW-II-134-1 | 1.899 |
| SW-II-134-2 | 1.734 |
| SW-II-134-3 | 1.000 |
| SW-II-135-1 | 1.771 |
| SW-II-135-2 | 1.028 |
| SW-II-136-2 | 1.110 |
| SW-II-137-3 | 1.358 |
| SW-II-138-1 | 1.972 |
| SW-II-138-2 | 2.826 |
| SW-II-138-3 | 1.294 |
| SW-II-139-1 | 1.807 |
| SW-II-139-2 | 1.440 |
| SW-II-140-1 | 2.450 |
| SW-II-140-2 | 2.257 |

The experimental results were shown in Table 3. The Luc expressions of present invention LNPs in the intramuscular injection site were better than those of reference compounds, showing excellent expression efficiency.

TABLE 4
Luc expression in liver site of nanoparticle compositions including
compounds according to the present invention in comparison with those
of nanoparticle compositions including reference compounds (wherein
the value is the ratio relative to SW-II-137-1 (set as 1))

| Cationic lipid | Liver site upon I.M., 6 h |
|---|---|
| M62 | 0.402 |
| M63 | 0.037 |
| M118 | 0.056 |
| M119 | not detectable |
| SW-II-118 | 2.234 |
| SW-II-120 | 1.318 |
| SW-II-121 | 2.150 |
| SW-II-136-2 | 1.131 |
| SW-II-137-1 | 1.000 |
| SW-II-137-2 | 1.206 |
| SW-II-137-3 | 1.981 |
| SW-II-138-1 | 1.430 |
| SW-II-138-2 | 3.318 |
| SW-II-138-3 | 2.850 |
| SW-II-139-1 | 2.355 |
| SW-II-139-2 | 1.355 |
| SW-II-140-1 | 3.224 |
| SW-II-140-2 | 6.991 |

The experimental results were shown in Table 4. It can be seen from Table 4 that the Luc expression in the liver site upon intramuscular administration of the present invention LNPs were better than that of reference compounds, showing excellent expression efficiency.

### EQUIVALENTS

It is to be understood that while the present invention has been described in conjunction with the detailed description thereof, the foregoing description is intended to illustrate and not limit the scope of the present invention, which is defined by the scope of the appended claims. Other aspects, advantages, and alterations are within the scope of the claims.

1. A lipid compound having the structure as shown in Formula (I): 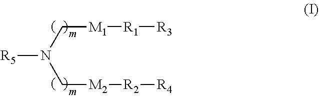
or a pharmaceutically acceptable salt thereof,
wherein,
R₁ and R₂ are each independently selected from the group consisting of C₁-C₁₂ alkyl and C₂-C₁₂ alkenyl;
R₃ and R₄ are each independently selected from the group consisting of C₁-C₁₂ alkyl, C₂-C₁₂ alkenyl, C₆-C₁₀ aryl and 5-10 membered heteroaryl;
provided that at least one of R₃ and R₄ is C₆-C₁₀ aryl or 5-10 membered heteroaryl, and optionally, R₃ and R₄ are each independently substituted by t R₆, where t is an integer selected from 1-5;
R₆ is each independently selected from the group consisting of C₁-C₁₂ alkyl and C₂-C₁₂ alkenyl;
M₁ and M₂ are each independently selected from the group consisting of —OC(O)—, —C(O)O—, —SC(S)—, and —C(S)S—;
R₅ is selected from the group consisting of —C₁₋₁₂ alkylene-Q, Q is selected from the group consisting of —OR₇ and —SR₇, R₇ is independently selected from the group consisting of H, C₁-C₁₂ alkyl, C₂-C₁₂ alkenyl, C₁-C₁₂ alkoxyl, carboxylic acid, sulfinic acid, sulfonic acid, sulfonyl, nitro, cyano, amino, carbamoyl, sulfonamide, C₆-C₁₀ aryl and 5-10 membered heteroaryl;
m and n are each independently an integer selected from 1-12.

2. The lipid compound according to claim 1, wherein,
R₁ and R₂ are each independently selected from the group consisting of C₁-C₁₂ alkyl.

3. The lipid compound according to claim 1 or 2, wherein,
R₃ and R₄ are each independently selected from the group consisting of C₁-C₁₂ alkyl and C₆-C₁₀ aryl;
provided that one of R₃ and R₄ is C₆-C₁₀ aryl, and other is C₁-C₁₂ alkyl;
R₃ and R₄ are each independently substituted by t R₆, where t is an integer of 1-3;
R₆ is each independently selected from the group consisting of C₁-C₁₂ alkyl.

4. The lipid compound according to any one of claims 1-3, wherein,
M₁ and M₂ are each independently selected from the group consisting of —OC(O)— and —C(O)O—.

5. The lipid compound according to any one of claims 1-4, wherein,
R₅ is selected from the group consisting of —C₁₋₅ alkylene-Q, Q is —OH.

6. The lipid compound according to any one of claims 1-5, wherein,
m and n are each independently an integer selected from 2-7.

7. The lipid compound according to any one of claims 1-6, wherein,
R₂ is substituted by R₄ at 1-position or end position; and/or
R₁ is substituted by R₃ at 1-position or end position.

8. The lipid compound according to any one of claims 1-7, wherein,
t is 1 or 2, the benzene ring is substituted by R₆ at meta- and/or para-position, relative to R₁ or R₂.

9. The lipid compound according to any one of claims 1-8, wherein,
t is 1 or 2, R₆ is each independently selected from the group consisting of C₁-C₁₀ alkyl.

10. The lipid compound according to any one of claims 1-9, having the structure as shown in Formula (II): 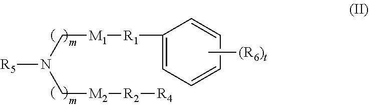
wherein R₁, R₂, R₄, R₅, R₆, M₁, M₂, t, m and n are as defined in any one of claims 1-9;
preferably, in Formula (II)
R₁ is selected from the group consisting of C₁-C₆ alkyl;
R₂ is selected from the group consisting of C₁-C₁₀ alkyl;
R₄ is selected from the group consisting of C₁-C₁₀ alkyl;
M₁ and M₂ are each independently selected from the group consisting of —OC(O)— and —C(O)O—;
R₅ is selected from the group consisting of —C₁₋₅alkylene-Q, Q is selected from the group consisting of —OR₇ and —SR₇, R₇ is independently selected from the group consisting of H, C₁-C₁₂ alkyl and C₂-C₁₂ alkenyl;
m and n are each independently an integer selected from 2-9;
t is an integer selected from 1-3;
R₆ is each independently selected from the group consisting of C₁-C₁₂ alkyl and C₂-C₁₂ alkenyl.

11. The lipid compound according to any one of claims 1-9, having the structure as shown in Formula (III): 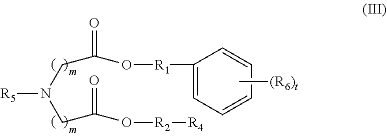
wherein R₁, R₂, R₄, R₅, R₆, t, m and n are as defined in any one of claims 1-9;
preferably, in Formula (III)
R₁ is selected from the group consisting of C₁-C₆ alkyl;
R₂ is selected from the group consisting of C₁-C₁₀ alkyl;
R₄ is selected from the group consisting of C₁-C₁₀ alkyl;
R₅ is selected from the group consisting of —C₁₋₃ alkylene-Q, Q is selected from the group consisting of —OH and —SH;
t is 1 or 2;
R₆ is independently selected from the group consisting of C₁-C₁₂ alkyl and C₂-C₁₂ alkenyl;
m and n are each independently an integer selected from 2-7.

12. The lipid compound according to any one of claims 1-9, having the structure as shown in Formula (IV): 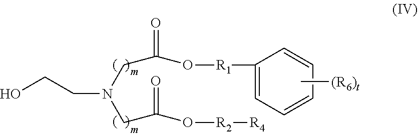
wherein R₁, R₂, R₄, R₆, t, m and n are as defined in any one of claims 1-9;
preferably, in Formula (IV)
R₁ is selected from the group consisting of C₁-C₆ alkyl;
R₂ is selected from the group consisting of C₁-C₁₀ alkyl;
R₄ is selected from the group consisting of C₁-C₁₀ alkyl;
t is 1 or 2;
R₆ is each independently selected from the group consisting of C₁-C₁₂ alkyl and C₂-C₁₂ alkenyl;
m and n are each independently an integer selected from 2-7.

13. The lipid compound according to claim 1, having the following structure:

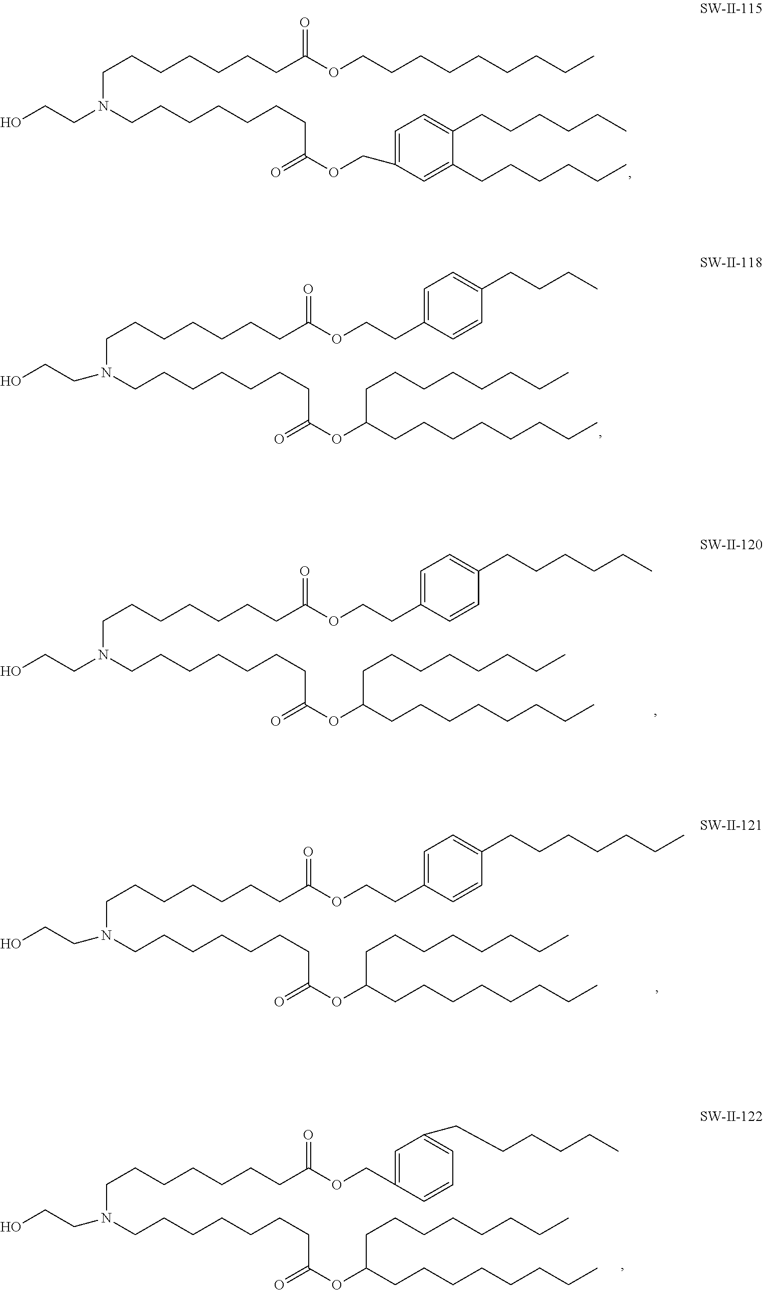 [SMILES: CCCCCCCCCOC(=O)CCCCCCCN(CCO)CCCCCCCC(=O)OCc1ccc(CCCCCC)c(CCCCCC)c1 ; CCCCCCCCC(CCCCCCCC)OC(=O)CCCCCCCN(CCO)CCCCCCCC(=O)OCCc1ccc(CCCC)cc1 ; CCCCCCCCC(CCCCCCCC)OC(=O)CCCCCCCN(CCO)CCCCCCCC(=O)OCCc1ccc(CCCCCC)cc1 ; CCCCCCCCC(CCCCCCCC)OC(=O)CCCCCCCN(CCO)CCCCCCCC(=O)OCCc1ccc(CCCCCCC)cc1 ; CCCCCCCCC(CCCCCCCC)OC(=O)CCCCCCCN(CCO)CCCCCCCC(=O)OCc1cccc(CCCCCC)c1]

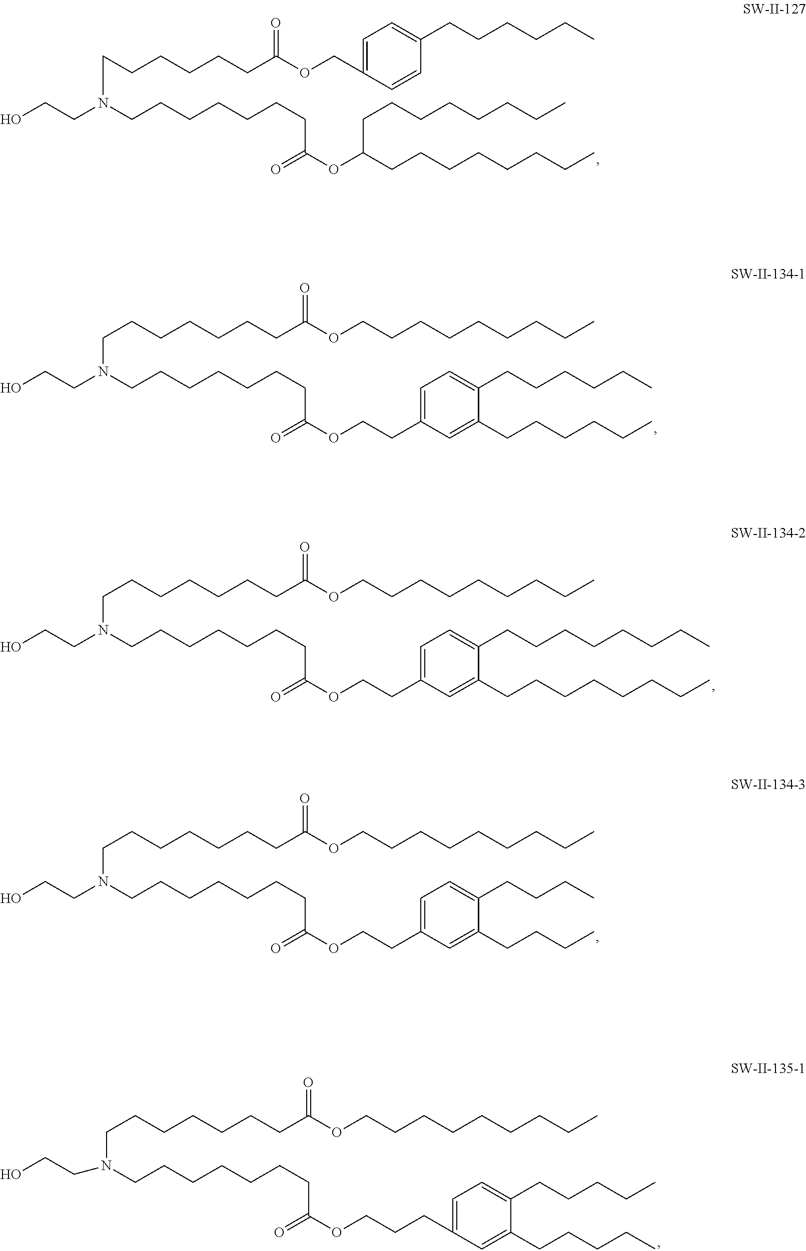 [SMILES: CCCCCCCCC(CCCCCCCC)OC(=O)CCCCCCCN(CCO)CCCCCCC(=O)OCc1ccc(CCCCCC)cc1 ; CCCCCCCCCOC(=O)CCCCCCCN(CCO)CCCCCCCC(=O)OCCc1ccc(CCCCCC)c(CCCCCC)c1 ; CCCCCCCCCOC(=O)CCCCCCCN(CCO)CCCCCCCC(=O)OCCc1ccc(CCCCCCCC)c(CCCCCCCC)c1 ; CCCCCCCCCOC(=O)CCCCCCCN(CCO)CCCCCCCC(=O)OCCc1ccc(CCCC)c(CCCC)c1 ; CCCCCCCCCOC(=O)CCCCCCCN(CCO)CCCCCCCC(=O)OCCCc1ccc(CCCCC)c(CCCCC)c1]

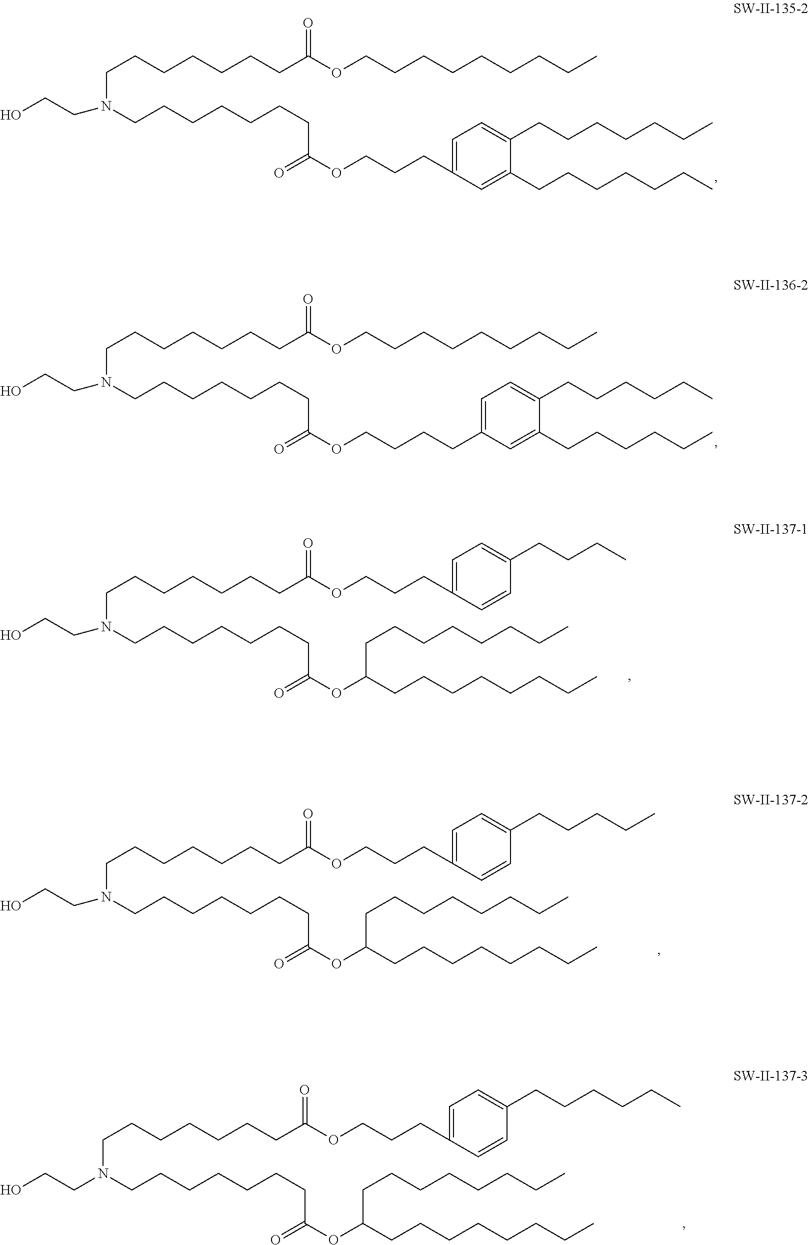 [SMILES: CCCCCCCCCOC(=O)CCCCCCCN(CCO)CCCCCCCC(=O)OCCCc1ccc(CCCCCCC)c(CCCCCCC)c1 ; CCCCCCCCCOC(=O)CCCCCCCN(CCO)CCCCCCCC(=O)OCCCCc1ccc(CCCCCC)c(CCCCCC)c1 ; CCCCCCCCC(CCCCCCCC)OC(=O)CCCCCCCN(CCO)CCCCCCCC(=O)OCCCc1ccc(CCCC)cc1 ; CCCCCCCCC(CCCCCCCC)OC(=O)CCCCCCCN(CCO)CCCCCCCC(=O)OCCCc1ccc(CCCCC)cc1 ; CCCCCCCCC(CCCCCCCC)OC(=O)CCCCCCCN(CCO)CCCCCCCC(=O)OCCCc1ccc(CCCCCC)cc1]

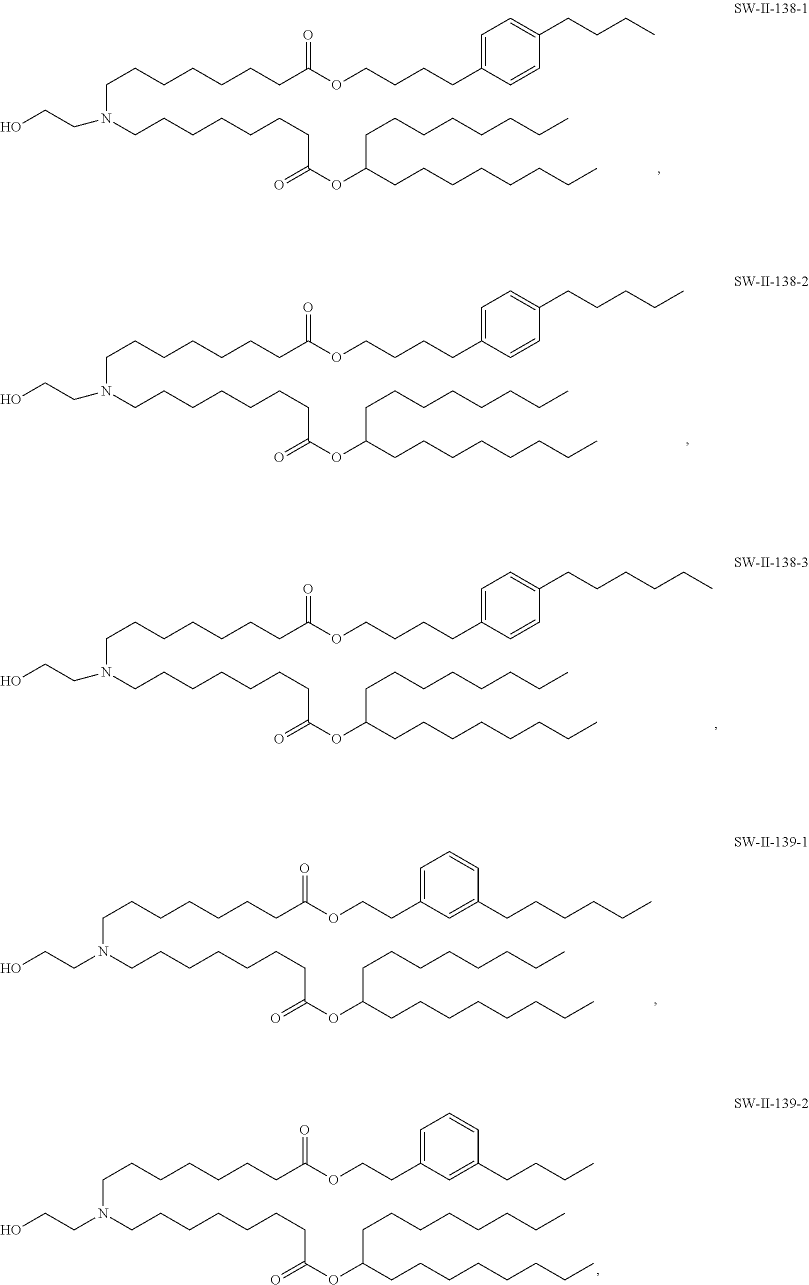 [SMILES: CCCCCCCCC(CCCCCCCC)OC(=O)CCCCCCCN(CCO)CCCCCCCC(=O)OCCCCc1ccc(CCCC)cc1 ; CCCCCCCCC(CCCCCCCC)OC(=O)CCCCCCCN(CCO)CCCCCCCC(=O)OCCCCc1ccc(CCCCC)cc1 ; CCCCCCCCC(CCCCCCCC)OC(=O)CCCCCCCN(CCO)CCCCCCCC(=O)OCCCCc1ccc(CCCCCC)cc1 ; CCCCCCCCC(CCCCCCCC)OC(=O)CCCCCCCN(CCO)CCCCCCCC(=O)OCCc1cccc(CCCCCC)c1 ; CCCCCCCCC(CCCCCCCC)OC(=O)CCCCCCCN(CCO)CCCCCCCC(=O)OCCc1cccc(CCCC)c1]

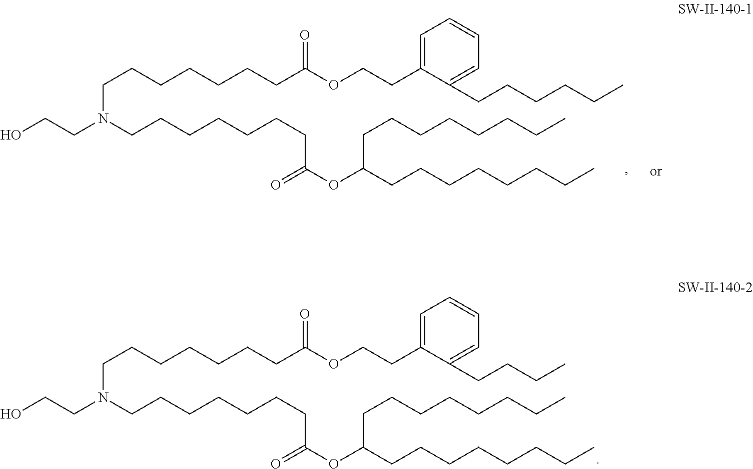 [SMILES: CCCCCCCCC(CCCCCCCC)OC(=O)CCCCCCCN(CCO)CCCCCCCC(=O)OCCc1ccccc1CCCCCC ; CCCCCCCCC(CCCCCCCC)OC(=O)CCCCCCCN(CCO)CCCCCCCC(=O)OCCc1ccccc1CCCC]

14. A nanoparticle composition, comprising the lipid compound according to any one of claims 1-13 or a pharmaceutically acceptable salt thereof, and a therapeutic or prophylactic agent.

15. The nanoparticle composition according to claim 14, wherein the therapeutic or prophylactic agent is a nucleic acid, for example RNA, particularly mRNA.

16. The nanoparticle composition according to claim 14 or 15, having a core-shell structure, wherein the therapeutic or prophylactic agent is a nucleic acid, which is included in a polymeric complex or protein core, and the polymeric complex or protein core itself is encapsulated in a biocompatible lipid bilayer shell.

17. The nanoparticle composition according to claim 16, wherein the polymeric complex or protein core particle comprises a positively charged polymer or protein, preferably, the positively charged polymer or protein comprises protamine, polyethyleneimine, poly(β-aminoester) or a combination thereof.

18. The nanoparticle composition according to any one of claims 14-17, comprising DSPC, DAPC, DPPC, DOPE, DMPE, DSPE, DPPE or any combination thereof, particularly DSPC, DAPC, DPPC, DPPE or any combination thereof.

19. A pharmaceutical composition, comprising the lipid compound according to any one of claims 1-13 or a pharmaceutically acceptable salt thereof, or the nanoparticle composition according to any one of claims 14-18, and an optional pharmaceutically acceptable excipient.

20. A method of delivering a therapeutic or prophylactic agent to a cell of a mammalian subject, the method comprising administering to the subject the composition according to any one of claims 14-19, said administering comprising contacting the cell with the composition, whereby the therapeutic and/or prophylactic agent is delivered to the cell.

21. A method of producing a polypeptide of interest in a cell in a mammalian subject, the method comprising contacting the cell with the composition according to any one of claims 14-19, wherein the therapeutic or prophylactic agent is a mRNA, and wherein the mRNA encodes the polypeptide of interest, whereby the mRNA is capable of being translated in the cell to produce the polypeptide of interest.

22. Use of the nanoparticle composition according to any one of claims 14-18 or the pharmaceutical composition according to claim 19 for the manufacture of a medicament for treating or preventing a disease or disorder in a subject in need thereof.

23. The use according to claim 22, wherein the disease or disorder is characterized by dysfunctional or aberrant protein or polypeptide activity.

24. The use according to claim 23, wherein the disease or disorder is selected from the group consisting of rare diseases, infectious diseases, cancer and proliferative diseases, genetic diseases, autoimmune diseases, diabetes, neurodegenerative diseases, cardio- and reno-vascular diseases, and metabolic diseases.

25. The use or method according to any one of claims 20-24, wherein the subject is a mammalian, particularly a human.
